SEQUENCE LISTING (138 sequences)

SEQ ID NO: 1 - protein, 6 aa, chimeric HIV-HRV
ELDKWA

SEQ ID NO: 2 - protein, 6 aa, chimeric HIV-HRV
ALDKWA

SEQ ID NO: 3 - protein, 5 aa, chimeric HIV-HRV
LDKWA

SEQ ID NO: 4 - protein, 9 aa, chimeric HIV-HRV
LELDKWASL

SEQ ID NO: 5 - protein, 13 aa, chimeric HIV-HRV
RCFALDKWATSCS

SEQ ID NO: 6 - protein, 10 aa, chimeric HIV-HRV
SCLALDKWAL

SEQ ID NO: 7 - protein, 13 aa, chimeric HIV-HRV
  misc_feature (10)..(10): Xaa can be any naturally occurring amino acid
PCAELDKWAXTXN

SEQ ID NO: 8 - protein, 13 aa, chimeric HIV-HRV
  misc_feature (11)..(11): Xaa can be any naturally occurring amino acid
CCSALDKWAIXCK

SEQ ID NO: 9 - protein, 13 aa, chimeric HIV-HRV
  misc_feature (1)..(1): Xaa can be any naturally occurring amino acid
XCVELDKWASXCX

SEQ ID NO: 10 - protein, 15 aa, chimeric HIV-HRV
  misc_feature (1)..(1): Xaa can be any naturally occurring amino acid
XCGELDKWAXSCXXG

SEQ ID NO: 11 - protein, 22 aa, chimeric HIV-HRV
EKNEQDLLALDKWANLWNWFNI

SEQ ID NO: 12 - protein, 14 aa, chimeric HIV-HRV
YSALDKWALGSSCD

SEQ ID NO: 13 - protein, 12 aa, chimeric HIV-HRV
PCGALDKWASCP

SEQ ID NO: 14 - protein, 12 aa, chimeric HIV-HRV
PCSELDKWAACP

SEQ ID NO: 15 - protein, 12 aa, chimeric HIV-HRV
GCSALDKWANCP

SEQ ID NO: 16 - protein, 13 aa, chimeric HIV-HRV
GCSALDKWAKLCQ

SEQ ID NO: 17 - protein, 13 aa, chimeric HIV-HRV
PCVELDKWASTCS

SEQ ID NO: 18 - protein, 13 aa, chimeric HIV-HRV
PCLELDKWASMCD

SEQ ID NO: 19 - protein, 13 aa, chimeric HIV-HRV
GCGELDKWARVCD

SEQ ID NO: 20 - protein, 15 aa, chimeric HIV-HRV
DCSELDKWARECECV

SEQ ID NO: 21 - protein, 15 aa, chimeric HIV-HRV
PCSALDKWAAQRKCE

SEQ ID NO: 22 - protein, 15 aa, chimeric HIV-HRV
GCLALDKWAPQCICD

SEQ ID NO: 23 - protein, 15 aa, chimeric HIV-HRV
PCSALDKWAQRQACS

SEQ ID NO: 24 - protein, 15 aa, chimeric HIV-HRV
PCSALDKWASPPTCI

SEQ ID NO: 25 - protein, 15 aa, chimeric HIV-HRV
PCVELDKWAELTNCP

SEQ ID NO: 26 - protein, 15 aa, chimeric HIV-HRV
PCLELDKWASSDNCP

SEQ ID NO: 27 - protein, 15 aa, chimeric HIV-HRV
PCSALDKWATEVTCR

SEQ ID NO: 28 - protein, 14 aa, chimeric HIV-HRV
DCGALDKWAPASCE

SEQ ID NO: 29 - protein, 15 aa, chimeric HIV-HRV
PCCALDKWATLNNCN

SEQ ID NO: 30 - protein, 15 aa, chimeric HIV-HRV
GCGELDKWASIKNCI

SEQ ID NO: 31 - protein, 15 aa, chimeric HIV-HRV
PCIELDKWAAAGNCP

SEQ ID NO: 32 - protein, 15 aa, chimeric HIV-HRV
PCKELDKWAQLGTCP

SEQ ID NO: 33 - protein, 15 aa, chimeric HIV-HRV
PCSALDKWACGNKCS

SEQ ID NO: 34 - protein, 15 aa, chimeric HIV-HRV
PCGELDKWASPANCH

SEQ ID NO: 35 - protein, 15 aa, chimeric HIV-HRV
PCVALDKWACLTSCL

SEQ ID NO: 36 - protein, 15 aa, chimeric HIV-HRV
TCSALDKWAQVENCQ

SEQ ID NO: 37 - protein, 15 aa, chimeric HIV-HRV
GCSALDKWAQPENCP

SEQ ID NO: 38 - protein, 16 aa, chimeric HIV-HRV
PCSELDKWWAPALKCL

SEQ ID NO: 39 - protein, 15 aa, chimeric HIV-HRV
PCAALDKWASSSTCK

SEQ ID NO: 40 - protein, 15 aa, chimeric HIV-HRV
PCLELDKGASLRKCD

SEQ ID NO: 41 - protein, 15 aa, chimeric HIV-HRV
PCLELDKWAASSTCT

SEQ ID NO: 42 - protein, 15 aa, chimeric HIV-HRV
PCGELDKWATSSNCV

SEQ ID NO: 43 - protein, 15 aa, chimeric HIV-HRV
PCLALDKWASQRDCD

SEQ ID NO: 44 - protein, 15 aa, chimeric HIV-HRV
PCSELDKWATVSNCP

SEQ ID NO: 45 - protein, 15 aa, chimeric HIV-HRV
PCSELDKWAKSRECL

SEQ ID NO: 46 - protein, 15 aa, chimeric HIV-HRV
PCSALDKWAPLGNCE

SEQ ID NO: 47 - protein, 15 aa, chimeric HIV-HRV
PCSELDKWAALSKCP

SEQ ID NO: 48 - protein, 14 aa, chimeric HIV-HRV
PCPALDKWASSHCQ

SEQ ID NO: 49 - protein, 15 aa, chimeric HIV-HRV
PCGALDKWASSPDCS

SEQ ID NO: 50 - protein, 15 aa, chimeric HIV-HRV
PCFELDKWANRSQCE

SEQ ID NO: 51 - protein, 17 aa, chimeric HIV-HRV
PCCALDKWAHVSTSLCN

SEQ ID NO: 52 - protein, 14 aa, chimeric HIV-HRV
PCGELDKWATTHCS

SEQ ID NO: 53 - protein, 15 aa, chimeric HIV-HRV
SCCELDKWAKPGACQ

SEQ ID NO: 54 - protein, 17 aa, chimeric HIV-HRV
GCPALDKWASVSHGCCD

SEQ ID NO: 55 - protein, 16 aa, chimeric HIV-HRV
PCAALDKWATLAPACP

SEQ ID NO: 56 - protein, 15 aa, chimeric HIV-HRV
GCSALDKWATVDTCP

SEQ ID NO: 57 - protein, 13 aa, chimeric HIV-HRV
PCSLELDKWAPCT

SEQ ID NO: 58 - protein, 13 aa, chimeric HIV-HRV
PCSLELDKWAACT

SEQ ID NO: 59 - protein, 13 aa, chimeric HIV-HRV
PCPAALDKWASCT

SEQ ID NO: 60 - protein, 13 aa, chimeric HIV-HRV
PCLSELDKWAECA

SEQ ID NO: 61 - protein, 14 aa, chimeric HIV-HRV
PCSMELDKWASSCT

SEQ ID NO: 62 - protein, 14 aa, chimeric HIV-HRV
PCGLELDKWAKACQ

SEQ ID NO: 63 - protein, 14 aa, chimeric HIV-HRV
PCQLELDKWATQCQ

SEQ ID NO: 64 - protein, 14 aa, chimeric HIV-HRV
PCLPELDKWAVQCP

SEQ ID NO: 65 - protein, 14 aa, chimeric HIV-HRV
PCSLALDKWAHDCE

SEQ ID NO: 66 - protein, 14 aa, chimeric HIV-HRV
PCSTELDKWANKCD

SEQ ID NO: 67 - protein, 16 aa, chimeric HIV-HRV
PCLSALDKWASKGKCT

SEQ ID NO: 68 - protein, 16 aa, chimeric HIV-HRV
GCSTELDKWATSSKCL

SEQ ID NO: 69 - protein, 16 aa, chimeric HIV-HRV
PCYSALDKWANVSNCN

SEQ ID NO: 70 - protein, 18 aa, chimeric HIV-HRV
PCLLELDKWASQATHSCP

SEQ ID NO: 71 - protein, 18 aa, chimeric HIV-HRV
PCFTELDKWASSPPPLCP

SEQ ID NO: 72 - protein, 18 aa, chimeric HIV-HRV
PCVGALDKWASAEKANCS

SEQ ID NO: 73 - protein, 15 aa, chimeric HIV-HRV
  misc_feature (5)..(5): Xaa can be any naturally occurring amino acid
GCPGXSGLDKWAACN

SEQ ID NO: 74 - protein, 15 aa, chimeric HIV-HRV
GCCDVSGLDKWASCQ

SEQ ID NO: 75 - protein, 15 aa, chimeric HIV-HRV
PCCDSSVLDKWATCT

SEQ ID NO: 76 - protein, 16 aa, chimeric HIV-HRV
PCHELMVLDKWAPQCQ

SEQ ID NO: 77 - protein, 18 aa, chimeric HIV-HRV
SCDEFCVLDKWAETCKCV

SEQ ID NO: 78 - protein, 18 aa, chimeric HIV-HRV
GCTESPVLDKWADMCTCT

SEQ ID NO: 79 - protein, 18 aa, chimeric HIV-HRV
PCPGNNELDKWAALGTCD

SEQ ID NO: 80 - protein, 18 aa, chimeric HIV-HRV
GCNHLTELDKWAKSETCD

SEQ ID NO: 81 - protein, 18 aa, chimeric HIV-HRV
DCPEINALDKWAAMRACS

SEQ ID NO: 82 - protein, 18 aa, chimeric HIV-HRV
GCPDVAGLDKWAPLSTCQ

SEQ ID NO: 83 - protein, 18 aa, chimeric HIV-HRV
GCQESGELDKWAKSRTCL

SEQ ID NO: 84 - protein, 18 aa, chimeric HIV-HRV
PCHESSVLDKWAAKASCT

SEQ ID NO: 85 - protein, 17 aa, chimeric HIV-HRV
GCDHSVELDWAKLSTCP

SEQ ID NO: 86 - protein, 18 aa, chimeric HIV-HRV
PCGESVELDKWASISRCP

SEQ ID NO: 87 - protein, 17 aa, chimeric HIV-HRV
ECGDLVLDKWASVDQCV

SEQ ID NO: 88 - protein, 18 aa, chimeric HIV-HRV
PCPGNNELDKWAALGTCD

SEQ ID NO: 89 - protein, 20 aa, chimeric HIV-HRV
TCQHVGGLDKWASQRSLSCP

SEQ ID NO: 90 - protein, 20 aa, chimeric HIV-HRV
PCFDSSALDKWASPSTTSCP

SEQ ID NO: 91 - protein, 17 aa, chimeric HIV-HRV
SCHGHGTSELDKWACCD

SEQ ID NO: 92 - protein, 17 aa, chimeric HIV-HRV
PCSEYGLAELDKWAPCE

SEQ ID NO: 93 - protein, 17 aa, chimeric HIV-HRV
DCNEGSKGELDKWASCA

SEQ ID NO: 94 - protein, 17 aa, chimeric HIV-HRV
SCDNVGDLALDKWATCP

SEQ ID NO: 95 - protein, 18 aa, chimeric HIV-HRV
PCVVDNSLALDKWASTCE

SEQ ID NO: 96 - protein, 18 aa, chimeric HIV-HRV
PCSDSHVLALDKWADFCA

SEQ ID NO: 97 - protein, 18 aa, chimeric HIV-HRV
LCDYEDLTALDKWAASCA

SEQ ID NO: 98 - protein, 18 aa, chimeric HIV-HRV
PCVYDNSMELDKWAIQCN

SEQ ID NO: 99 - protein, 19 aa, chimeric HIV-HRV
VCCELESLELDKWAAQPCI

SEQ ID NO: 100 - protein, 20 aa, chimeric HIV-HRV
PCNVQELCALDKWAPSSSCP

SEQ ID NO: 101 - protein, 20 aa, chimeric HIV-HRV
PCNEPEVPALDKWASPPLCP

SEQ ID NO: 102 - protein, 20 aa, chimeric HIV-HRV
VCNEQERLELDKWASLRACN

SEQ ID NO: 103 - protein, 20 aa, chimeric HIV-HRV
  misc_feature (17)..(17): Xaa can be any naturally occurring amino acid
SCNEKEYVELDKWASFXNCA

SEQ ID NO: 104 - protein, 20 aa, chimeric HIV-HRV
PCGDLDGIALDKWASISDCS

SEQ ID NO: 105 - protein, 20 aa, chimeric HIV-HRV
SCDEQGSVALDKWAALSNCP

SEQ ID NO: 106 - protein, 20 aa, chimeric HIV-HRV
KCDDQEVFELDKWATLTPCP

SEQ ID NO: 107 - protein, 20 aa, chimeric HIV-HRV
KCDDQEVFELDKWAALRPCP

SEQ ID NO: 108 - protein, 20 aa, chimeric HIV-HRV
DCNRSDVLELDKWANSPNCQ

SEQ ID NO: 109 - protein, 20 aa, chimeric HIV-HRV
MCNVQHSSALDKWAHSQLCE

SEQ ID NO: 110 - protein, 22 aa, chimeric HIV-HRV
TCVEDNVSELDKWASVPASLCS

SEQ ID NO: 111 - protein, 22 aa, chimeric HIV-HRV
SCNEQHLSALDKWATPRNPPCQ

SEQ ID NO: 112 - protein, 12 aa, chimeric HIV-HRV
  misc_feature (12)..(12): Xaa can be any naturally occurring amino acid
CVELDKWASTCX

SEQ ID NO: 113 - protein, 17 aa, chimeric HIV-HRV
SCDEFCVLDKWAETCKC

SEQ ID NO: 114 - protein, 14 aa, chimeric HIV-HRV
EQELLELDKWASLW

SEQ ID NO: 115 - protein, 12 aa, chimeric HIV-HRV
SVHLGPGRAFYA

SEQ ID NO: 116 - protein, 16 aa, chimeric HIV-HRV
CENEQELLELDKWASL

SEQ ID NO: 117 - protein, 15 aa, chimeric HIV-HRV
CNEQELLEKDKWADL

SEQ ID NO: 118 - protein, 22 aa, HIV-1
EKNEQELLELDKWASLWNWFNI

SEQ ID NO: 119 - DNA, 51 bp, HIV Oligomer N1
  misc_feature: m is a or c
ggtgaacgtggtatcgatttatcannktgtnndgmactggacaagtgggca

SEQ ID NO: 120 - DNA, 54 bp, HIV oligomer N2
  misc_feature: m is a or c
ggtgaacgtggtatcgatttatcannktgtnndnndgmactggacaagtgggca

SEQ ID NO: 121 - DNA, 60 bp, HIV Oligomer N4
  misc_feature: m is a or c
ggtgaacgtggtatcgatttatcannktgtnndnndnndnndgmactggacaagtgggca

SEQ ID NO: 122 - DNA, 66 bp, HIV Oligomer N6
  misc_feature: m is a or c
ggtgaacgtggtatcgatttatcannktgtnndnndnndnndnndnndgmactggacaagtgggca

SEQ ID NO: 123 - DNA, 51 bp, HIV Oligomer C1
  misc_feature: m is a or c
aacatctttaacgggccctccaacmnnacahnntgcccacttgtccagtkc

SEQ ID NO: 124 - DNA, 54 bp, HIV Oligomer C2
  misc_feature: m is a or c
aacatctttaacgggccctccaacmnnacahnnhnntgcccacttgtccagtkc

SEQ ID NO: 125 - DNA, 60 bp, HIV Oligomer C4
  misc_feature: m is a or c
aacatctttaacgggccctccaacmnnacahnnmnnhnnhnntgcccacttgtccagtkc

SEQ ID NO: 126 - DNA, 66 bp, HIV Oligomer C6
  misc_feature: m is a or c
aacatctttaacgggccctccaacmnnacamnnmnnhnnmnnhnnhnntgcccacttgtccagtkc

SEQ ID NO: 127 - DNA, 28 bp, HRV NIm-II Region Oligomer SPE II (N-Terminal)
cactcactagtaagatcaggttacacag

SEQ ID NO: 128 - DNA, 28 bp, HRV NIm-II Region Oligomer OPC (C-Terminal)
gtatcactattgtggctgtattattggt

SEQ ID NO: 129 - protein, 18 aa, HIV-1 (clade B consensus)
NEQELLELDKWASLWNWF

SEQ ID NO: 130 - protein, 21 aa, HIV-1 MN
EKNEQELLELDKWASLWNWFD

SEQ ID NO: 131 - protein, 22 aa, HIV-1 NL43
EKNEQELLELDKWASLWNWFNI

SEQ ID NO: 132 - protein, 22 aa, HIV-1 JRCSF
EKNEQELLELDKWASLWNWFGI

SEQ ID NO: 133 - protein, 21 aa, HIV-1QZ4589
EKNEQELLELDKWASLWSWFD

SEQ ID NO: 134 - protein, 21 aa, HIV-192HT594
EKNEQDLLELDKWASLWNWFN

SEQ ID NO: 135 - protein, 21 aa, HIV-1 92RW009
EKNEQDLLALDKWANLWNWFD

SEQ ID NO: 136 - protein, 21 aa, HIV-1 92UG037
EKNEQELLELDKWANLWNWFD

SEQ ID NO: 137 - protein, 22 aa, HIV-1 92UG005
EKNEQELLQLDKWASLWNWFSD

SEQ ID NO: 138 - protein, 21 aa, HIV-1 92TH024
DRNEKDLLELDKWASLWSWFD

(19) United States
(12) Patent Application Publication
(10) Pub. No.: US 2006/0088549 A1
(43) Pub. Date: Apr. 27, 2006

(54) Chimeric virus vaccine

(76) Inventors: Gail F. Arnold, Belle Mead, NJ (US); Eddy Arnold, Belle Mead, NJ (US)

Correspondence Address: STEVEN B. STEIN, 114 OLD BLOOMFIELD AVENUE, PARSIPPANY, NJ, 07054, US

(21) Appl. No.: 11/176,182

(22) Filed: Jul. 7, 2005

Related U.S. Application Data
(60) Provisional application No. 60/585,823, filed on Jul. 8, 2004.

(51) Int. Cl. A61K 39/12; C12Q 1/70; C07H 21/04; C12N 7/00; C07K 14/095

(52) U.S. Cl. 424/199.1; 435/5; 435/69.3; 435/456; 435/235.1; 435/325; 530/350; 530/388.3; 536/23.72

(57) ABSTRACT

The present invention provides an immunogenic composition comprising: (a) an isolated recombinant chimeric human rhinovirus, wherein the recombinant chimeric human rhinovirus comprises (i) a nucleic acid having a nucleotide sequence of a human rhinovirus encoding at least a portion of a human rhinovirus capsid; (ii) a heterologous nucleic acid having a nucleotide sequence encoding a chimeric region, wherein the chimeric region is expressed on the surface of the chimeric rhinovirus and is capable of participating in an immune reaction; and (iii)a pharmaceutically acceptable carrier.

This application claims priority to U.S. Provisional Application, Ser. No. 60/585,823, filed Jul. 8, 2004, which is incorporated by reference in its entirety. 10 Throughout this application, various publications are referenced by name or by number.

This invention was made with the support of the National Institutes of Health/NAID Grant Nos. NIH-NIAID R01 AI 38221 (7/95-1/01) and NIH-NIAID R2 AI 45353 (6/99-5/01). The United States Government may have certain rights to this invention.

Full citations for these publications may be found listed at the end of the specification and preceding the claims. The disclosures of these publications in their entireties are hereby incorporated by reference into this application in order to more fully describe the state of the art. A Sequence Listing is also provided.

### FIELD OF THE INVENTION

The present invention relates to recombinant chimeric human virus vaccines which can be used to stimulate an immune response to the chimeric virus and thus serve to provide protection against infection by the virus elements, which comprise the chimera vaccine. In particular, the present invention provides a immunogenic composition comprising a chimeric human rhinovirus expressing a portion of HIV-1 gp41, capable of stimulating an immune response to the chimeric virus and specifically against HIV.

### BACKGROUND OF THE INVENTION

The production of effective and safe vaccines against harmful viruses and other pathogens continues to be a difficult endeavor. To date, the most successful vaccines involve the use of inactivated viruses or live attenuated viruses obtained from multiple passages of wild-type viruses in tissue culture or in non-human primates. A concern that still lingers, however, is that outbreaks occasionally occur, apparently from improper inactivation of the viruses, reversion or pseudoreversion of the viruses to virulent strains, extension of the host range, and/or contamination of vaccines with live virus.

To overcome some of these complications, considerable research effort has been expended to examine the feasibility and efficacy of immunizing with empty viral capsids and pathogen-derived proteins and peptides. Unfortunately, the antigenicity profiles for complex virions and empty capsids are often quite different. This phenomenon has been documented for several picornaviruses, including rhinovirus (Lonberg-Holm & Yin, J. Virol., 12:114-123, 1973), poliovirus (Mayer, et al., J. Immunol., 78:435-455, 1957), foot-and-mouth disease virus (FMDV) (Rowlands, et al., J. Gen. Virol., 26:227-238, 1975), and Coxsackie B virus (Frommhagen, J. Immunol., 95:818-822, 1965). Studies with individual virion proteins have shortcomings as well. Individual coat proteins, for instance, have antigenic determinants absent from intact viruses (Wiegers & Derrick, J. Gen. Virol., 64:777-785, 1983) and are generally far less effective at stimulating neutralizing antibodies than are whole virions. Attempts to use peptides to provide protection against dangerous pathogens have also been disappointing. Despite occasional examples of success (e.g., Bittle, et al., Nature, 298:30-33, 1982; Pfaff, et al., EMBO. J., 1:869-874, 1982), most peptides fail to protect vaccinated animals, even when they are capable of stimulating the production of neutralizing antibodies (e.g., Ada & Skehel, Nature, 316:764-765, 1985; Tiollais, et al., Nature, 317:489-495, 1985; DiMarchi, et al., Science, 232:639-641, 1986).

A more recent approach to vaccine development and the one, which is utilized in the present invention, uses chimeric viruses or virus-like particles (VLPs) as vehicles for presentation of foreign antigens to the immune system. A number of virus-like particles (VLPs) composed of fusion proteins have been shown to test positively in standard enzyme-linked immunosorbent assays (ELISAs) with antibodies directed against either substituent of the fusion protein. Among chimeric VLPs, few have been tested for their ability to protect infected animals; an exceptional case involved the testing of a hepatitis B surface antigen:poliovirus VPI chimera which, when injected into mice, produced only weak protection against poliovirus (Delpeyroux, et al., Science, 233:472-475, 1986). Live recombinant viruses with the composite antigenicity of mixed poliovirus types have been produced in other laboratories (Kohara, et al., J. Virol., 62:2828-2835, 1988; Martin, et al., EMBO. J., 7:2839-2847, 1988; Burke, et al., J. Gen. Virol., 70:2475-2479, 1989); these reports gave no reference to testing of these live chimeras for protection against the virus. In another experiment, Evans, et al., (Nature, 339:385-388, (1989)) incorporated an epitope (positions 735-752) from the transmembrane glycoprotein of HIV-1, gp41, into the neutralizing antigenic region of VPI (NAg-1) of poliovirus 1 Sabin. These workers reported that rabbit antisera and monoclonal antibodies elicited by the chimera were capable of neutralizing in vitro a wide range of American and African isolates of HIV-1. Protection studies were not reported with this construct.

Unfortunately, previously developed chimeric viruses, such as those based on poliovirus and vaccinia virus, have certain characteristics, which make them less than ideal. One of the most significant drawbacks to these chimeric viruses is that their effectiveness as vaccines is limited since many individuals already have a significant immune response to the native virus. Also, at least in the case of poliovirus, the use of a live vaccine carries with it the fact that the native virus is a major pathogen with associated risks.

A cDNA clone of HRV14 was reported by Mizutani and Colonno (J. Virol., 56:628, 1985). This clone of HRV14 purportedly has been utilized to make single-site mutations in the coat proteins primarily for the purpose of examining the properties of the cell receptor attachment site on the viral surface (Colonno, et al. 1988). The construction of human rhinovirus chimeras has been reported, however, no such construct has been identified as providing the desired protective effect. (See U.S. Pat. Nos. 5,541,100 and 5,714,374). Indeed, the largely unpredictable nature of chimeric virion assembly necessitates a method for generating and screening large numbers of chimeric constructs capable of eliciting the desired immunogenic effects.

An improved way to stimulate an immune response using a chimeric virus would be to utilize a native virus which, (1) is a relatively mild pathogen, such that little risk would be associated with the use of a live chimeric virus vaccine; (2) has a broad range of serotypic diversity (>100 serotypes), lessening the likelihood of preexisting immunity and thereby enabling vaccination in adults; and (3) has the ability to stimulate a significant neutralizing immune response in mucosal membranes as well as in serum.

The human immunodeficiency virus (HIV-1) has been established as the primary etiologic agent in the pathogenesis of acquired immunodeficiency syndrome (AIDS) and related disorders. (Barre-Sinoussi, et al. Science (1983) 220:868-871; Gallo, et al., Science (1984) 224:500-503; Levy, et al., Science (1984) 225:840-842). HIV is a frequently mutating retrovirus that attacks the human immune system. Infection of humans with HIV-1 leads to a humoral immune response by B lymphocytes resulting in the production of antibodies directed against most of the viral structural antigens. A particular subset of antibodies is directed against HIV envelope antigens (gp120 and gp41) which may be involved in induction of active immunity. (Matthews, et al., AIDS Research and Human Retroviruses (1987) 3:197-206).

It has proven exceedingly difficult to develop an effective vaccine against HIV; conventional approaches have proven ineffective in stimulating a broad acting, protective immunity. The present invention provides a means for accomplishing this result.

Specifically, the present invention provides a chimeric virus vaccine comprising a cold-causing human rhinovirus type 14 (HRV14) used to display the the HIV gp41 ELDKWA epitope, in ways to elicit neutralizing antibodies against diverse isolates of HIV-1. Immunogenic compositions comprising HRV14:HIV-1 chimeras are provided which elicit protection against HIV.

### SUMMARY OF THE INVENTION

The present invention provides an immunogenic composition comprising: (a) an isolated recombinant chimeric human rhinovirus, wherein the recombinant chimeric human rhinovirus comprises (i) a nucleic acid having a nucleotide sequence of a human rhinovirus encoding at least a portion of a human rhinovirus capsid; (ii) a heterologous nucleic acid having a nucleotide sequence encoding a chimeric region, wherein the chimeric region is expressed on the surface of the chimeric rhinovirus and is capable of participating in an immune reaction; and (iii)a pharmaceutically acceptable carrier.

The present invention also provides a plasmid capable of generating an infectious recombinant rhinovirus cDNA having the following characteristics: (a) it encodes a polypeptide capable of forming a rhinovirus capsid or portion thereof; and (b) it has the ability to hybridize under standard hybridization conditions to a nucleic acid sequence or complement thereof, capable of encoding the sequence shown in SEQ ID NO.: 1.

Further, the present invention provides a kit for producing the provided recombinant rhinovirus comprising (a) the provided infectious cDNA; and (b) a coupled transcription and translation system.

Still further the present invention provides a method for generating the provided immunogenic composition comprising the steps of (a) generating nucleic acid mixture comprising: (i) a nucleotide sequence of a human rhinovirus encoding at least a portion of a human rhinovirus capsid; (ii) a nucleotide flanking sequence 3′ to a chimeric sequence insertion site; (iii) a nucleotide flanking sequence site 5′ to the chimeric sequence insertion site, wherein the nucleotide sequences flanking the chimeric site comprise a pseudo random selection of nucleotides, capable of encoding a selection of amino acids; (iv)a heterologous nucleic acid chimeric insertion sequence comprising a nucleotide sequence, inserted into the chimeric site, wherein the heterologous nucleic acid chimeric insertion sequence comprises a pseudo random selection of nucleotides, capable of encoding a selection of amino acids; (b) Isolating viable chimeric virus expressing the chimeric region; and (c) Selecting virus, wherein the chimeric rhinovirus is capable of participating in an immune reaction.

### BRIEF DESCRIPTION OF THE DRAWINGS

FIG. 1. Amino acid sequences corresponding to the HIV-1-gp4 I ELDKWA (SEQ. ID. NO.:1) region and the six N— and C-terminal residues proximal to it in 622 isolates from the Los Alamos Database. Variations in the ELDKWA sequence are not shown reflecting intent to conserve the sequence in the present invention.

FIG. 2. Sequences of the oligonucleotides used for the synthesis of 16 combinatorial HRV14:HIV-1 gp41 ELDKWA libraries. The N— and C-terminal oligonucleotides were designed to hybridize in the region encoding ELDKWA (shaded sequence). The randomized residues are designated by the codon NN(G_{0.5}/T_{0.5}) or its complement (C_{0.5}/A_{0.5})NN. Biased residues (50% randomized and 50% HIV-1 sequences) are encoded in the nucleotide proportions representative of those of the desired residues at the corresponding positions in HIV-1.

FIG. 3. Oligonucleotides used for sequencing of HRV14:HIV-1 gp41 ELDKWA chimeric plasmids and for the generation and sequencing of chimeric viral reverse transcriptase (RT) products.

FIGS. 4A-B. Scheme used to immunize guinea pigs with HRV14:HIV-1 gp41 ELDKWA chimeras. All animals received at least three immunizations. In selected cases, animals received a combination of a virus and peptide boost at week 9, and/or a peptide boost at week 13. In these cases, a third blood sample was collected at week 16 to evaluate the reactivity of the immune sera to the boosting peptide.

FIGS. 5A-C. Chimeric HRV14:HIV-1 ELDKWA libraries. FIG. 5A. Design of 16 chimeric HRV 14:HIV-1 ELDKWA libraries showing set of eight overlapping oligonucleotides (represented as amino acid translations) coding for the core ELDKWA epitope and N— or C-terminal linkers of differing lengths and sequences. Hybridization and extension of the 16 possible pairings produced 16 double-stranded DNA library inserts. X=any of the 20 amino acids; B=biased=50% X, 50% HIV-1 sequence(s) found at the corresponding site (in proportion to the amino acid representation in the Los Alamos Database (Korber, et al, 1999)). FIG. 5B. Amino acid composition and proportions represented for each biased residue. FIG. 5C. Sequences in the ELDKWA region of 100 unselected and immunoselected clones from the HRV14:HIV-1 gp41 ELDKWA libraries.

FIG. 6. Sequence analysis of the ELDKWA inserts contained in 100 chimeric HRV14:gp41 ELDKWA virus clones.

FIG. 7. Immunoselection scheme used to enrich for chimeric viruses with optimal presentations of ELDKWA.

FIG. 8. Flowchart illustrating the scheme for producing and characterizing chimeric rhinovirus libraries.

FIG. 9. Competitive immunoselection for chimeric viruses that efficiently bind mAb 2F5. Chimeras that effectively display the ELDKWA epitope are able to bind mAb2F5 in the presence of high concentrations of competing LELDKWASL (SEQ. ID. NO.:4) peptide.

FIGS. 10A-B. Binding of Pool I and the chimeric virus pools derived from its immunoselection to mAb 2F5. ELISA titers were obtained using 0.1 μg of immobilized 2F5. FIG. 10A. Binding of immunoselected pools from rounds A and B. FIG. 10B. Binding of the most antigenic immunoselected pool from round B (B128), and pools obtained from immunoselection round C. Pools A, B and C were immunoselected with 0.1, 0.05, and 0.025 μg/ml 2F5, respectively. The numbers next to the letter designate the pmol/well of competing peptide used for immunoselection of that particular pool (1 pmol/well=0.01 μM total peptide concentration see FIG. 7).

FIG. 11. Cell elution vs. peptide elution of immunoselected chimeric viruses. The cell elution process relies on the ability of the antibody-bound virus to infect H1-HeLa cells, likely preventing some of the most tightly bound viruses being able to productively infect the cells and be harvested. The peptide elution relies on the addition of high concentrations of LELDKWASL (SEQ. ID. NO.:4) peptide, with the aim of freeing the tightly bound viruses from antibody.

FIGS. 12A-B. Binding of Pool II and the chimeric virus pools derived from its immunoselection to human mAb 2F5. ELISA titers were obtained using 0.1 μg of immobilized 2F5. FIG. 12A. Binding of immunoselected pools from rounds A and B; FIG. 12B. Binding of immunoselected pools from round B and BN. Pools A and B were immunoselected with 0.025 and 0.0125 μg/ml of 2F5 respectively. The numbers next to the letter designate the pmol/well of competing peptide used to immunoselect that particular pool (1 pmol/well=0.01 μM total peptide concentration; see FIG. 7). BN pools were immunoselected with 0.0125 μg/ml of 2F5 with no corresponding peptide during selection, and eluted with LELDKWASL peptide, with the numbers of pmol of eluting peptide next to the BN indication (see FIG. 7).

FIG. 13. Binding of Pool III and the chimeric virus pools derived from its immunoselection to human mAb 2F5. ELISA titers were obtained using 0.1 μg of immobilized 2F5. Binding of immunoselected pools from rounds A, B and AN. Pools A and B were immunoselected with 0.05 and 0.025 μg/ml of 2F5 respectively. The numbers next to the letter designate the pmol/well of competing peptide used to immunoselect that particular pool (1 pmol/well=0.01 μM total peptide concentration; see FIG. 7). AN pools were immunoselected with 0.05 μg/ml of 2F5 with no corresponding peptide during selection, and eluted with LELDKWASL peptide, with the numbers of pmol of eluting peptide next to the AN indication (see FIG. 7).

FIGS. 14A-B. Binding and neutralization of individual HRV14:HIV-1 gp41 ELDKWA chimeras immunoselected from Pool I by mAb 2F5. FIG. 14A. ELISA titers were obtained using 0.1 μg of immobilized 2F5. The undiluted virus stock contained 1×10⁸ PFU/ml. FIG. 14B. Neutralization of immunoselected viruses and parent pool (Pool I) by mAb 2F5.

FIGS. 15A-B. Binding and neutralization of individual HRV14:HIV-1 gp41 ELDKWA chimeras immunoselected from Pool II by mAb 2F5. FIG. 15A. ELISA titers were obtained using 0.1 μg of immobilized 2F5. The undiluted virus stock contained 1×10⁸ PFU/ml. FIG. 15B. Neutralization of immunoselected viruses and parent pool (Pool II) by mAb 2F5.

FIGS. 16A-B. Binding and neutralization of individual HRV14:HIV-1 gp41 ELDKWA chimeras immunoselected from Pool III by mAb 2F5. FIG. 16A. ELISA titers were obtained using 0.1 μg of immobilized 2F5. The undiluted virus stock contained 1×10⁸ PFU/ml. FIG. 16B. Neutralization of immunoselected viruses and their parent pool (Pool III) by mAb 2F5.

FIG. 17. Binding of anti-chimera guinea pig serum samples to immobilized biotinylated LELDKWASL peptide. Samples taken 7 weeks post immunization. Samples were also taken 12 weeks post immunization (not shown). Samples are designated by the name of the chimeric virus used for guinea pig immunization followed by the serum code in parenthesis. Samples with the same color correspond to animals immunized with the same chimeric virus. 14-C4000-1-P corresponds to sera elicited by an animal boosted with the designated chimera and the 14-mer peptide at week 9. α-HRV14 corresponds to sera elicited by wild-type HRV14. α-AH3.67 corresponds to sera elicited by a previously made ELDKWA chimera that contained an insert with sequence NELDKWAS. The prebleed consists of an equal mixture of the sera derived from the 12 immunized guinea pigs prior to inoculation with chimeric virus.

FIG. 18. Binding of guinea pig anti-chimera IgG to the LELDKWASL peptide in the presence of competing Linear I peptide. A non-saturating amount of anti-chimera IgG was bound to immobilized biotinylated LELDKWASL peptide in the presence of sequential dilutions of a competing peptide with the sequence CENEQELLELDKWASL. Samples are designated by the name of the chimeric virus used for guinea pig immunization followed by the serum code in parenthesis.

FIG. 19. Binding of guinea pig anti-chimera IgG to the LELDKWASL peptide in the presence of competing Lac- I peptide. A non-saturating amount of anti-chimera IgG was bound to immobilized biotinylated LELDKWASL peptide in the presence of sequential dilutions of a competing cyclic peptide with the sequence CNEQELLEKDKWADL-NH₂ (with a lactam bridge connecting the underlined K and D residues). Samples are designated by the name of the chimeric virus used for guinea pig immunization followed by the serum code in parenthesis.

FIG. 20. Comparison of 2F5-binding titers of chimeric viruses, neutralizing titers of immunoselected chimeras by 2F5, and LELDKWASL-binding activity of anti-chimera IgG fractions.

FIG. 21. Antigenicity and immunogenicity assays performed on immunoselected HRV14:HIV-1 gp41 ELDKWA chimeras

FIGS. 22A-B. HRV14:HIV-1 gp41 ELDKWA virus-derived serum neutralization titers against diverse HIV-1 isolates.

### DETAILED DESCRIPTION OF THE INVENTION

The present invention provides an immunogenic composition comprising: (a) an isolated recombinant chimeric human rhinovirus, wherein the recombinant chimeric human rhinovirus comprises (i) a nucleic acid having a nucleotide sequence of a human rhinovirus encoding at least a portion of a human rhinovirus capsid; (ii) a heterologous nucleic acid having a nucleotide sequence encoding a chimeric region, wherein the chimeric region is expressed on the surface of the chimeric rhinovirus and is capable of participating in an immune reaction; and (iii)a pharmaceutically acceptable carrier.

According to an embodiment of this invention, the nucleotide sequence encoding the rhinovirus capsid encodes at least part of a rhinovirus neutralizing immunogenic site. According to still another embodiment of this invention, the recombinant chimeric rhinovirus is viable. According to an alternative embodiment of this invention, the recombinant chimeric rhinovirus is non-viable. It is specifically contemplated and understood by one of skill in the art that a viable recombinant chimeric rhinovirus may be inactivated by means well understood in the art, while retaining its immunogenic properties. According to still another embodiment of this invention, the recombinant chimeric rhinovirus is biologically pure. Isolation of a biologically pure virus stock is well understood in the art and the present invention specifically contemplates that a biologically pure virus stock may be produced by any number of methods.

According to a preferred embodiment of the present invention, the recombinant chimeric rhinovirus is comprising a nucleic acid or portion thereof having the following characteristics: (a) it encodes a polypeptide capable of forming a human rhinovirus capsid or portion thereof; and (b) it has the ability to hybridize under standard hybridization conditions to a nucleic acid sequence or complement thereof, capable of encoding the sequence shown in SEQ ID NO.: 1.

According to another preferred embodiment of the present invention, the recombinant chimeric rhinovirus is comprising a nucleic acid or portion thereof having the following characteristics: (a) it encodes a polypeptide capable of forming a human rhinovirus capsid or portion thereof; and (b) it has the ability to hybridize under standard hybridization conditions to a nucleic acid sequence or complement thereof, capable of encoding the sequence shown in SEQ ID NO.: 2. It is specifically contemplated by the present invention that other sequences selected from the gp41 region of HIV-1 may be selected as the chimeric sequence.

The present invention also provides a plasmid capable of generating an infectious recombinant rhinovirus cDNA having the following characteristics: (a) it encodes a polypeptide capable of forming a rhinovirus capsid or portion thereof; and (b) it has the ability to hybridize under standard hybridization conditions to a nucleic acid sequence or complement thereof, capable of encoding the sequence shown in SEQ ID NO.: 1. According to an alternative embodiment, the present invention provides a plasmid capable of generating an infectious recombinant rhinovirus cDNA having the following characteristics: (a) it encodes a polypeptide capable of forming a rhinovirus capsid or portion thereof; and (b) it has the ability to hybridize under standard hybridization conditions to a nucleic acid sequence or complement thereof, capable of encoding the sequence shown in SEQ ID NO.: 2. It is specifically contemplated by the present invention that other sequences selected from the gp41 region of HIV-1 may be selected as the chimeric sequence.

According to yet another embodiment, the present invention provides an isolated, infectious cDNA of the provided recombinant rhinovirus. Infectious cDNA are well described in the art as are methods for infectious cDNA generation and method of transfection. According to still yet another embodiment, the present invention provides the provided infectious cDNA incorporated within a vector. According to even another embodiment of this invention, the vector is a plasmid. It is specifically contemplated that the vector may include appropriate expression and replication control elements. Another embodiment provides a prokaryotic host cell transformed with the provided vector. Suitable vectors are well known and described in the art. An alternative embodiment provides a eukaryotic host cell transformed with the provided vector. Still another embodiment provides an isolated infectious RNA transcribed from the provided vector. It is specifically contemplated that the RNA may be isolated from a cell or a cell-free system. It is also specifically contemplated that nucleic acid corresponding to the recombinant virus sequence may be permanently introduced into a cell line capable of producing recombinant virus or proteins thereof, by any number of methods well described in the art.

Accordingly, the present invention provides an isolated cell line comprising the provided recombinant virus nucleotide sequence. According to a preferred embodiment, the nucleotide sequence is derived from the provided cDNA. A still further embodiment of this invention provides an isolated recombinant human rhinovirus produced from the provided RNA. Still yet another embodiment provides an isolated recombinant human rhinovirus produced from the provided cell line. A preferred embodiment of the present invention provides a vaccine comprising a unit dose of an immunogenic composition comprising the provided recombinant virus. Even further still, the present invention provides a method of inducing an immune response comprising the step of administering the unit dose of the provided immunogenic composition to a vaccinee.

According to another embodiment of the present invention, the recombinant rhinovirus is constructed by inserting into the nucleotide sequence of a human rhinovirus encoding part of a neutralizing immunogenic site, a heterologous nucleotide sequence encoding a chimeric region, wherein the chimeric region is expressed on the surface of the chimeric rhinovirus and is capable of participating in an immune reaction. According to an embodiment of this invention, the chimeric region is presented in the NIm-II portion of viral protein VP2. According to another embodiment of this invention, the chimeric region is presented in the NIm-IA portion of viral protein VP1. It will be understood that according to a preferred embodiment of this invention, the chimeric region is of viral origin. According to a preferred embodiment of this invention, the viral origin of the chimeric region is a retrovirus. A preferred embodiment of this invention is wherein the retrovirus is a human immunodeficiency virus. A most preferred embodiment is wherein the human immunodeficiency virus is selected from the group consisting of HIV-1 and HIV-2. According to a preferred embodiment of this invention, the chimeric region of the human immunodeficiency virus is selected from the group consisting of the gag and env proteins.

According to an embodiment of this invention, at least a portion of the chimeric region as translated comprises at least a portion of the gp120 envelope glycoprotein of HIV-1. According to an embodiment of this invention, at least a portion of the chimeric region as translated comprises at least a portion of the V3 loop of the gp120 envelope glycoprotein of HIV-1. According to a preferred embodiment of this invention, at least a portion of the chimeric region as translated comprises at least a portion of the gp41 envelope glycoprotein of HIV-1. According to a preferred embodiment of this invention, at least a portion of the chimeric region as translated comprises at least a portion of the sequence of SEQ. ID. NO.: 1 of the gp41 envelope glycoprotein of HIV-1. According to a preferred embodiment of this invention, at least a portion of the chimeric region as translated comprises at least a portion of the sequence of SEQ. ID. NO.:2 of the gp41 envelope glycoprotein of HIV-1. According to another embodiment of this invention, the chimeric region as translated comprises at least a portion of the sequence selected from the sequential group consisting of SEQ. ID. NO.:3 through SEQ. ID. NO.: 118, inclusive. This is understood by one of skill in the art to include each sequence listed from SEQ. ID. No.:3 through SEQ. ID. No.:118. According to an embodiment of the present invention, the chimeric region is presented at loop 2 of the NIm-II immunogenic site of HRV14 viral protein VP2. According to another embodiment of this invention, the chimeric region is presented between from about amino acid 159 to about amino acid 161 of VP2. The present invention also provides the chimeric rhinovirus wherein at least a portion of the chimeric region as translated comprises (SEQ ID NO: 1). The present invention also provides the chimeric rhinovirus wherein at least a portion of the chimeric region as translated comprises (SEQ ID NO:2).

Still further, the present invention provides a kit for producing the provided recombinant rhinovirus comprising (a) the provided infectious cDNA; and (b) a coupled transcription and translation system.

According to an embodiment of this invention, the kit is further comprising a cellular expression system. According to still another embodiment of this invention, the coupled transcription and translation system further comprises: (a) the provided nucleic acid; (b) a eukaryotic cell free cell extract, wherein the extract is from either an animal or a plant cell; (c) ribonucleotide triphosphates; and (d) RNA polymerase.

Still further the present invention provides a method for generating the provided immunogenic composition comprising the steps of (a) generating nucleic acid mixture comprising: (i) a nucleotide sequence of a human rhinovirus encoding at least a portion of a human rhinovirus capsid; (ii) a nucleotide flanking sequence 3′ to a chimeric sequence insertion site; (iii) a nucleotide flanking sequence site 5′ to the chimeric sequence insertion site, wherein the nucleotide sequences flanking the chimeric site comprise a pseudo random selection of nucleotides, capable of encoding a selection of amino acids; (iv)a heterologous nucleic acid chimeric insertion sequence comprising a nucleotide sequence, inserted into the chimeric site, wherein the heterologous nucleic acid chimeric insertion sequence comprises a pseudo random selection of nucleotides, capable of encoding a selection of amino acids; (b) Isolating viable chimeric virus expressing the chimeric region; and (c) Selecting virus, wherein the chimeric rhinovirus is capable of participating in an immune reaction.

The present invention also provides a method of inducing an immune response comprising the step of administering a unit dose of the provided immunogenic composition to a vaccinee. One embodiment of this invention, is further comprising a step of boosting immunization with at least one peptide encoded by a nucleic acid having the ability to hybridize under standard hybridization conditions to a nucleic acid sequence or complement thereof, capable of encoding the sequence shown in SEQ ID NO.: 1. According to a preferred embodiment of this invention, the peptide is KLH-conjugated. Another embodiment of this invention is further comprising a step of boosting immunization with at least one peptide encoded by a nucleic acid having the ability to hybridize under standard hybridization conditions to a nucleic acid sequence or complement thereof, capable of encoding the sequence shown in SEQ ID NO.: 2. According to a preferred embodiment of this invention, the peptide is KLH-conjugated.

Yet further still, the present invention provides an isolated monoclonal antibody, which recognizes the provided chimeric rhinovirus. According to an emobidment of this invention, the epitope that binds or is recognized by the monoclonal antibody is within at least a portion of SEQ is NO: 1. According to an alternative embodiment of this invention, the epitope that binds or is recognized by the monoclonal antibody is within at least a portion of SEQ is NO: 2. According to a preferred embodiment, the monoclonal antibody binds HIV. It is specifically understood that the HIV may be virus isolated from an in vitro culture, from a cell-free system or from a human subject. The HIV may be isolated and biologically pure or may be a heterogenous mixture of samples. According to still another embodiment, the invention provides an antibody, which competes with the provided monoclonal antibody for binding to HIV. It is understood by one of skill in the art how to identify such an antibody by any of the well-described competition assays. Finally, the present invention provides a monoclonal antibody producing cell line that produces the provided monoclonal antibody.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994)]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1 986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The amino acid residues described herein are preferred to be in the “L” isomeric form. However, residues in the “D” isomeric form can be substituted for any L-amino acid residue, as long as the desired fuctional property of immunoglobulin binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, abbreviations for amino acid residues are used as shown in shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations, which may appear alternately herein.

It should also be noted that in addition to the standard IUPAC one-letter code for the nucleotides of DNA the following code is used herein including letters for ambiguity as follows: M is A or C; R is A or G; W is A or T; S is C or G; Y is C or T; K is G or T; V is A, C or G; H is A, C or T; D is A, G or T; B is C, G or T; and N is G, A, T or C.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly with regard to potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large-scale animal culture.

Transcriptional and translational control sequences are DNA regulatory sequences, such as promoters, enhancers, polyadenylation signals, terminators, and the like, that provide for the expression of a coding sequence in a host cell. A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease SI), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used generally herein, such as in referring to probes prepared and used in the present invention, is defined as a molecule comprised of two or more ribonucleotides, preferably more than three. Its exact size will depend upon many factors, which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

“Degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or Cuu or CUC |
| | or CUA or CUG |
| Isoleucine (Ile or I) | AUU or AUC or AUA |
| Methionine (Met or M) | AUG |
| Valine (Val or V) | GUU or GUC or GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in the nucleotide sequence encoding SEQ. ID. NO: 1 or SEQ. ID. NO:2 or other sequences described herein, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

Alanine
Valine
Leucine
Isoleucine
Proline
Phenylalanine
Tryptophan
Methionine
   Amino Acids with Uncharged Polar R Groups
Glycine
Serine
Threonine
Cysteine
Tyrosine
Asparagine
Glutamine
   Amino Acids with Charged Polar R Groups (Negatively Charged at ph 6.0)
Aspartic acid
Glutamic acid
   Basic Amino Acids (Positively Charged at pH 6.0)
Lysine
Arginine
Histidine (at pH 6.0) 10
   Another Grouping may be Those Amino Acids with Phenyl Groups:
Phenylalanine
Tryptophan
Tyrosine

Another Grouping may be According to Molecular Weight (i.e., Size of R Groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tm with washes of higher stringency, if desired.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890. Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an elastin-binding portion thereof.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose and 20 mm glutamine.

It is contemplated that the proteins, peptides, nucleic acids, vectors and virus particles of this invention can be administered to a subject to impart a therapeutic or beneficial effect. Therefore, the proteins, peptides, nucleic acids, vectors and particles of this invention can be present in a pharmaceutically acceptable carrier.

“Pharmaceutically acceptable” means that a material that is not biologically or otherwise undesirable, i.e., the material may be administered to a subject, along with the nucleic acid or vector of this invention, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art (see, e.g., Remington's Pharmaceutical Science; latest edition).

Pharmaceutical formulations of this invention, such as vaccines, of the present invention can comprise an immunogenic amount of the virus particles as disclosed herein in combination with a pharmaceutically acceptable carrier. An “immunogenic amount” is an amount of the virus particles sufficient to evoke an immune response (humoral and/or cellular immune response) in the subject to which the pharmaceutical formulation is administered.

Exemplary pharmaceutically acceptable carriers include, but are not limited to, sterile pyrogen-free water and sterile pyrogen-free physiological saline solution.

Subjects which may be administered immunogenic amounts of the virus particles of the present invention include, but are not limited to, human and animal (e.g., horse, donkey, mouse, hamster, monkey) subjects.

Administration may be by any suitable means, such as intraperitoneal or intramuscular injection.

Pharmaceutical formulations for the present invention can include those suitable for parenteral (e.g., subcutaneous, intradermal, intramuscular, intravenous and intraarticular) administration. Alternatively, pharmaceutical formulations of the present invention may be suitable for administration to the mucous membranes of a subject (e.g., intranasal administration). The formulations may be conveniently prepared in unit dosage form and may be prepared by any of the methods well known in the art.

Thus, the present invention provides a method for delivering nucleic acids and vectors (e.g., virus particles) encoding the antigens of this invention to a cell, comprising administering the nucleic acids or vectors to a cell under conditions whereby the nucleic acids are expressed, thereby delivering the antigens of this invention to the cell. The nucleic acids can be delivered as naked DNA or in a vector (which can be a viral vector) or other delivery vehicles and can be delivered to cells in vivo and/or ex vivo by a variety of mechanisms well known in the art (e.g., uptake of naked DNA, viral infection, liposome fusion, endocytosis and the like). The cell can be any cell which can take up and express exogenous nucleic acids.

As used herein, “pM” means picomolar, “nM” means nanmolar, “uM” means micromolar, “mM” means millimolar, “ul” means microliter, “ml” means milliliter, “l” means liter.

As used herein, the term “synthetic amino acid” means an amino acid which is chemically synthesized and is not one of the 20 amino acids naturally occurring in nature. As used herein, the terms “non-natural amino acid” and “unnatural amino acid” means an amino acid, which is not one of the 20 amino acids naturally occurring in nature. Thus, a synthetic amino acid is an unnatural amino acid.

As used herein, the term “biosynthetic amino acid” means an amino acid found in nature other than the 20 amino acids commonly described and understood in the art as “natural amino acids.” Examples of “non-amide isosteres” include but are not limited to secondary amine, ketone, carbon-carbon, thioether, and ether moieties.

As used herein, the term “non-natural peptide analog” means a variant peptide comprising a synthetic amino acid. As used herein, “NMR” means nuclear magnetic resonance, “ESMS” means electrospray mass spectrometry; “CBD” means chitin binding, domain; “SH2” means src homology type-2 domain; “Abl” means human Abelson protein tyrosine kinase, “GST” means glutathione S-transferase; “HSQC” means heteronuclear single-quantum correlation spectroscopy. “HPLX” means high pressure liquid chromatography; “PhSH” means thiophenol, “BzISH” means benzyl mercaptan; standard single and triple letter codes for amino acids, and single letter codes for nucleic acids are used throughout.

A “segment” as the term is used herein, consists of a portion of a protein or peptide primary amino acid sequence. Such a segment as used herein may be generated by proteolytic cleavage, chemical cleavage or physical disruption. Alternatively, such a segment may be generated by an expression vector or by an in vitro translation of an RNA transcript or portion thereof. Such a segment may assume a structural conformation or folding pattern which is unique to the segment or which represents the conformation of the segment in the complete protein or peptide.

A “domain” as used herein, is a portion of a protein that has a tertiary structure. The domain may be connected to other domains in the complete protein by short flexible regions of polypeptide. Alternatively, the domain may represent a functional portion of the protein.

As used herein, amino acid residues are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. Abbreviations for amino acid residues are used in keeping with standard polypeptide nomenclature delineated in J. Biol. Chem., 243:3552-59 (1969).

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

Amino acids with nonpolar R groups include: Alanine, Valine, Leucine, Isoleucine, Proline, Phenylalanine, Tryptophan and Methionine. Amino acids with uncharged polar R groups include: Glycine, Serine, Threonine, Cysteine, Tyrosine, Asparagine and Glutamine. Amino acids with charged polar R groups (negatively charged at pH 6.0) include: Aspartic acid and Glutamic acid. Basic amino acids (positively charged at pH 6.0) include: Lysine, Arginine and Histidine (at pH 6.0). Amino acids with phenyl groups include: Phenylalanine, Tryptophan and Tyrosine. Particularly preferred substitutions are: Lys for Arg and vice versa such that a positive charge may be maintained; Glu for Asp and vice versa such that a negative charge may be maintained; Ser for Thr such that a free —OH can be maintained; and Gin for Asn such that a free NH₂ can be maintained. Amino acids can be in the “D” or “L” configuration. Use of peptidomimetics may involve the incorporation of a non-amino acid residue with non-amide linkages at a given position.

Amino acid substitutions may also be introduced to substitute an amino acid with a particularly preferable property. For example, a Cys may be introduced as a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

The detectable marker labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The proteins and peptides of the present invention can also be labeled with a radioactive element or with an enzyme. The radioactive label can be detected by any of the currently available counting procedures. The preferred isotope may be selected from ³H, ¹³C, ¹⁵N, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I , and ¹⁸⁶Re.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A basic description of nucleic acid amplification or PCR (polymerase chain reaction) is described in Mullis, U.S. Pat. No. 4,683,202, which is incorporated herein by reference. The amplification reaction uses a template nucleic acid contained in a sample, two primer sequences and inducing agents. The extension product of one primer when hybridized to the second primer becomes a template for the production of a complementary extension product and vice versa, and the process is repeated as often as is necessary to produce a detectable amount of the sequence.

The inducing agent may be any compound or system which will function to accomplish the synthesis of primer extension products, including enzymes. Suitable enzymes for this purpose include, for example, E. coli DNA polymerase I, thermostable Taq DNA polymerase, Kienow fragment of E. coli DNA polymerase I, T4 DNA polymerase, other available DNA polymerases, reverse transcriptase and other enzymes which will facilitate combination of the nucleotides in the proper manner to form amplification products. The oligonucleotide primers can be synthesized by automated instruments sold by a variety of manufacturers or can be commercially prepared based upon the nucleic acid sequence of this invention.

As used herein, the term “chip” means any solid support including, but not limited to silicon, glass, polypropylene, polystyrene, cellulose, plastic and paper. Accordingly, the term “protein chip” means a protein covalently bound to a solid support including, but not limited to silicon, glass, polypropylene, polystyrene, cellulose, plastic and paper. The “protein” component of a protein chip as used herein is the ligation product of an oligopeptide and a recombinantly expressed protein or portion thereof, the peptide being the component covalently bound to the solid support. Additionally, as used herein, the term “antibody chip” means an antibody or the antigen-binding portion thereof covalently bound to a solid support as the ligation product of an oligopeptide and a recombinantly expressed antibody protein or portion thereof, the peptide being the component covalently bound to the solid support. Furthermore, as used herein, the term “antigen chip” means an antigen covalently bound to a solid support as the ligation product of an oligopeptide and a recombinantly expressed antigenic protein or portion thereof, the peptide being the component covalently bound to the solid support. Moreover, the term “protein chip protein” refers to the protein component of the protein chip which is the ligation product produced by the methods disclosed by the present invention.

Reagents

Plasmids. Two plasmids used were p3XBIA and p3IIST. The plasmid p3XBIA contains a cDNA of the complete HRV 14 genome, a poly(A) tail, and the T7 promoter. This plasmid was used to generate wild-type HRV14 for an experimental control. The p3IIST plasmid was derived from the plasmid p3IAIIHA, which was a p3XBIA-derived plasmid engineered to contain the restriction sites Apa I and Cla I flanking the region encoding the largest of three surface loops of the NIm-II site (as well as sequences encoding an influenza hemagglutinin (HA) epitope inserted between the restriction sites; Arnold et al., 1994). The p3IIST plasmid, instead of encoding the HA epitope, contains a series of translation stop codons in all three reading frames between the Apa I and Cla I restriction sites (Smith et al., 1994). The presence of the “stop” cassette is a precaution to avoid the production of wild-type infectious RNAs from non-recombinant plasmids. This plasmid was used for the insertion of HIV-1 gp41 ELDKWA sequences within the region encoding for the NIm-II site.

Bacteria. The bacterial strain used for electroporation and amplification of the engineered plasmids was E. coli DH10B ElectroMax (Gibco BRL/Invitrogen, Cat. No. 18290-015; Grant et al., 1990).

Viruses and Pseudoviruses. HRV14 provided by Dr. Roland Rueckert (Institute of Molecular Virology, University of Wisconsin) and Dr. Michael Rossmann (Purdue University). Recombinant pseudoviruses expressing HIV-1 envelope proteins were engineered and produced in Christos J. Petropoulos' research laboratory (Virologic, San Diego; Richman et al., 2003).

Human and Mammalian Cells. H1-HeLa cells (Conant and Hamparian, 1968) were used for the production, propagation, titering, and microtiter neutralization assays of both wild-type HRV14 and recombinant HRV14 HIV-1 gp41-ELDKWA chimeric viruses. They were kindly provided by Dr. Roland Rueckert. Cells used for producing the recombinant pseudoviruses encoding HIV-1 env were HEK 293 (Pear, 1993). U87 astroglioma (Ponten & Macintyre, 1968) cells were used for the pseudovirus neutralization assays to characterize polyclonal antibody responses from guinea pigs inoculated with HRV14 HIV-1 gp41 ELDKWA chimeras.

Media and Buffers. Luria Bertani (LB) (Miller, 1972) medium is I% Bacto Tryptone (Difco), 5% Bacto Yeast (Difco), and 1% NaCl and was used to grow plasmids. LB supplemented with 100 μg/ml of Ampicillin (Sigma-Aldrich Cat No. A9393) is designated as LB-Amp. LB-Agar is LB with (1.5% (w/v) of Bacto Agar (Difco). SOB medium is 20% Bacto tryptone (Difco), 5% Bacto yeast extract (Difco), 8.5 mM NaCl, and 2.5 mM KCl, pH 7.0. SOC medium used to grow E. coli after electroporation is SOB medium supplemented with 10 mM MgCl₂, and 20 mM glucose immediately before use.

Dulbecco's Modified Eagle Medium (D-MEM), used to wash and keep H1-HeLa cells after viral transfection, is from Gibco BRL/Invitrogen (Cat. No. 1965-092). Medium M used for propagating virus in H1-HeLa cells is MEM (Gibco BRL/Invitrogen cat. No. 11090-081) supplemented with 10 mM N-2-hydroxyethylpiperazine-N′-2-ethane sulfonic acid (HEPES), pH 7.3, 0.1 M nonessential amino acids (NEAA), 4 mM L-glutamine (L-Gln), and 100 unit/ml penicillin-streptomycin. PA medium used for viral plaque assays is MEM (Gibco BRL/Invitrogen cat. No. 11090-081) supplemented with 20 mM HEPES, pH 7.3, 10 mM MgCl₂, 100 units/ml penicillin-streptomycin, 4 mM L-Gln, and 1-20% heat-inactivated fetal bovine serum (FBS). RPMI-H9 medium is RPMI 1640 (Gibco BRL/Invitrogen Cat No. 21870-076) supplemented with 4 mM L-Gln and 100 units/ml penicillin-streptomycin. BRU medium used to grow H1-HeLa cells is S-MEM (Gibco BRL/Invitrogen Cat. No. 11380-037) supplemented with 0.1% pluronic acid F68 (BASF, Washington, N.J.), 0.1 M NEAA, 4 mM L-Gln, 100 units/ml penicillin-streptomycin, and 10% heat-inactivated calf serum. Phosphate-buffered saline (PBS) is from Gibco BRL/Invitrogen (Cat. No. 20012-027).

Enzymes and reagents. Restriction enzymes Apa I (cat No. R6361), Cla I (Cat No. R6551), Mlu I (Cat No. R6381), Taq DNA polymerase in buffer B (Cat. No. M1661), T7 RNA polymerase (Cat No. P2075), recombinant RNase inhibitor RNasin (Cat No. N2515), oligonucleotide triphosphate mix (rNTP) (Cat. No. E600), and deoxyoligonucleotide triphosphate mix (dNTP) (Cat. No. U1420) were bought from Promega. DNA polymerase I, Large Fragment (Klenow) (Cat. No. M210S), and high concentration T4 DNA ligase (Cat. No. M-0202M) were bought from New England Biolabs (NEB). Reverse transcriptase Superscript II (Cat. No. 18064-022) was bought from Invitrogen. Shrimp alkaline phosphatase (SAP; cat. No. 108-138) was purchased from Boehringer Mannheim Biochemicals (BMB). Kits for DNA and RNA extraction and purification, QIAGEN Plasmid Mini Kit (Cat. No. 12123), QIAquick PCR Purification Kit (Cat. No. 28104), QIAquick Gel Extraction Kit (Cat. No. 28704), QIAamp Viral RNA Mini Kit (Cat. No. 52904) were bought from QIAGEN Corp. (Valencia, Calif.).

Antibodies. Human mAb 2F5 which binds to the HIV-1 gp41 amino acid sequence ELDKWA (Conley, et al. 1994; Purtscher et al. 1994; D'Souza, Milman et al. 1995; Trkola et al., 1995; McKeating et al., 1996; Purtscher et al., 1996; D'Souza et al., 1997; Kessler et al., 1997) was provided by Hermann Katinger from the Institute of Applied Microbiology at the University of Agriculture, Vienna, Austria. This antibody was used for immunoselection and antigenicity assays. MAb 17 which binds to the natural immunogenic site IA (NIm IA) of HRV14 (Sherry and Rueckert, 1985), was provided by Roland Rueckert from University of Wisconsin at Madison and was used for ELISAs of chimeric rhinoviruses. Horse-radish Peroxidase (HRP)-conjugated goat-anti-human IgG (Cat. No. 55252), HRP-conjugated goat-anti-mouse IgG (Cat. No. 55554), and HRP-conjugated goat-anti-guinea pig IgG (Cat. No. 55291) are from Cappel ICN (Irvine, Calif.) and were used in the ELISAs of chimeric viruses and immune sera.

Peptides. Biotin-conjugated LELDKWASL-NH₂, Ac-LELDKWASL-NH₂, and Keyhole Lympet Hemocyanin (KLH)-conjugated EQELLELDKWASL-NH₂, were bought from Multiple Peptide Systems (San Diego, Calif.). Ac-CNEQELLEKDKWADL-NH₂ (Lac-1), a constrained peptide with a lactam bridge between the underlined K and D residues, and Ac-CENEQLELDKWASL-NH₂ (Linear 1) peptides were synthesized and purified by Yu Tian, then a graduate student in the laboratory of John Taylor (Rutgers University, Piscataway, N.J.).

Cassette Mutagenesis

Design and construction of ELDKWA cassettes. VP2 was chosen as a target based on the stability and immunogenicity of a chimeric virus from a library displaying an HIV-1 V3 loop epitope bound by a disulfide bond (Zhang et al., 1999). The V3 loop library was designed upon searching the geometries of disulfide-bound loops in known protein structures and finding that there was an apparently suitable place for insertion of the foreign epitope between residues Ser 157 and Val 162.

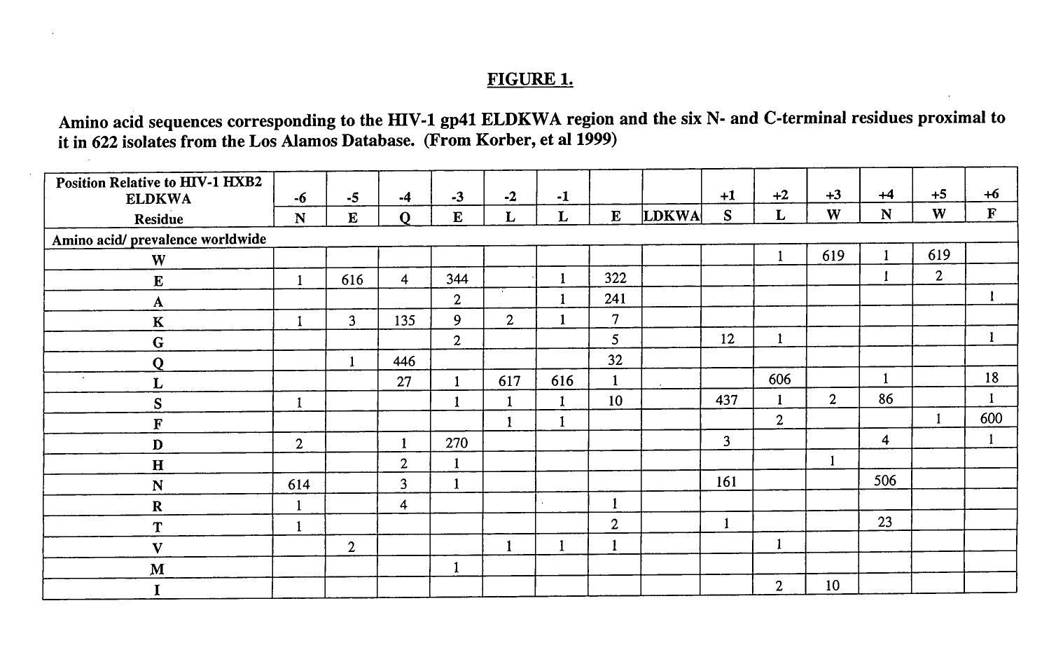

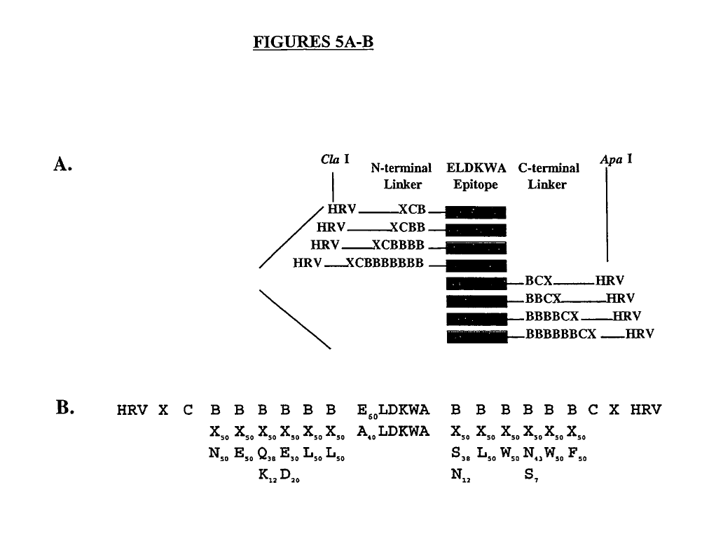

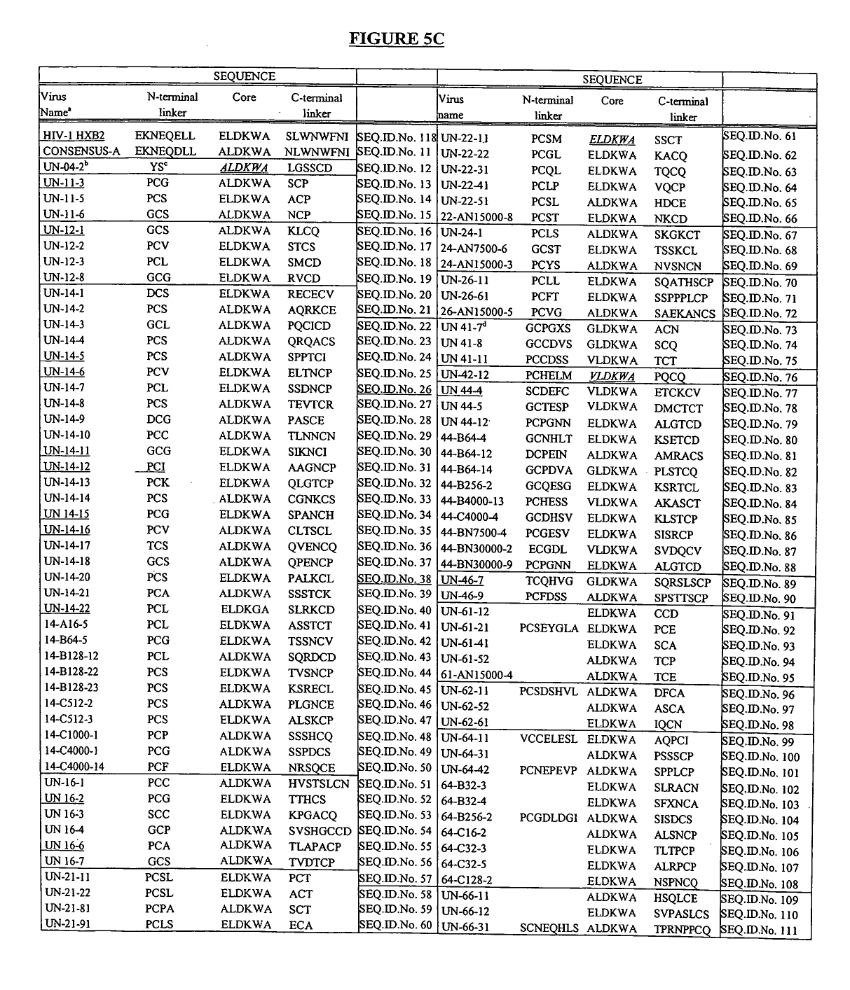

The ELDKWA cassettes contain cysteine in order to favor the formation of a disulfide bond that may provide more stable conformations and/or better presentations of the selected immunogenic epitope. The size and presentation of the loop may be important for viability of the virus and presentation of the epitope. Accordingly, the design considered display of the ELDKWA epitope at the HRV14 NIm-II site flanked on each side by 1 to 6 biased amino acids (encoding 50% HIV-1 residues and 50% a randomized residue), one cysteine, and one randomized linker residue. For the biased residues the design took into account the prevalence of the sequences flanking the ELDKWA epitope accounting for the more than 600 HIV-1 isolates belonging to different clades found in the Los Alamos Database at that time (Korber et al., 1998; FIG. 1). Flanking the ELDKWA/linker sequences, the cassette encoded 8 HRV residues on each side as well for its insertion between the ClaI and ApaI restriction sites (FIG. 5).

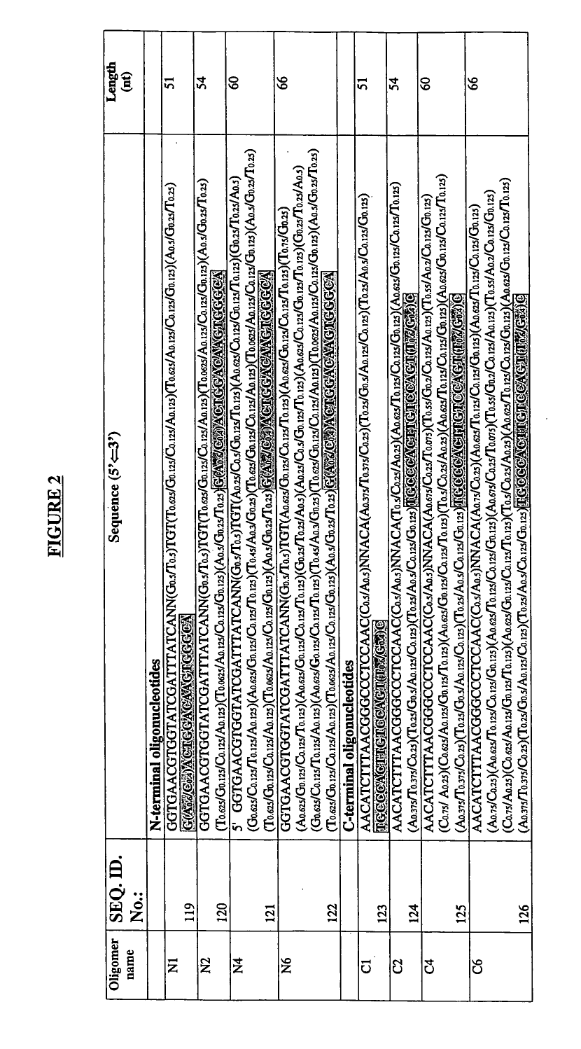

Four 5′ DNA oligomers encoding 1, 2, 4, and 6 biased N-terminal residues and the ELDKWA epitope and four 3′ DNA oligomers encoding 1, 2, 4 and 6 biased C-terminal residues were designed (FIG. 2) and synthesized (CyberSyn, Lenni, Pa.). The codon NN(G/C) was used to encode randomized amino acids, where N represents an equimolar mixture of each of the four nucleotides A, G, C, and T, and G/C represents an equimolar mixture of G and C at the wobble position. This would allow for each of the twenty commonly occurring amino acids to be encoded with a limited number of termination codons (Devlin et al., 1990) to minimize the production of non-infectious RNA transcripts during the in vitro transcription reactions. The biased amino acids of the HIV-1 core sequence required mixtures of phosphoramidites in the appropriate proportions so as to generate 50% NN(G/C) and 50% HIV-1 residues.

The four amino-terminal oligomers were each hybridized separately with each of the four carboxy-terminal oligomers in the region overlapping (E/A)LDKWA giving rise to 16 possible combinations. Thus, separate reactions were generated and handled as insert libraries of different sizes. The libraries of chimeric inserts, and subsequently of chimeric plasmids and HRV14:HIV-1 gp41 ELDKWA chimeras were named after the number of biased residues encoded at the N— and C-terminal sides of the ELDKWA epitope. Therefore, a library generated by an N-terminal oligomer encoding 2 biased residues, and a C-terminal one encoding 6 biased residues would be designated “26”. The 16 unique libraries were: 11, 12, 14, 16, 21, 22, 24, 26, 41, 42, 44, 46, 61, 62, 64, and 66. With these characteristics, a general description of the library design is shown in FIG. 5.

The oligonucleotides were received lyophilized and were resuspended into TE (10 mM Tris-HCl, 1 mM EDTA, pH 8.0) to a concentration of 2 μg/μl. Pairs of N— and C-terminal oligonucleotides were hybridized in separate reactions containing equimolar concentrations (20-50 nM) of each of the oligonucleotides, 2 mM Tris-HCl, pH 7.4, 5 mM MgCl₂, 10 mM NaCl, 1× Klenow DNA polymerase buffer, and 250 μM of each of the dTNPs (dATP, dCTP, dGTP, and dTTP) in a 200 μl reaction volume. The above mixture, except the polymerase was put into a Perkin Elmer Thermocycler heated at 95° C. for 3 minutes to denature the oligonucleotides, allowed to slowly cool down to 46° C. for a period of 4 hours during which time annealing of complementary regions could take place. After 10 minutes at 46° C., 5.73 units of Klenow DNA polymerase were added. The temperature was quickly decreased to 37° C. and held for 7 hours to allow the polymerase to fill in the single-stranded DNA regions, followed by a cool down to 4° C. The resulting cassettes were extracted with phenol:chloroform:isoamyl alcohol (volume ratios of 25:24:1, respectively), and then back-extracted with pyrogen-free Milli-Q H₂O. Following the extraction reaction, the double-stranded cassettes in the aqueous phase were precipitated with 0.3 M sodium acetate, pH 5.3, 8 mg glycogen (added as a carrier) and three volumes of absolute ethanol. The mixture was kept overnight, and then the precipitated DNA was centrifuged at 14,000 rpm in a tabletop Eppendorf microcentrifuge for 30 minutes at 4° C. The DNA pellets were washed once with 70% ethanol, left to dry for a few minutes, and then resuspended in pyrogen-free Milli-Q H₂O.

The cassettes were subsequently digested by 10 units of the enzymes ApaI and ClaI per μg of DNA in Promega Multicore Buffer, in a 200 μl reaction volume. The samples were incubated for 2 hours at 37° C., followed by a 20-minute incubation at 65° C. to inactivate the endonucleases, and a cooling down to 4° C. The resulting sticky ended cassettes were purified using the QIAquick PCR purification kit. The protocol of this is based on: (1) binding DNA of >70 bp to a positively charged resin, (2) washing it with a series of buffers (e.g., containing chelating agents to inactivate nucleases, and ethanol to remove contaminantes), and (3) elouting the column-bound DNA with a neutral pH buffer such as TE (Tris EDTA pH, 7.4, or Milli-Q H₂O) or the elution buffer provided in the kit (10 mM Tris-HCl, pH 8.5). To do so, five volumes of binding buffer PB were added to the digested cassettes and the mixtures were put into a QIAquick spin column and centrifuged for 60 seconds at 14,000 rpm in an Eppendorf tabletop microcentrifuge. The column was washed with 0.75 ml of wash buffer PE and centrifuged for 60 seconds. Finally the digested cassette was eluted with 30 μl of Qiagen elution buffer (10 mM Tris-HCl, pH 8.5). The purified DNA fragments were examined and quantified on polyacrylamide gels.

Preparation of plasmid for insertion of cassettes. Frozen DH 10B cells containing the p3IIST plasmid were inoculated into a 15 ml culture tube containing 5 ml of LB with 100 μg/ml Ampicillin. The preinoculum was incubated at 37° C. and shaken at 275 rpm for 5 hours. After that the preinoculum was poured into 250 ml of fresh LB-Amp and grown at 30° C. for 16 hours at 300 rpm. The cells were harvested by centrifugation in an SS-34 rotor at 10,000 rpm for 7 min at 4° C. and plasmid was obtained using the Dupont Prep alkaline lysis protocol (Sambrook et al., 1989). The resulting plasmid was digested with ApaI and ClaI enzymes in a reaction similar to the one used for generating the cassettes with cohesive ends. To avoid possible intramolecular re-ligation of the plasmid cohesive ends, the plasmid was subjected to an incubation with 10 units of Shrimp Alkaline Phosphatase/μg of plasmid for 1 hour, followed by a 65° C. 15 minute incubation period to inactivate the enzyme.

The resulting plasmid was loaded onto a 0.8% agarose gel and the band corresponding to the plasmid was cut from the gel and purified using the QIAquick Gel Extraction Kit (QIAGEN). This kit involves: (1) breaking up the agarose and liberating the DNA from it (with 10 minutes at 50° C. followed by the addition of isopropanol), (2) binding the DNA to a column, (3) washing it, and eluting it (using a principle similar to the one used in the PCR purification kit).

Bacterial Transformation And Isolation Of Plasmids Encoding Chimeric Virus Library Pools. An approximate molar ratio of 1 to 10 vector-to-insert was used for the ligation reaction, which included 2×10⁶ units of T4 DNA ligase/μg of DNA in a 500 μl volume. The ligase reaction was incubated for 16 hours at 16° C. The ligation reaction was purified by phenol:chloroform isoamyl alcohol (volume ratio 25:24:1 CHISAM) extraction and ethanol precipitation, then resuspended in 30 μl of Milli-Q H₂O.

Electroporation of DH 10B E. coli electroporation-competent cells was performed using a Gibco BRL Gene pulser. 3 μl of the ligation reaction (corresponding to approximately 100 ng of DNA) were added to 20 μl of cells and held on ice for one minute. The pulser settings used were: 400 V (voltage), 330 μF (capacitance), and 4000 Ohms (pulse controller). The cell-DNA mixture was placed in a 0.2 cm cuvette and voltage was applied. The electroporated cells were resuspended in 1 ml of SOC media and incubated at 37° C., shaking at 275 rpm for one hour. From this culture, samples were diluted to prepare 10⁻², 10⁻³, and 10⁻⁴ for plating into LB-Amp agar to assess transformation efficiency. The rest of the cultures were added to 100 ml of LB-Amp and incubated for 16 hours at 30° C. and 275 rpm. Recombinant plasmid was extracted using the Qiagen MiniPrep Spin kit from QIAGEN, which is based on the alkaline lysis method (Hendrickson and Hatfield, 1990). The colonies on the bacterial plates were counted to calculate the transformation efficiency for each pool of chimeric plasmids. Some colonies were picked and grown in LB-Amp to extract plasmids and analyze their size and sequence in order to make sure there were no sequence deletions.

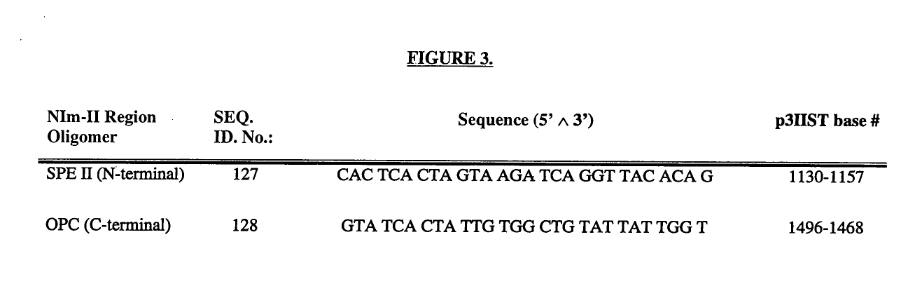

Sequencing Of Plasmids Encoding Chimeric Virus Library Clones. Individual bacterial colonies were grown in 5 ml of LB-Amp at 37° C. and 275 rpm for 12 hours. The plasmid DNA was extracted using the Qiagen MiniPrep Spin kit from QIAGEN (based on the alkaline lysis method; Hendrickson and Hatfield, 1990). Approximately 0.2 μg of plasmid DNA was added to a PCR reaction mixture containing 3 units of Taq Polymerase, 150 μM MgCl₂, 4 ng of each primer (OPC and SPE II), and 840 μM dNTPs (210 μM each). The PCR reaction was incubated in a Perkin Elmer Thermocycler with the following conditions: 30 cycles of 94° C. for 30 seconds, 60° C. for 30 seconds, 72° C. for 30 seconds; then an extension period of 7 minutes at 72° C. The resulting DNA was analyzed by electrophoresis in a 2% agarose gel. The band corresponding to a molecular weight of approximately 360 bp was cut from the agarose gel and purified using the QIAquick Gel Extraction Kit from QIAGEN. 60 ng of the purified PCR product and 6 pmol of SPE II primer (FIG. 3) were mixed and sent out (to GeneWiz, North Brunswick, N.J.) for sequencing.

In vitro transcription and transfection of HRV14:HIV-1 gp41 ELDKWA chimeric virus library constructs. The pools of recombinant plasmids were digested with the MluI restriction enzyme, which cuts the vector at the end of a long polyA tract that occurs immediately downstream of the HRV14 coding sequences. 5 units of MluI were used per μg of recombinant plasmid in a 500 μl reaction volume. The reaction was incubated for 2 hours at 37° C., then the enzyme was inactivated at 65° C. for 20 minutes and the digestion product was purified using the QIAquick PCR purification kit. The purified linear plasmid was eluted with 30 μl of elution buffer (EB). Pools of mutagenized plasmids were transcribed in vitro in a reaction containing 20 μl (approximately 1 μg) of plasmid, 20 units of T7 RNA polymerase, 7.1 mM DTT, 7.1 mM rNTP mix, and 24 Units of recombinant RNase inhibitor (RNasin), incubating at 37° C. for 2 hours. The full-length RNA transcripts generated were transfected into H1-HeLa cells by electroporation. 4-10 μl of the infectious RNA transcript were added to 10⁷ Hi -HeLa cells in 400 μl of D-MEM and then placed into a 0.4 cm Gibco electroporation cuvette. The Gibco Gene Pulser was set at 250V and 1180 μF. After pulsing the cells they were immediately pipetted into a 4 ml volume of medium M with 20% FBS and left at room temperature for 1 hour. The surviving cells were counted on the microscope and then diluted from 1/50 to 1/800 to assess transfection efficiency. The dilutions of pulsed cells were plated with unpulsed cells at ratios from 1/50 to 1/800 (pulsed cells/unpulsed cells) and a plaque assay was performed to count plaque formation. The number of plaques generated (where each viral plaque represents one transfectant) was multiplied by the number of surviving cells after electroporation in order to determine the total number of transfectants generated. The rest of the pulsed cells were plated (for harvesting the chimeric virus library) into 150 mm tissue culture plates with an equal proportion of unpulsed cells (9×10⁶ pulsed cells and 9×10⁶ unpulsed cells) in medium M supplemented with 20% FBS and incubated at 34.5° C. until an 80%-100% cytopathic effect (CPE) was observed.

Propagation And Isolation Of Immunoselected HRV14:HIV-1 Gp41 ELDKWA Chimeras. Immunoselected HRV14:HIV-1 gp41 ELDKWA chimeras were propagated in pools for screening their general antigenic characteristics. Individual chimeric virus clones were selected from the most antigenic pools produced from immunoselection. The viral clones were propagated and then characterized, and in the most favorable cases, purified for immunization of animals.

Plaque Purification Of Immunoselected Chimeric Viruses. The plaque assay consisted of infecting monolayers of H1-HeLa cells contained in tissue culture plates with serial dilutions of the immunoselected chimeric virus pools. Adsorption took place for 30 minutes at 34.5° C., 2.5% CO₂; after that, the infected cell monolayers were overlaid with 0.5% Noble Agar or low melting point agarose in PA medium supplemented with 10% FBS. The plates were incubated at 34.5° C., 2.5% CO₂ and observed under the microscope for the presence of plaques. The plates with the smallest number of plaques were chosen to isolate plaques from (in order to pick the plaques that were far away from one another). The plates were stained with a 1:2 solution of neutral red:PBS and incubated for 1 hour at 34.5° C., 2.5% CO₂. After the incubation, the liquid was extracted by aspiration and the plates were put into a light box to identify the plaques to be picked. The plaques were extracted by suction with Pasteur pipettes. The chunks of agar containing the viral plaques were put into 1.0 ml cryotubes containing 500 μl of PA medium. The tubes were frozen and thawed and vortexed three times in order to break the virus free from the agar and cells. The virus supernatant was recovered by centrifugation at 14,000 rpm in a tabletop Eppendorf microcentrifuge at room temperature, and then frozen at −80° C. for further use in propagation, characterization and sequencing experiments.

Propagation Of Viruses. Chimeric viruses or virus pools with low titer (<1×10⁵ PFU/ml, isolated from plaques or from immunoselection with peptide elution) were added to H1-HeLa cell monolayers in 60 mm dishes (containing 1.5×10⁶ cells). Chimeric viruses with titers higher than 1×10⁵ PFU/ml were propagated into H1-HeLa cell monolayers contained in 150 mm dishes (2.5×10⁷ cells/dish). The multiplicity of infection used for propagation of viruses with known titers was from 0.3-0.5 PFU/cell in 3 ml of medium M/1% FBS. The plates were incubated for one or two hours at 34.5° C., 2.5% CO₂ followed by the addition of either 3-5 ml (60 mm dishes) or 7 ml (150 mm dishes) medium M/1% FBS. The plates were incubated at 34.5° C., 2.5% CO₂ until CPE reached 80-100% (from 24-40 hours). Plates were frozen at −80° C. and then thawed at room temperature (˜23° C.) for three cycles, after which the cell debris was removed by centrifugation at 3500 rpm in a Sorvall H6000A rotor the virus supernatant was stored at −80° C. For chimeric virus clones of unknown titers ˜300 μl of the recovered supernatant from the plaque was used to infect H1-HeLa cell monolayers in a 60 mm plate. The plates were incubated and harvested in the same way as for viruses with known titers.

Titering Of Viruses. Chimeric virus pools and single clones were titered using plaque assays. Monolayers of H1-HeLa cells were infected with various dilutions of virus (10⁻³ to 10⁻⁸) depending on the expected titer. Four to six dilutions were plated in duplicate on H1-HeLa cell monolayers (170 μl virus dilution, 7.5×10⁵ cells, 60 mm tissue culture plate, or 50 μl virus dilution, 1.5×10⁵ cells/well, 12-well tissue culture plate). After a 30 minute adsorption period at 34.5° C., 2.5% CO₂, the plates were overlaid with PA medium containing 10% FBS and 0.5% noble agar or agarose (5 ml/60 mm plate or 1 mL/well for 12-well plates). After an incubation period of 72 hours at 34.5° C., 2.5% CO₂, the plates were fixed with 10% formaldehyde and stained with 0.1% crystal violet. Plaques were counted to determine the chimeric virus titers.

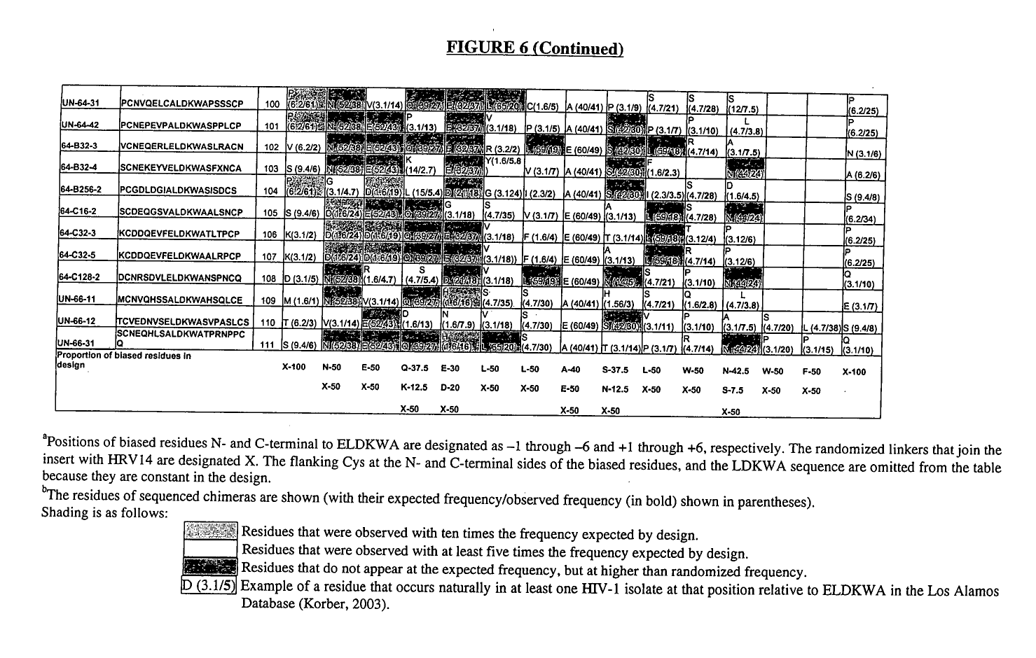

Partial Purification Of HRV14:HIV-1 Gp41 ELDKWA Chimeric Virus Library Pools. After a few viral clones from the chimeric virus libraries were isolated and sequenced to make sure that these contained the ELDKWA inserts and that there were no unwanted deletions of HRV 14 residues, pools of chimeric viruses were propagated in monolayers, and then partially purified. It was decided to purify virus pools before any other characterization procedure because in past experiences some chimeras that had been characterized turned out to be lost during the purification protocol. To avoid this risk, it was decided to work with pools of pre-purified viruses. The partial purification consisted in spinning down the cell lysates obtained after virus propagations in an SS-34 Sorvall rotor at 12,000 rpm for 15 minutes at 15° C. to pellet most of the cell debris left on the lysate. The supernatant was then poured into 45Ti ultra-centrifuge tubes and underlaid with a 30% sucrose cushion to minimize mechanical damage to the virus and to help with purification of the virus after centrifugation at 45,000 rpm for 2 hours at 15° C. The viral pellet was resuspended in 200-250 μl of 10 mM Tris-HAc, pH 7.4/0.05 M NaCl, frozen in liquid nitrogen and stored at −80° C. The virus obtained by this method still can contain cell-derived ribosomes, and it is not quantifiable by optical density (OD) readings. The yields were estimated by a plaque-forming assay, and quantities were estimated by using the average of equivalent plaque forming units (PFU) to mgs obtained for previously fully purified chimeras. Immunoselection Of HRV14:HIV-1 Gp41 ELDKWA Libraries Using mAb 2f5. The immunoselection conditions were varied in order to explore several approaches that might lead us to enrich for chimeric viruses with the ELDKWA epitope transplanted in an immunologically relevant conformation. In previous work performed with chimeric rhinovirus libraries (Resnick et al., 1995; Smith et al., 1998; Bayman et al., in preparation), a direct immunoselection approach with one or more antibodies was used. Alternative approaches were tested that might yield pools richer in immunogenic chimeras (FIG. 7).

Immunoselection. The chimeric rhinovirus pools were immunoselected for their ability to be trapped by the mAb 2F5, which recognizes the ELDKWA epitope. It was expected that the chimeras best recognized by this antibody might contain a conformation of the epitope similar to the one that elicited the production of 2F5. The immunoselection involved immobilizing 100 μl of 2F5 (at 1 μg/ml in 50 mM sodium borate, pH 8.5) in a plastic 96-well microtiter plate (NUNC). After an overnight incubation at 4° C., the plate was washed and blocked with 3% gelatin dissolved in PBS. The plate was incubated at 37° C. for 1 hour and washed six times with PBS/0.05% Tween 20 (PBS-T; 300 μl/well). Then 3×10⁵ PFU of the chimeric virus pool were added per well. The plate was incubated for 2 hours at 37° C. and then washed again six times with PBS-T and three times with PBS. Finally 2×10⁴ H1-HeLa cells in medium M/10% FBS were added per well. The plate was incubated at 34.5° C. and 2.5% CO₂ for up to 72 hours and cells were harvested when 80-100% CPE was observed. The cells were lysed by freeze thawing and the virus was recovered from the lysate by centrifugation of the cell-pellet at 3500 rpm in a Sorvall SS-34 rotor.

Competitive immunoselection. This approach was used with the purpose to immunoselect viruses with higher stringency, and enrich for chimeras that had better antigenicity (ability to be recognized by antibody, in this case 2F5). This assay is based on the ability of the chimeric viruses to bind to immobilized 2F5 in the presence of a competing peptide with the sequence LELDKWASL, which was shown previously to bind with high affinity to 2F5 (Tian et al., 2002). The assay was performed as described in with the addition of the competing peptide Ac-LELDKWASL-NH₂ at the same time the chimeric virus was added to the plate. The peptide concentrations used were from 2 to 4,000 pmol/well. Subsequent rounds of increasingly stringent immunoselection (lowering the amount of immobilized 2F5, and increasing the amount of the competing peptide) were performed (FIG. 7) and samples of the viruses generated after each immunoselection round were analyzed for their ability to be recognized by mAb 2F5 (using direct ELISAs and microtiter neutralization assays).

Immunoselection using peptide elution. Some virus pools were subjected to a first or second round of immunoselection using peptide elution (FIG. 7). This variation of the immunoselection approach involved a modification of the last step of immunoselection where instead of adding H1-HeLa cells to “elute” the chimeric viruses bound to 2F5, the Ac-LELDKWASL-NH₂ peptide was added at concentrations ranging from 75 to 300 μM in PBS. This variation was implemented to avoid the possibility that the chimeric viruses that most tightly bind 2F5 might be neutralized by this antibody and unable to propagate in H1-HeLa cells, therefore, resulting in their loss during cell elution. In this case, it was expected that the high concentrations of LELDKWASL peptide should be able to dissociate the virus-2F5 complexes, and the free virus would be rescued after a 2-hour incubation period at 37° C. After this incubation, the virus-peptide mixture was recovered by pipetting and the virus recovery could be quantified with a virus plaque assay.

Subsets of virus pools underwent different combinations of these variations of the immunoselection approach: pool I (FIG. 7), which consisted of libraries 12, 14, 16, 42, 44, 46, and 64, went through three rounds of competitive immunoselection; pool II, consisting of libraries 14 and 44, was subjected to one round of competitive immunoselection, and then was subjected to either a second round of competitive immunoselection, or a round of immunoselection without peptide competition that included elution of bound virus by peptide. Pool III, consisting of libraries 11, 12, 14, 16, 21, 22, 24, 26, 41, 44, 61, 62, and 64, was subjected to either two subsequent competitive immunoselection rounds, or to one non-competitive immunoselection round with peptide elution. The peptide concentrations used for both competition and elution of Pool III viruses were slightly less than the ones used on the other pools in order to determine conditions that were sufficiently stringent to select for the most antigenic chimeras, but at the same time trying to conserve some diversity among the immunoselected clones.

Sequencing of Viral RNA. Viral RNA was extracted from supernatants of chimeric virus clones (obtained after freeze-thawing and centrifuging cell-lysates obtained from isolated viral plaques) using the QIAamp Viral RNA Purification Kit from QIAGEN. After treating the viral supernatant (140 μl) with a buffer containing carrier RNA and RNase inhibitors (560 μl), the samples were incubated at room temperature for 10 minutes, after which 560 μl of 96-100% ethanol were added. The mixture was put into a QIAamp spin column and centrifuged at 10,000 rpm in an Eppendorf tabletop microcentrifuge. The column was then washed with a buffer containing ethanol and other unspecified components that wash away proteins and other contaminants. Then the RNA was eluted with pre-heated RNase-free water (23 μl). 23 μof the purified RNA were added to a reverse transcription mixture containing 40 units of Superscript II reverse transcriptase (Invitrogen), reverse transcriptase buffer, 8.75 mM DTT, 150 μM dNTPs (37.5 μM each), 0.5 μg of OPC primer (FIG. 3), and 20 units of RNasin (recombinant RNase inhibitor). The reverse transcription reaction was incubated at 42° C. for 60 minutes, followed by a 70° C. enzyme inactivation period of 15 minutes and a 94° C. denaturation period of 3 minutes. The tubes were cooled down on ice and 60 μl of a PCR mixture containing 3 units of Taq Polymerase, 150 μM MgCl₂, 4 ng of each primer (OPC and SPEII), and 840 μM dNTPs (210 μM each) were added. The PCR reaction was incubated in a Perkin Elmer Thermocycler with the following conditions: 30 cycles of 94° C. for 30 seconds, 58-60° C. for 30 seconds, 72° C. for 30 seconds; then an extension period of 7 minutes at 72° C. The resulting DNA was analyzed by electrophoresis in a 2% agarose gel. If there was a single and relatively clean band of appropriate size, the PCR product was purified using the QIAquick PCR purification kit. When there were more bands of other molecular weights, the desired band was cut from the agarose gel and purified using the QIAquick Gel Extraction Kit from QIAGEN. 60 ng of the purified PCR product and 6 pmol from the SPE II primer (FIG. 3) were sent to GeneWiz (North Brunswick, N.J.) where sequencing of the samples was done.

Antigenicity Assays

Direct ELISA For Pools And Single Clones. Enzyme-linked immunosorbent assays (ELISAs) were performed on all partially purified chimeric virus pools to monitor virus binding to 2F5 as a function of insert size and immunoselection conditions. Direct ELISAs were subsequently used to characterize individual clones for their ability to bind 2F5.

96-well NUNC plates were coated with 0.1 μg/well of 2F5 in 50 mM sodium borate, pH 8.5. The plates were kept at 4° C. overnight, then blocked with a solution of PBS/3% gelatin for 1 hour at 37° C. After washing the plates six times with PBS/0.05% Tween 20 (PBS-T) for 1 minute each time, 6 serial 2-or 4-fold dilutions (from 1×10⁷to 1.9×10⁴PFU) of virus in PBS-T were added to the plate in duplicate. The virus was incubated on the plates for 2 hours at 37° C., followed by washing 6 times. Then 100 μl of a 4.2 μg/ml solution of murine mAb 17 which recognizes the NIm-IA epitope on HRV14, and also on chimeras, as this site has not been engineered in this study) were added to each well. After a one-hour incubation period at 37° C. and 6 washes, a secondary antibody (horseradish peroxidase-conjugated goat-anti-mouse IgG) was added in a 1/1000 dilution followed by another 1-hour incubation period at 37° C. The plates were washed 6 times and bound antibody was detected by incubating all wells for 5-30 minutes with 100 μl of a solution containing tetramethyl benzidine (TMB; Boehringer Manheim), a chromogenic substrate in the presence of hydrogen peroxide (H₂O₂). OD values at 450 nm were read using a DeltaSoft plate reader. The OD values were plotted against the virus dilution, and the virus dilutions corresponding to an OD of 0.5 were determined by interpolation of points in the linear part of the curve.

Microfiter Neutralization Assays For Chimeric Virus Pools And Clones. A second assay used to evaluate the antigenicity of the chimeric viruses was based on their sensitivity to neutralization by mAb 2F5. It was assumed that the viruses more sensitive to neutralization by 2F5 were also the ones that displayed the ELDKWA epitope in the most antigenic conformations. Pools of unselected viruses, as well as individual immunoselected clones were characterized using this assay. This assay was also used to evaluate the ability of the serum elicited by certain chimeras to neutralize the homologous virus.

1×10⁴ PFU of virus were added to 50 μl of various serial dilutions (4-0.000123 μg/ml) of mAb 2F5 in a 96-well NUNC plate. After a one-hour incubation at 34.5° C. and 2.5% CO₂, 1×10⁴ H1-HeLa cells were added per well. The plate was incubated at 34.5° C. and 2.5% CO₂ until viral CPE reached 100% in control wells containing no antibody (from 40 to 48 hours depending on the virus). 15 μl of a 5 mg/ml solution of 3-4,5-dimethylthiazol-2-yl-2,5 diphenyltetrazolium bromide (MTT) were added to each well and incubated at 34.5° C., 2.5% CO₂ for 1.5 hours. The reaction was stopped by adding 150 μl of a solution containing 50% N,N-dimethyl formamide and 20% sodium dodecyl sulfate (SDS). The plates were left at room temperature for 30 minutes to allow the MTT insoluble dye product to dissolve. MTT is a dye that is yellow in acidic solutions but it is converted to an insoluble purple formazan by cleavage of the tetrazolium ring by active mitochondrial dehydrogenases. Therefore, this assay measures the percentage of surviving H1-HeLa cells (which have active mitochondrial dehydrogenases and can cleave MTT) at the moment the MTT is added. The control wells with virus but no antibody represent 100% cell death (background control), and the control wells containing only cells (with no virus added) represent 100% cell viability. OD₅₇₀ values were determined and expressed as a percentage of the average absorbance in the cell control wells (subtracting background absorbance from all wells). Titers are expressed as the reciprocals of the dilutions of the antibodies that reduced cell death by 50%.

Virus Purification. Immunoselected chimeric virus clones that were selected to use for immunization of guinea pigs were partially purified using the protocol described on. Two immunoselected chimeric virus clones with some of the most promising antigenic properties, as well as favorable growth characteristics were selected for crystallization experiments. These two chimeric viruses, 14-C4000-1, and 44-C4000-4, underwent a full purification process in order to be suitable samples for crystallization experiments. It was also necessary to fully purify wild-type HRV14 (in order to couple it to a CNBr-activated sepharose column what would ultimately be used to deplete some guinea pig sera samples of antibodies directed against HRV14).

HRV14 and HRV14:HIV-1 gp41 ELDKWA chimeras were purified using the protocol described by Zhang et al. (1993), using differential high speed centrifugations, with some modifications. The clarified cell-lysates were treated with 50 mM MgCl₂ and 10 μg of Worthington DNAse I per ml of lysate. After a 30-minute incubation at room temperature, the DNAse was inactivated with 100 mM Na₂EDTA, pH 9.5, followed by 1% N-lauryl sarcosine to help dissolve residual lipids. The lysate was centrifuged at 12,000 rpm in a Sorvall SS34 rotor for 15 minutes at 15° C. Then the supernatant was underlaid with a 30% sucrose cushion and centrifuged at 45,000 rpm (240,000×g) in a Beckman 45Ti rotor for 2 hours at 15° C. The viral pellet was resuspended in 20 mM Tris-HAc, pH 7.4 (containing 0-0.2 M NaCl depending on the solubility characteristics of the virus) and overlaid onto a 7-45% sucrose gradient in 10 mM Tris-HAc, pH 7.4, and centrifuged at 35,800 rpm (160,000×g) in a Beckman SW40 rotor for 1.5 hours at 15° C. Fractions were collected in an ISCO Density Gradient Fractionator, and the virus-containing fractions were identified by their absorbance at OD₂₆₀. The fractions containing virus were diluted in ˜45 ml 10 mM Tris-HAc, pH 7.4, 0-0.2 M NaCl and pelleted at 45,000 rpm (240,000×g) in a Ti45 Beckman rotor for 2 hours at 15° C. The viral pellets were redissolved in 150-250 μl of 10 mM Tris-HAc, pH 7.4, 0-0.2 M NaCl and their concentration was calculated by their optical density. The virus concentration was brought to 0.4 mg/ml, frozen with liquid nitrogen and stored at −80° C. in 250 μl aliquots.

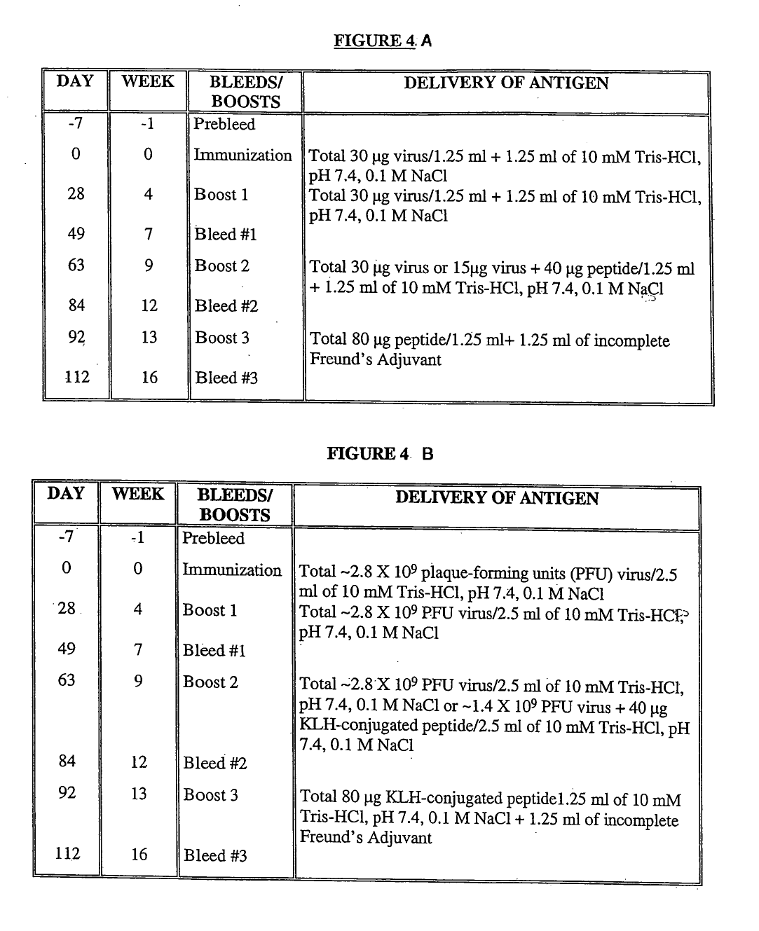

Immunization Of Guinea Pigs. Three young guinea pigs (Cocalico Biologicals, Reamstown, Pa.) were immunized subcutaneously with each immunoselected virus clone, approximately 3×10⁹ PFU (roughly ˜30 μg) or, in a few cases, with cocktails containing two or three viruses (totally ˜3×10⁹ PFU) at weeks 0, 4, and 9. In some cases an additional immunization with 80 μg of an ELDKWA-based peptide conjugated to keyhole limpet hemocyanin (KLH) was added on week 9 and/or 13 FIG. 4). Preimmune sera were collected one week prior to the first immunization. Equal volumes of virus and 10 mM Tris-HCl pH 7.4 (2.5 ml total volume) were used in all immunizations injecting in 4-6 places; immunizations with peptide were done with an equal volume of incomplete Freund's adjuvant. Serum samples were collected at weeks 7 and 12 (and 16 when a 13-week immunization had been applied. As observed later, the amount of immune serum was very limiting for its use in all the characterization assays desired. In response, the guinea pigs immunized were completely exanguinated at week 12 or 16 to obtain larger amounts of immune sera (10-15 ml). All guinea pig sera were tested in direct ELISAs for their ability to bind a peptide with the sequence LELDKWASL.

Protein A Fractionation Of Guinea Pig Antisera. Some of the guinea pig sera that had an outstanding (compared to the rest of the samples) ability to recognize the LELDKWASL peptide in a direct ELISA were purified with a protein A-sepharose affinity column in order to isolate and quantify the IgG fraction of the sera and to further characterize its peptide binding ability relative to that of mAb 2F5.

The approximate binding capacity of protein A is 10-20 mg of immunoglobulin (Ig) per ml of wet beads. It is estimated that immune sera from rodents contains about 10 mg of Ig per ml. The volumes of the guinea pig serum samples ranged from 0.3 to 6 ml (depending on availability); therefore, the volume of the columns used varied between 1 and 5 ml. The columns were equilibrated with 1.5 M glycine, 3M NaCl, pH 8.9 (buffer A). The serum sample was directly applied to the column with an equal volume of buffer A and mixed for 1 hour at room temperature. The liquid was removed and the beads were washed with more buffer A to remove unbound material and the IgG fraction was eluted by the addition of 100 mM citric acid, pH 3. The eluate was immediately neutralized by receiving it in a tube containing 1 M Tris-HCl, pH 8.0. The IgG concentration was estimated by reading the OD₂₈₀. The samples were buffer-exchanged and concentrated using Centricon tubes with a 30-kDa membrane pore size. The IgG concentrations were brought up to 10-20 mg/ml in 20 mM Tris-HCl pH 8.0/50 mM NaCl depending on the total amount of sample available, and stored at −80° C. in 150-200 μl aliquots.

Direct And Competitive ELISAs To Assess The Reactivity Of Guinea Pig Sera And 2F5 To ELDKWA-Based Peptides. All guinea pig serum samples were analyzed by a direct ELISA for their ability to recognize a peptide with the sequence LELDKWASL. The IgG fraction of selected samples with the best binding affinities to LELDKWASL were isolated, quantified and again tested for their binding ability to LELDKWASL peptide, this time compared with similar concentrations of mAb 2F5. For some of the IgG fractions available in sufficient quantity, competitive ELISAs were performed in which either one of two peptides designated Linear-1 (Ac-CENEQELLELDKWASL-NH₂) or Lac-1 (Ac-CNEQELLEKDKWADL-NH₂, with a lactam bridge connecting the underlined K and D residues) competed with LELDKWASL for binding of the IgG fractions. This was done as a complementary assay to assess the preference or specificity of the sera for any of these peptides, since the Linear-1 and Lac-1 have higher affinities to 2F5 than does LELDKWASL (Tian, 2003).

Direct ELISA

Streptavidin-coated 96-well plates preblocked with Reactibind buffer (Pierce) were incubated with 100 μl of 0.4 μg/ml Biotinylated LELDKWASL (B*LELDKWASL) peptide in PBS/0.5% BSA and kept overnight at 4° C. The plates were washed six times (1 minute each time) with PBS-T and serial dilutions of guinea pig serum or purified IgG were added to duplicate wells and incubated for 2 hours at 37° C. The plates were washed six times and a secondary antibody was added (horse-radish peroxidase-conjugated goat anti-guinea pig IgG for guinea pig sera, or horse-radish peroxidase-conjugated goat anti-human IgG for 2F5 both diluted 1/1000 in PBS-T). After a 1-hour incubation period at 37° C., bound antibody was detected by incubating all wells for 5-30 minutes with 100 μl of a solution containing tetramethyl benzidine (TMB) in the presence of hydrogen peroxide (H₂O₂). OD values at 450 nm were read using a DeltaSoft plate reader. The OD values were plotted against the serum/IgG dilution, and the dilutions corresponding to an OD₄₅₀ of 0.5 were determined by interpolation of points in the linear part of the curve and compared with the values obtained for 2F5.

Competitive ELISAs

Streptavidin-coated 96-well plates preblocked with Reactibind buffer (Pierce) were incubated with B*LELDKWASL as described in. In 96-well microtiter plates a fixed concentration of guinea pig IgG or 2F5 (corresponding to the concentration that resulted in an OD₄₅₀ of 0.5) was pre-incubated at room temperature for 2 hours with sequential 2- or 4-fold dilutions (2560-1.25 pM) of competing peptide, either Linear-1 or Lac-1. After washing the peptide-coated plates six times with 25 mM Tris-HCl, pH 7.2,150 mM NaCl, 0.1% BSA, 0.05% Tween-20 (TBS buffer), 100 μl of the preincubated IgG/competing peptide mixture was added to the plates and incubated for 2 hours at 37° C. The plate was washed six times with TBS and horseradish peroxidase-conjugated secondary antibody (goat anti-human or goat anti-guinea pig IgG in 1/1000 dilution) was added to the plates, bound antibody was detected. The IC₅₀ for each competing peptide and IgG combination was designated as the concentration of competing peptide that caused 50% inhibition of binding of a non-saturating amount of IgG to biotinylated LELDKWASL (determined for each serum sample by the curves obtained in the direct ELISA).

Neutralization Assays. Selected sera and IgG samples were assayed for their ability to neutralize HIV-1 TCLA and primary isolates in vitro. The neutralization assays were performed at Virologic (South San Franciso, Calif.) by Dr. Terri Wrin in the group of Dr. Christos J. Petropoulos. The assay was derived from a recombinant virus assay initially developed to measure antiviral drug resistance described in (Richman et al., 2003).

HIV genomic RNA was isolated from virus stocks of plasma and cDNA was synthesized by standard reverse transcription. Env DNA was amplified by PCR with forward and reverse primers containing unique PinAI and MluI restriction sites. The amplified DNA was digested and ligated into the corresponding restriction sites of pCXAS expression vector, which contains the CMV immediate-early promoter-enhancer to express the insert in transfected cells. Competent E. coli (Invitrogen) were transformed with recombinant vectors and pCXAS-env DNA was recovered from bacterial cultures. HEK293 cells were transfected with pCXAS-env libraries and an HIV genomic vector (without env) that contains a firefly luciferase indicator gene, leading to the production of viral particles containing the imported HIV envelope proteins. The recombinant viruses (pseudotyped with patient virus envelope proteins) were incubated for 1 hour at 37° C. with serial 4-fold dilutions of serum or IgG samples (antibody). U87 cells that express CD4 and the co-receptors CCR5 and CXCR4 were inoculated with various pseudotype virus-antibody dilutions and pseudotype virus replication was measured after a 72-hour incubation period by measuring luciferase activity expressed by the viruses. Neutralization was determined as % inhibition={1−[luciferase(+)Ab/luciferase(−)Ab]}×100, where the luciferase activity at each antibody dilution is compared to that of an antibody negative control. IC₅₀s were designated as the reciprocal of the antibody dilution conferring 50% inhibition of luciferase activity. The resultant IC₅₀ values were rounded to the closest 5 (i.e., a calculated IC₅₀ of 67 is reported as 65).

Trypsin Digestion. The design of the library included the insertion of cysteines flanking the ELDKWA insert of the chimeric viruses to promote the formation of a disulfide bond. It was decided to test at least one of the immunoselected chimeras with good antigenic and immunogenic characteristics for the presence/absence of a disulfide bond. The assay requires fully purified virus, and the availability of it was very limited as its use was reserved mostly for crystallization experiments, therefore it was only done on chimera 14-C4000-1. The assay is based on a trypsin digestion: trypsin cleaves R—X and K—X bonds, the chimeric viruses contained the K—X cleavage site in the middle of the inserted ELDKWA cassette, and digestion would lead to the cleavage of VP2 into two fragments with approximate molecular weights of 18 and 11 kDa. On the other hand, if a disulfide bond existed in the chimeric virus insert, even after cleavage, the VP2 protein would migrate as an intact protein (approximately 30 kDa) upon electrophoresis under non-reducing conditions.

Purified virus at a 3 mg/ml concentration was treated with 0.2 or 0.4 mg/ml trypsin (bovine pancreatic, SIGMA, T1426) dissolved in 1 mM HCl. The reaction was incubated in 10 mM Tris-HAc, pH 7.4 at 34.5° C. The digested samples were subsequently heated at 95° C. for 1 minute in SDS sample buffer (20 mM Tris-HCl, pH 6.8, 1% SDS, 1% glycerol and 0.02% bromophenol blue) with or without 2.5% 2-mercaptoethanol. The samples were loaded on an 8-25% linear polyacrylamide gradient gel (Phastgel, Pharmacia) for analysis.

Crystallization Of Chimeric Viruses From ELDKWA Libraries. Chimeric viruses 14-C4000-1 (insert sequence: PCGALDKWASSPDCS), and 44-C4000-4 (insert sequence: GCDHSVELDKWAKLSTCP) were selected for crystallization experiments (prior to obtaining the HIV-1 neutralization data) due to their favorable antigenic properties, their relative ease of propagation and purification, and the fact that they presented different sizes of linker residues and serovariants of the E/A residue.

The material used for crystallization consisted of approximately 300 μg of purified chimeric virus at a concentration of 0.4 mg/mi. Some of this material was previously frozen at −80° C. The virus was concentrated by ultracentrifugation in a Beckman 50.2 Ti rotor at 38,000 rpm (180,000×g) for 2 hours at 15° C. The virus pellet was resuspended in 10 mM Tris-HAc, pH 7.4, 0.05 M NaCl to a concentration of 5 mg/ml (60 μl volume). The resuspended pellet was centrifuged for 5 minutes at 14,000 rpm in an Eppendorf microcentrifuge. All but approximately 1 μl of solution was removed with a micropipette and transferred to a fresh microcentrifuge tube. Hanging drops were set up with this virus sample.

Crystallization conditions were set up based in three basic schemes:

1) a screening set up previously for several members of an HRV14:HIV-1 V3 loop library previously generated (Smith et al., 1998) that led to the structure determination of one of these chimeras, MN-III-2 (Ding et al., 2002). The screening conditions contained the virus solution (5 mg/ml 10 mM Tris-HCl, pH 7.4), 0.6-2.0 M ammonium formate, and 0.05-0.15 M HEPES buffer, pH 7.1-7.8.

2) an alternate screening was performed with a different set of ELDKWA-based chimeras that contained the virus solution, 0.2-1 M sodium/potassium tartrate, and 0.075-0.15 M HEPES buffer, pH 6.6 to 7.8.

3) conditions based on the work of Arnold et al. (1984) containing the virus solution and 10 mM Tris-HCl, pH 7.2, 20 mM CaCl₂, and 0.5% PEG 8000.

The volume of the hanging drops was 2 or 4 μl (mixed with a similar quantity of reservoir buffer) for screening, and it was increased to 8 μl for screening with a narrower set of conditions with the intention of getting larger crystals. 600 μl of the reservoir solution were placed in the wells of 24-well Linbro plates (ICN Biomedicals). The virus sample was placed on a plastic coverslip and mixed with an equal volume of reservoir solution. The crystals were grown at room temperature (22-23° C.) for 1 to 5 days and then placed at 4° C.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### EXAMPLES

### Example 1

### Generation of HRV14:HIV-1 GP41 ELDKWA Libraries

Combinatorial libraries were generated in which immunogenic HIV sequences were presented on the surface of HRV, connected via linkers with variable lengths and sequences, leading to chimeric viruses displaying the HIV immunogens in myriad conformations, some resembling those of HIV. Immunoselection and competitive immunoselection procedures were used to enrich for viruses that best bind immobilized, neutralizing anti-HIV antibodies. Promising viruses were used to immunize guinea pigs and the sera and/or IgG were tested for HIV-neutralizing activity.

Immunoselected chimeric viruses were identified that were able to elicit anti-HIV neutralizing responses in vitro. Some of the purified anti-ELDKWA IgGs possessed binding activities nearly equal to those of the strongly neutralizing ELDKWA-binding mAb 2F5. In some cases, roughly 50% of the IgG present in the sera was reactive with ELDKWA peptides.

HRV14 can be used to display HIV epitopes on its surface and produce prolific and effective humoral immune responses targeted to the transplanted epitopes. Immunoselection with combinatorial libraries presenting the ELDKWA epitope enriched for highly immunogenic chimeras, some of which elicited HIV-neutralizing antibodies. Many antibodies are reactive with HIV epitopes but unable to neutralize HIV, suggesting that: (1) antibody binding to the HIV epitope may be too limited or inappropriate to effect neutralization; (2) the antibody may be blocked from accessing the epitope; or (3) the antibody may require binding to part of the neutralizing epitope that is missing in the immunogenic construct. Antibodies capable of neutralizing HIV-1 might require extremely uncommon physical features, such as unusually long CDR H3 loops (as in the cases of mAbs 2F5 and IgG1 b12) or unusual interdomain relationships (as in the cases of IgG1 b12 and mAb 2G12).

The ELDKWA sequence (SEQ. ID. NO.: 1) of HIV-1 gp41 is the epitope to which the potently and cross-neutralizing human mAb 2F5 binds (Muster et al., 1993). A set of libraries displaying the ELDKWA epitope and its serovariant, ALDKWA (SEQ. ID. NO.:2), was produced, to present the epitope in numerous conformations, some of them potentially mimicking the natural presentation that elicited the production of mAb 2F5.

1. Scheme for Producing and Characterizing 16 HRV14:HIV-1 gp41 ELDKWA Libraries

The site chosen for engineering HRV14's NIm-II site has proven to give rise to viable chimeric viruses presenting foreign immunogens (Resnick et al., 1994; Smith et al., 1998; Zhang et al., 1999). However, it is important to take into account that a majority of the chimeric constructs generated by the library design are not likely to lead to the production of viable viruses, and a large portion of the live chimeric viruses will not display the foreign epitope in an immunogenic conformation similar to the one that elicits the production of neutralizing antibodies; a proportion of the library generated displays the foreign epitope in the desired conformation which mimics the natural one (FIG. 7). The advantage of working with the combinatorial library approach is that pools containing millions of chimeric viruses can be generated and screened for their immunogenic characteristics by making use of several characterization assays, narrowing the sample size and enhancing identification of the most immunogenic candidates. Introducing a foreign epitope in the capsid of HRV14 can lead to: 1) a disruption of the viral structure that renders the virus inviable, 2) a viable chimeric virus that presents the foreign epitope in a conformation that does not resemble that of the HIV-1 native immunogen, 3) a viable chimeric virus that presents the foreign epitope in a conformation similar to that of the native one.

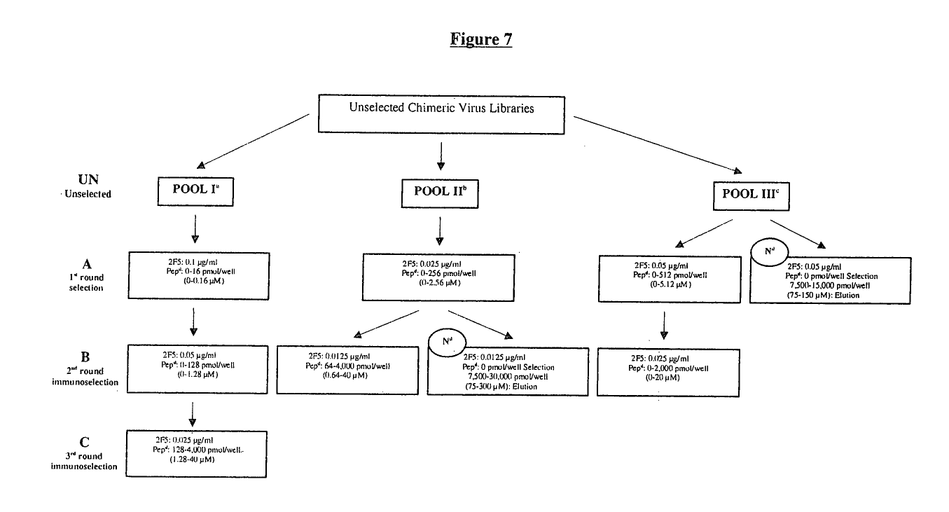

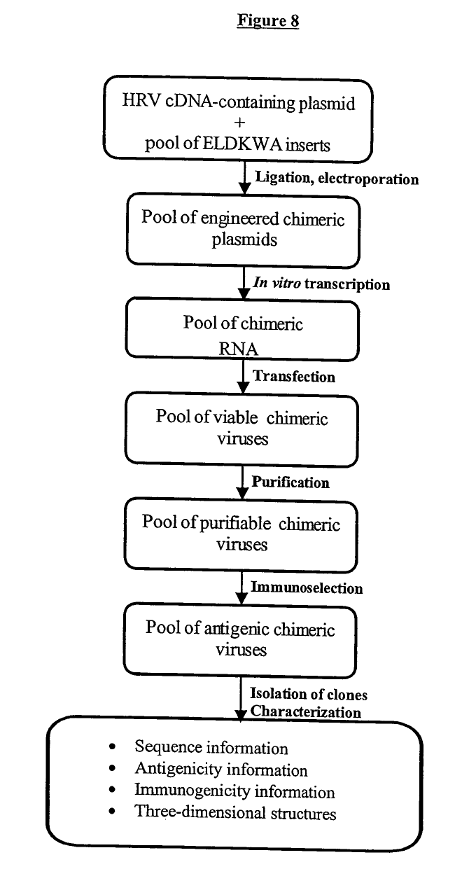

The general approach used to get to this point is shown in FIG. 8. The products of ligation reactions of p3IIST plasmids and mutagenic cassettes were transformed into bacteria, generating pools of chimeric plasmids. The plasmids were transcribed in vitro in order to generate infectious chimeric RNAs. A subset of RNAs generated viable chimeric viruses upon transfection of H1-HeLa cells. These pools of viable chimeric viruses were subjected to a series of partial purification steps. The next challenge was to identify chimeric viruses that presented the foreign construct in an immunogenic conformation. Accordingly, the pool of purified chimeric viruses was subjected to immunoselection with human mAb 2F5. Human mAb 2F5 recognizes inserted epitopes, thereby yielding pools of antigenic chimeric viruses, which were later characterized for their antigenicity, immunogenicity, and sequence. Based on this data, chimeras were selected for crystallization and X-ray crystallography experiments to allow determination of their structural characteristics.

2. Random systematic mutagenesis to generate HRV14:HIV-1 gp41 ELDKWA Libraries

The ELDKWA (SEQ. ID. NO.: 1) epitope was introduced into the HRV14 neutralizing immunogenic site II (NIm-II) of viral protein 2 (VP2) of HRV14 (FIG. 5A-B). The core ELDKWA sequence was flanked by one, two, four, or six biased (B) amino acids. The biased linker residues were designed to each have a 50% chance of being any of the 20 amino acids (X) and a 50% chance of being one of the most prevalent HIV amino acids identified at the corresponding site in the HIV-1 ELDKWA sequence (SEQ. ID. NO.: 1)(based on sequences from more than 600 HIV-1 isolates belonging to different clades, as represented in the Los Alamos Database (Korber et al., 1999; FIG. 1, FIG. 5C). By the same reasoning, the first residue of ELDKWA (SEQ. ID. NO.:1) was encoded as E (Glu) 60% of the time and A (Ala) 40% of the time, with the LDKWA (SEQ. ID. NO.:3) sequence held constant to reflect the highly conserved nature of this sequence (although the latter sequence is not 100% conserved among HIV-1 isolates). The (E/A)LDKWA and adjacent biased sequences were then flanked by C (Cys) residues to favor the formation of a disulfide bond, as it is known that disulfide bonds confer higher stability to proteins, and at the same time, might lead to a better presentation of the epitope. The Cys residues were, in turn, flanked by one randomized linker residue (X). The entire cassette was inserted between residues Ser 158 and Val 162 of the VP2 protein of HRV14. With this design, 16 oligonucleotide pairings were performed with each of four N-terminal encoded oligonucleotides with each of four C-terminal oligonucleotides (for which the number of biased linker residues encoded were 1, 2, 4, or 6). Pairs of N-terminal and C-terminal oligonucleotides were hybridized and extended with Klenow DNA polymerase I in order to obtain double-stranded ELDKWA-encoding cassettes. They were subsequently digested with Apa I and Cla I and ligated to the unique complementary restriction sites on the p3IIST plasmid that contains the cDNA of HRV14. Within the restriction sites Apa I and Cla I of the p3IIST plasmid (FIG. 5A), a nonsense cassette was encoded, so non-recombinant plasmid could not be translated into live virus, thus eliminating the possibility of contamination with wild-type virus. The 16 plasmid pools were given their names after the number of B linker residues encoded at both N— and C-terminal sides of the ELDKWA epitope (i.e., pool 41 contains cassettes that were created by hybridizing an N-terminal oligonucleotide encoding four biased linker residues with a C-terminal oligonucleotide encoding one biased linker residue).

E. coli DH10B cells were transformed by electroporation with the mutagenic plasmids from the 16 sets of ELDKWA libraries that encoded from 1.6×10⁵ to 1.6×10¹⁸ possible plasmid members per library. 1.2×10⁵ to 3×10⁷ bacterial transformants were obtained per library. To produce infectious RNAs, the extracted plasmid DNA was linearized at a site downstream of the polyA tract, and in vitro transcription was performed using T7 RNA polymerase. The resulting RNAs were transfected into H1-HeLa cells, yielding 1.9×10⁴ to 1.1×10⁶ chimeric viruses per library (totalling 2.8×10⁶ library members) that underwent partial purification and immunoselection.

### Example 2

### Immunoselection of HRV14:HIV-1 GP41 ELDKWA Libraries

The most antigenic pools were subjected to further immunoselection using different immunoselection approaches.

The immunoselection conditions were varied to enrich for chimeric viruses with the ELDKWA epitope transplanted in an immunologically relevant conformation. In previous work performed with chimeric rhinovirus libraries (Resnick et al., 1995; Smith et al., 1998), a direct immunoselection approach with one or more antibodies was used. In this case alternative approaches were used in order to yield pools richer in immunogenic chimeras. A preliminary screening, measuring the ability of the immunoselected virus pools and their unselected parent pools to bind mAb 2F5, was performed in order to monitor the evolution of the antigenic characteristics of the pools after each immunoselection round (using direct ELISAs).

Competitive Immunoselection.

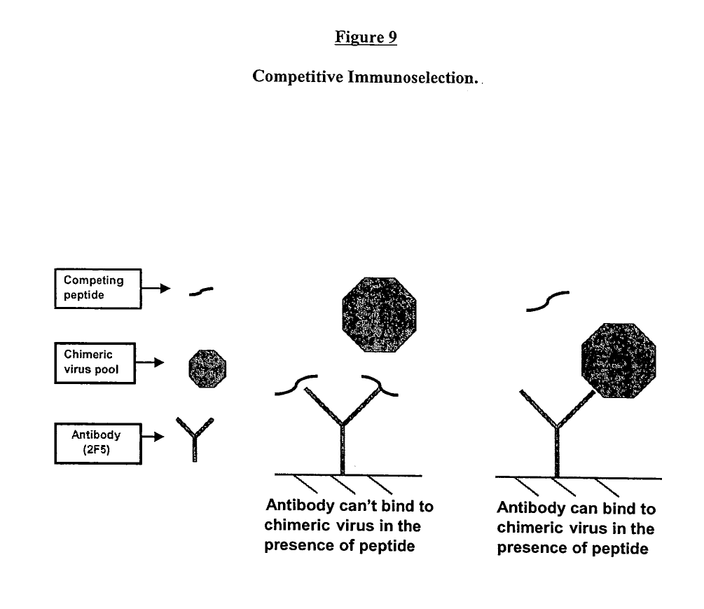

For pool I, which consisted of members of libraries 12, 14, 16, 42, 44, 46, and 64 (FIG. 7), a competitive immunoselection approach was used. The assay is based on the ability of the chimeric viruses to bind to immobilized 2F5 in the presence of a competing peptide with the sequence LELDKWASL (which was shown previously to bind with high affinity to 2F5; Tian et al., 2002). It was expected that the presence of the competing peptide would lead to more stringent conditions for the viruses to bind 2F5, providing higher selectivity. It was expected that only chimeras displaying ELDKWA in a conformation that looked more similar to the native epitope would be able to compete against the LELDKWASL peptide for mAb 2F5 binding (FIG. 9). A broad concentration range of competing peptides was used in order to enrich for more antigenic virus pools. (See FIG. 9)

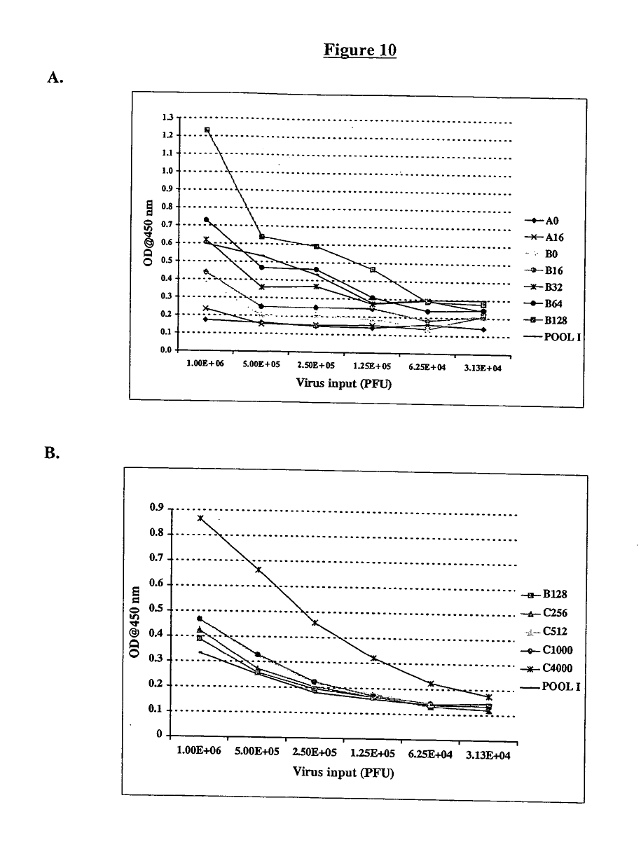

Subsequent rounds of increasingly stringent immunoselection (lowering the amount of mAb 2F5, and/or increasing the amount of the competing peptide) were performed (FIG. 7) and samples of the viruses generated after each immunoselection round were analyzed for their antigenicity (based on the ability to be recognized by mAb 2F5) in a direct ELISA. FIG. 10 illustrates that some of the immunoselected pools generated from Pool I were enriched for antigenic members, as their ability to bind 2F5 increased at each round in which the stringency conditions were raised. FIG. 10A shows that. Pools from early (less stringent) immunoselection rounds with Pool I, such as A0 and B0 which were selected with 0.1 and 0.05 μg/ml of 2F5 and no peptide competition, while pools immunoselected with low concentrations of competing peptide, such as A16, B16, B32, and B64 (which were selected with 16, 16, 32, and 64 pmol/well, respectively) were not better at binding 2F5 than the unselected parent pool. However, pools that were immunoselected with 64 or more pmol/well of competing peptide (B128, etc.) showed an increase in the ability to bind mAb 2F5 compared to the unselected parent pool. Moreover, this 2F5-binding ability increased in parallel with increases in the competing peptide concentration for each immunoselection round (in which 2F5 concentrations were decreased and peptide concentrations increased relatively to each previous round; FIGS. 10A and 10B).

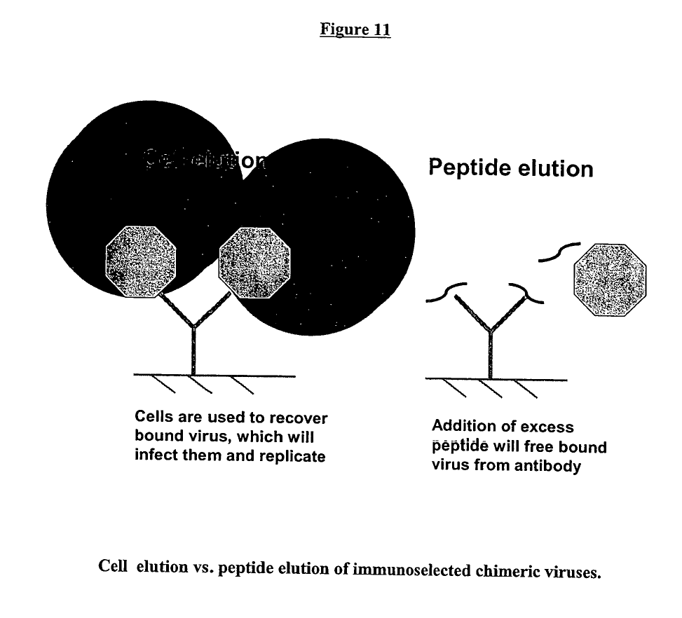

Pool II, consisting of members of libraries 14 and 44, was subjected to one round of competitive immunoselection, and then was subjected to either a second round of competitive immunoselection, or a round of immunoselection with no peptide competition but with elution of bound virus by peptide. Elution with LELDKWASL peptide contrasts with the approach used in previous immunoselection experiments in which elution of the antibody-bound virus was done with the addition of H1-HeLa cells. The viruses propagated in cells can be recovered from the cells. With this type of elution there is a concern about losing the viruses that are bound to antibody so tightly that they might not be able to infect and propagate in the cells used for elution, or they might propagate at a slower rate, which could result in the loss of some of the most valuable (antigenic) members of the pool. Alternatively, the LELDKWASL (SEQ. ID. NO.:4) peptide was used in high concentrations (75-300 μM) to disrupt the antibody-virus association in order to recover antigenic chimeras that might have otherwise been lost with regular cell elution (FIG. 11). The immunoselected viruses produced from the second round of immunoselection without peptide competition (BN) of Pool II were eluted with the LELDKWASL peptide.

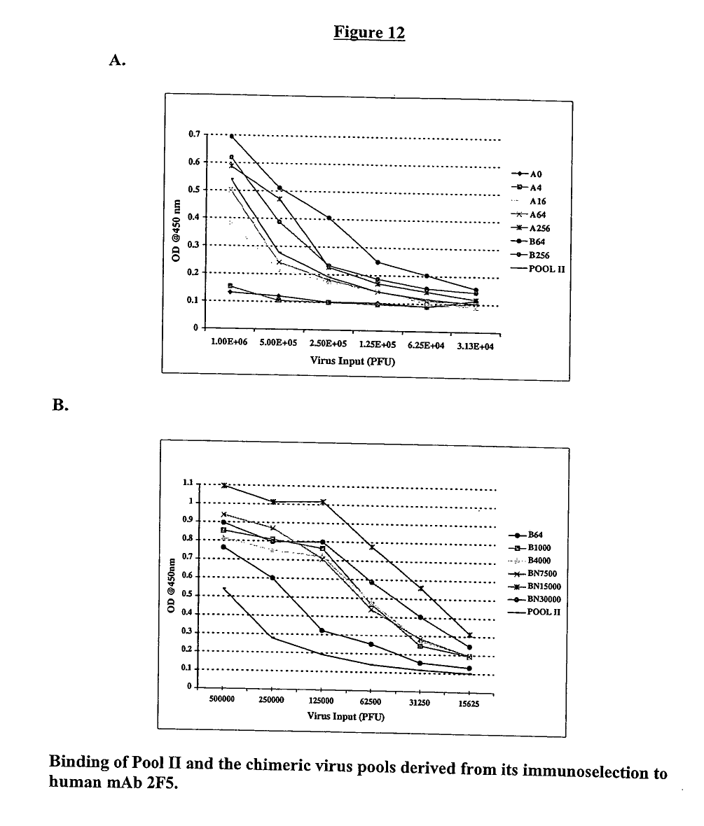

FIG. 12 shows the binding ability of the immunoselected virus pools obtained from this experiment. As with Pool 1, the increasingly stringent conditions enriched for groups of viruses that bound 2F5 better than the unselected pool. The BN pools from peptide elution exhibited a greater ability to bind mAb 2F5 compared to pools eluted with cells (FIG. 12B); the conditions using 1000-4000 pmol/well of competing peptide were also among the best binders to mAb 2F5. Thus, peptide elution effectively rescued chimeras that bound 2F5 tightly and that might have been lost if eluted with cells.

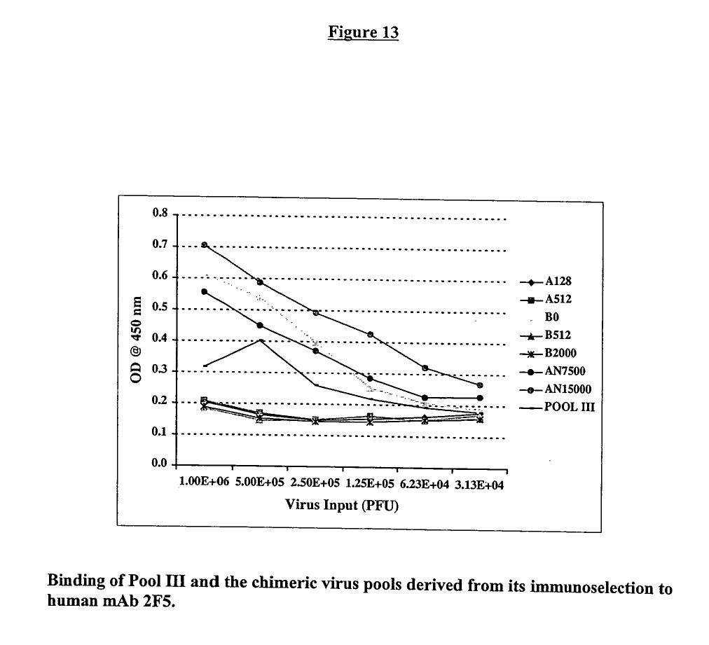

Pool III, consisting of members of libraries 11, 12, 14, 16, 21, 22, 24, 26, 41, 44, 61, 62, and 64, was subjected to either two sequential competitive immunoselection rounds, or to one non-competitive immunoselection round with peptide elution. The pools derived from this pool exhibited a decreased afinity for mAb 2F5 compared to the pools derived from Pools I and II. Apparently, the stringency of the immunoselection conditions led to loss of diversity, and the unselected pool generally exhibited a lower ability to bind mAb 2F5 compared to unselected Pools I and II. The peptide concentrations used for both competition and elution were slightly lower but overlapping some of the ones used for Pools I and II (128-2000 pmol/well of peptide for competition, 7500-15000 μM peptide for elution)in order to identify conditions that were stringent enough to select for the most antigenic chimeras within the group, while preserving diversity. Surprisingly, the pool immunoselected without competition exhibited greater antigenicity. This was particularly surprising given the steady patterns seen for Pools I and II immunoselected with peptide competition. Perhaps because this pool would be expected to be extremely diverse (with respect to insert size of the chimeras), the faster growing viruses (not necessarily the more antigenic) had an advantage. As the AN pools (which had been selected without competition and eluted with peptide, which should have eliminated the advantage for faster growing viruses) were more antigenic than their counterparts exposed to peptide competition (FIG. 13).

Members from the AN pool were analyzed with respect to antigenicity and immunogenicity with that for members of the most antigenic groups generated from Pools I and II.

Immunoselected pools that had better ability to bind 2F5 in the direct ELISA (FIGS. 10, 12, and 13) were identified as the most likely to include the most antigenic chimeras and, therefore, were selected for isolation and characterization of. Immunoselected chimeric virus clones were isolated from immunoselected pools: from Pool I, including B64, B 128, B256, C512, C1000, C4000. Additionally, a subset of library 64, isolated clones from pools B32, C16, C32, and C128, were analyzed. From Pool II, immunoselected pools included B64, B256, B1000, B4000, BN7500, BN15000, and BN30000. The only immunoselected pool chosen from Pool III was AN 15000.

### Example 3

### Sequence Analysis of HRV14:HIV-1 GP41 ELDKWA Chimeric Viruses

Sequence analysis was performed on both unselected and immunoselected viruses, to identify important differences in amino acid residues among the different groups. On average, 10 plaques per selected pool were sequenced (FIG. 5C). It was observed that in pools where the immunoselection conditions had been very stringent, fewer unique clones were isolated, leading to a loss of diversity. An extreme case was seen with Pool I round C, from which one clone, 14-C4000-1, appeared for 10 of the 19 plaques isolated, and the actual number of unique clones identified within this particular pool was 3. Other clones from immunoselection round C using less stringent conditions were also repeated, but to a lesser degree, appearing in 2 or 3 out of 10 or 15 of the plaques isolated. After sequencing immunoselected clones from Pool I, it was decided to adjust the stringency conditions and the number of immunoselection rounds for the other pools, with the expectation that while still enriching for potentially immunogenic clones, a larger diversity would be observed. As a result, plaques isolated from immunoselected Pools II and III, were found to have few or no sequence repetitions at all.

A total of 69 sequences from unselected chimeras, and 31 from immunoselected were obtained (FIG. 5C). The library with the largest overall diversity was 14, followed by 44, and then 64. Even though there were differences in the overall expected diversity and the transfection efficiencies among the libraries, the larger representation of sequences from libraries containing a C-terminal linker with either one or four biased residues is likely to be due to the fact that the corresponding oligonucleotide encoding these linkers were contaminating the other end terminal linker oligonucleotides.

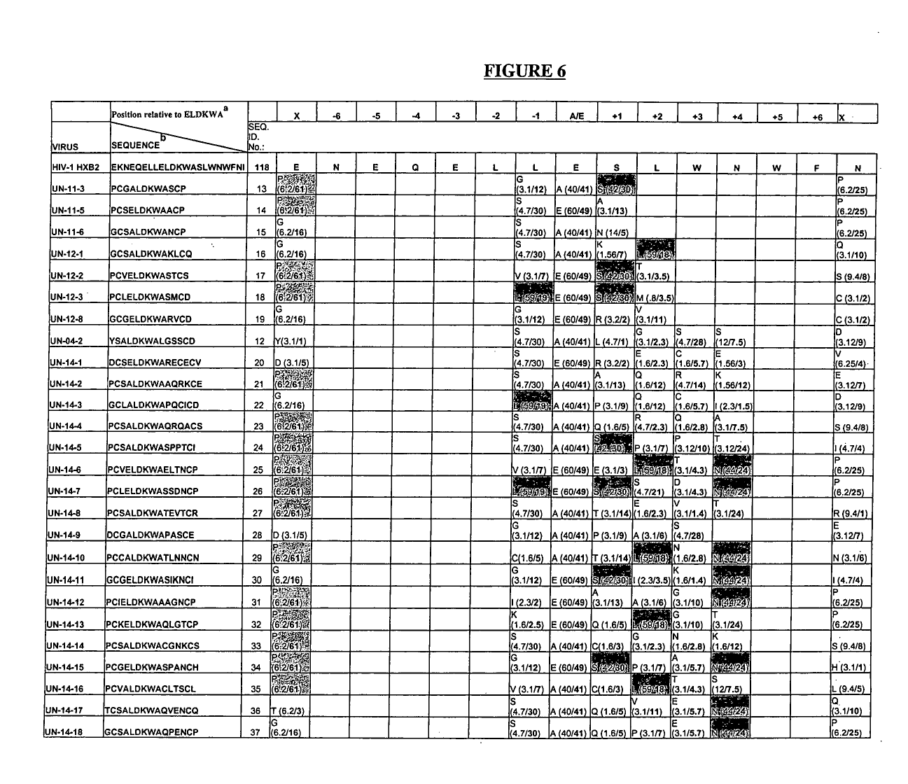

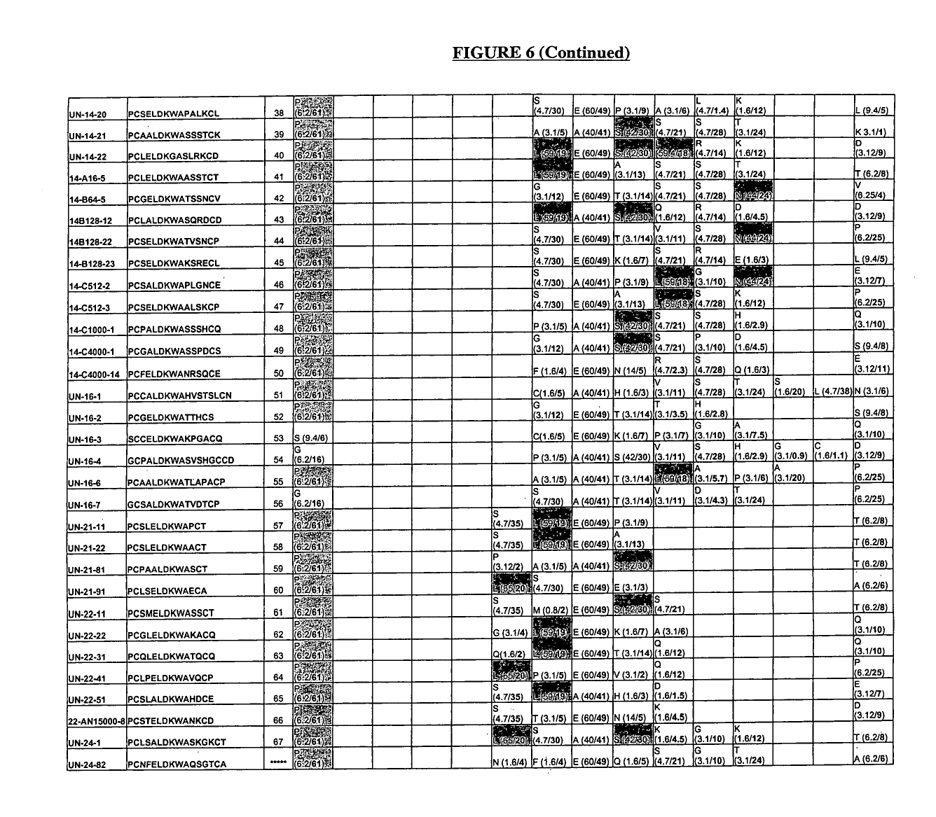

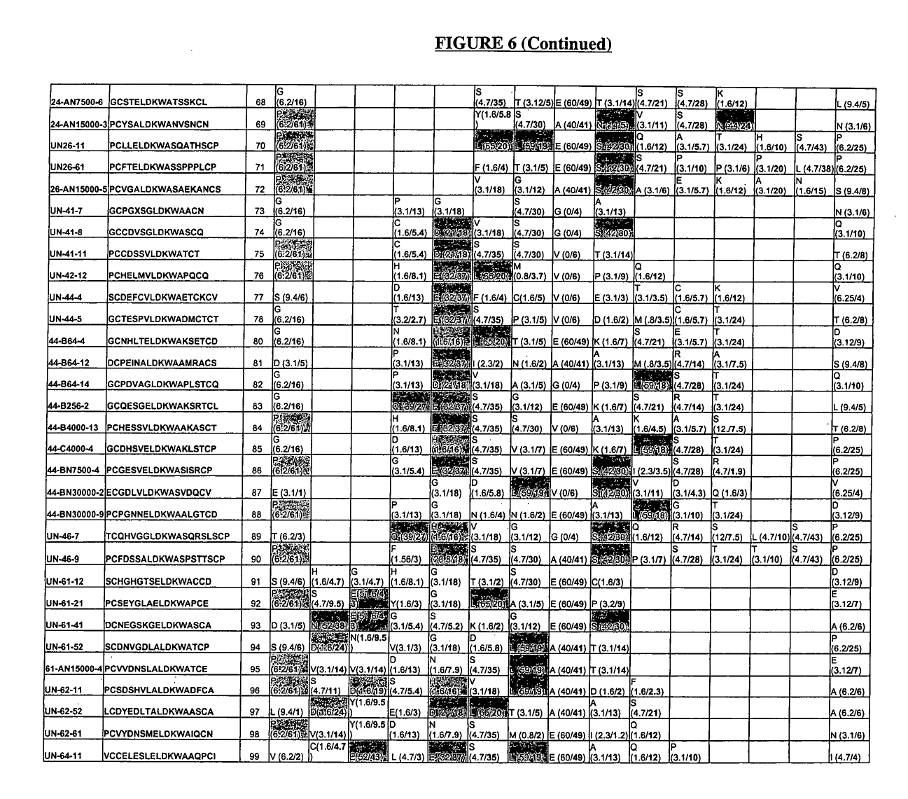

A remarkable and surprising result was observed among all the chimeras sequences is that the N-terminal residue, which was encoded to be fully randomized (FIG. 6), turned out to be Pro in 61% (6.2% expected) of the sequenced chimeras (taking into account both unselected and immunoselected clones; FIG. 6). This residue may have significant influence on the structural requirements for accommodation of the insert into the capsid of HRV14 in such a way as to make the virus viable. Pro is a turn-forming residue; it might be needed to create a turn when switching from the HRV native sequence to the inserted foreign sequence. Furthermore, the second most prevalent residue at this position (represented 16% of the time) is Gly, another turn-forming residue. Accordingly, 73% of the sequenced chimeras incorporated Pro or Gly, turn-forming residues. On the C-terminal randomized linker there was also a non-random prevalence of Pro (25% of the C-terminal residues compared with an expected 6.2% of the residues, and encoded by the oligonucleotides); its presence may influence virus viability. This is supported by the observation that from eight recombinant plasmids sequenced, only one (12.5% of a maximum of 7 plasmids that could encode viable viruses) encoded a Pro as the N-terminal linker residue. The sequences of the inserts corresponding to the plasmids are shown in Table I.

TABLE I

Sequences encoded by recombinant plasmids
containing ELDKWA inserts.

| | | |
|---|---|---|
| Insert size | | |
| (# residues | | |
| encoded) | Sequence encoded^{a} | SEQ. ID. No.: |

| | | |
|---|---|---|
| 13 | RCFALDKWATSCS | (SEQ.ID.NO.:5) |
| 12 | SCLALDKWAL*CI | (SEQ.ID.NO.:6) |
| 13 | PCAELDKWAXTXN | (SEQ.ID.NO.:7) |
| 13 | CCSALDKWAIXCK | (SEQ.ID.NO.:8) |
| 15 | XCVELDKWASXCX | (SEQ.ID.NO.:9) |
| 15 | XCGELDKWAXSCXXG | (SEQ.ID.NO.:10) |
| 12 | CVELDKWASTCX | (SEQ.ID.NO.:112) |
| 17 | SGDEFCVLDKWAETCKC | (SEQ.ID.NO.:113) |

^{a}An asterisk (*) denotes the presence of a stop codon at that position. X indicates that the sequencing could not identify the residue encoded at that position.

Most biased residues were not present in the percentages designed; however, they reflected an increased in representation when they had been biased by the design, as it is the case of Leu at positions −1 and −2 N-terminal to ELDKWA, and +2 C-terminal to ELDKWA. These where designed to occur at frequencies of 59, 65, and 59% respectively, but were only present in 19, 20, and 18% of the chimeras sequenced. Asn, Glu, Gln, and Asp at positions −6, −5, −4, and −3, respectively, N-terminal to ELDKWA were represented at percentages slightly closer to the expected (52, 52, 39 and 21% expected vs. 38, 43, 27 and 18% observed). The same situation occurred at the C-terminal positions +2 and +4 corresponding to residues Ser and Asn (42 and 44% expected vs. 30 and 24% observed). Glu at position −3 N-terminal to ELDKWA actually was represented at a slightly higher percentage (37%) than expected (32%). Almost all residues biased by design appeared in the expected positions, with exceptions in the third and fifth positions C-terminal to ELDKWA, where Trp should have been encoded 50% of the time. Instead, Trp was never observed (for either selected or unselected chimeras; FIG. 6). Furthermore, no Trp at all were observed at any of the linker positions flanking the ELDKWA epitope, which implies that even though Trp can be presented by the chimera in the middle of the insert (within the ELDKWA sequence), it is present at a second site or is closer to the native VP2, which might make the virus inviable.

It is also important to recognize that naturally occurring HIV-1 residues flanking the ELDKWA epitope were found for most of the chimeras, even when they were not biased. The residue, Ser was identified in percentages higher than expected at various positions. At the first and second (N-terminal) positions preceding ELDKWA, Ser was present in percentages of 30% and 35%, respectively, compared to 4.7% expected by design. Ser also appeared at a higher percentage (28%) than expected (4.7%) at the third position C-terminal ELDKWA. Thr also appeared with a 24% frequency when the expected one was 3.1% at the fourth position N-terminal to ELDKWA. Glu and Ala (at the first position of (E/A)LDKWA) appeared with a 49 and 40% frequency, respectively. The original design encoded these residues in a 60/40 ratio, however it seems that a single nucleotide change could have mutated either the codon GCA or GAA (that encode Ala and Glu respectively) to GGA or GUA that encode Gly and Val, respectively, which were the residues that without being in the design appeared at this position with frequencies of 4% and 6% respectively.

The proportions at which certain residues are represented within the subset of sequenced chimeras may reflect the result of the initial design, and the selection of the best combinations that led to the production of viable viruses. However, it has to be taken into account that the sequenced chimeras do not equally represent all of the 16 possible insert sizes and this analysis was only based on the entire set of sequences, not on each subset of viruses containing different insert sizes.

The sequence features observed in the chimeras isolated do not faithfully represent the design at positions where some residues were expected to be biased or fully randomized. But it is also important to remember that some of the sequence combinations expected might have not yielded viable viruses; what is observed here is the product of the original design and the selection for combinations of linker residues that did not dramatically alter the rhinovirus capsid and allowed for the production of viable chimeras.

The group of 31 immunoselected clones was analyzed separately to see if any difference in the prevalence of biased residues or naturally occurring HIV-1 residues was observed that could give an idea if immunoselection had a predilection for a particular set of residues at certain positions apart from ELDKWA. The proportions at which biased and non-biased residues were represented in this set were close to the ones observed in all the sequenced chimeras presented in FIG. 6.

One of the immunoselected chimeras, 14-C4000-1 was analyzed for the presence of a disulfide bond, the digestion with trypsin under non-reducing conditions showed that there was no disulfide bond present in this particular isolate.

### Example 4

### Antigenic Characteristics of Immunoselected HRV14:HIV-1 GP41 ELDKWA Chimeric Viruses

Twenty seven of 31 immunoselected and sequenced chimeric virus clones were further analyzed with respect to their antigenic characteristics. Some of the immunoselected pools from which individual chimeric virus clones were isolated contained clones found in other pools. From Pool II, immunoselected pools B I000, and BN15000 contained some members that had already been identified in the pools B4000, and BN30000, respectively.

Antigenicity was analyzed by two assays: a direct ELISA and a microtiter 2F5 neutralization assay, which were focused on the recognition and neutralization of the chimeras by mAb 2F5. The approach postulated that chimeras that are strongly recognized by this potently and broadly neutralizing antibody display conformations of the ELDKWA epitope in ways that mimic the natural epitope and enable them to elicit the production of similar antibodies. A direct ELISA was performed on immunoselected clones and their parent pools in order to compare if the clones had at least the same mAb 2F5 binding ability as the relevant parent pool. Additionally, to obtain information on the antigenicity of the chimeric viruses, a mAb 2F5 microtiter neutralization assay was performed. This assay has been shown to be useful in identifying some viruses with better probabilities of being promising immunogens (as they have shown an ability to elicit the production of anti-HIV neutralizing antibodies; (Smith et al., 1998; Bayman et al., in preparation) based on the amount of antibody needed for the neutralization of the chimeric viruses.

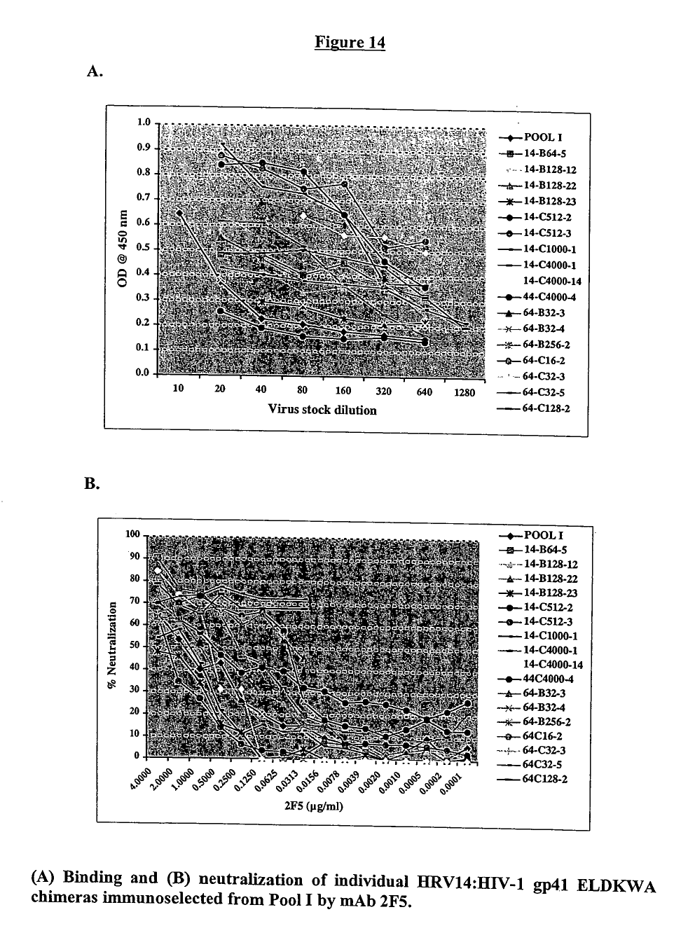

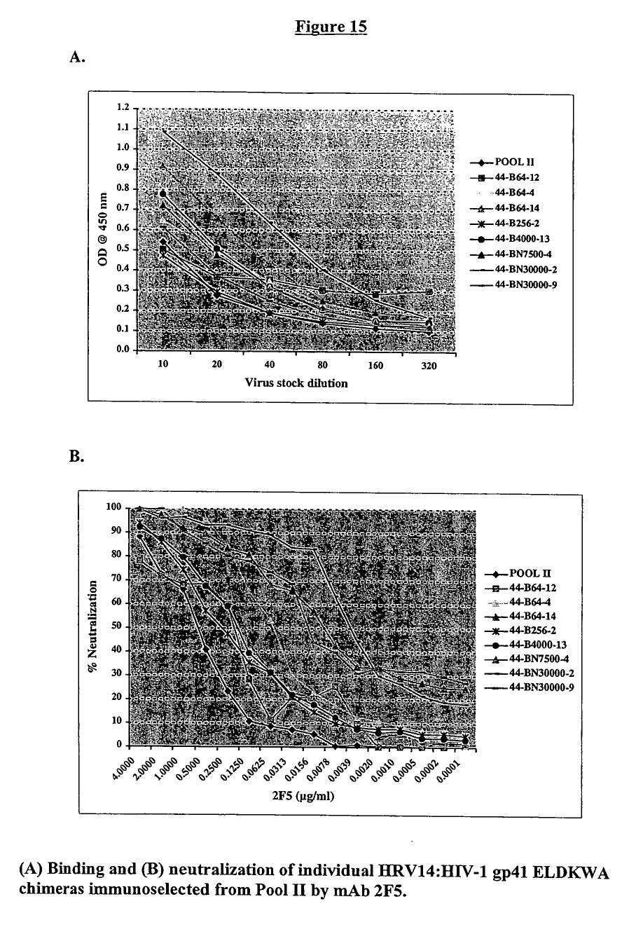

FIGS. 14-16 show side-by-side results of the ELISA (2F5-binding ability) and microtiter 2F5 neutralization assay (sensitivity to neutralization by 2F5) results for Pools 1, II, and III and the individual clones derived from them. For Pool I (FIG. 14A), from which the largest number of immunoselected clones was analyzed (17 chimeras). The majority of the 17 immunoselected viruses had an enhanced ability to bind to mAb 2F5 as compared to the parent pool. Chimeras 14-B128-22, 14-C512-3, 14-C1000-1, 14-C4000-14, and 44-C4000-4 stood out for being able to bind 2F5 and yield a high signal at extremely high dilutions. These observations were confirmed by calculations of the reciprocal dilutions of the virus titer (from an initial stock of 1×10⁸ PFU/ml) corresponding to an OD₄₅₀ of 0.5 upon binding to immobilized 2F5 (Table II). The neutralization assay for clones selected from Pool I (FIG. 14B) showed similar results, though in a slightly less obvious trend. Some of the clones, most notably 14-C512-2,64-B32-3, 64-C16-2, 64-C32-3, 64-C32-5, and 64-C128-2, were more sensitive to neutralization by 2F5 than the unselected parent pool. This is reflected in Table II, where the inhibitory concentration of 2F5 that led to 50% neutralization of the chimeras is shown.

TABLE II
MAb 2F5 binding and neutralization of immunoselected chimeric
HRV14:HIV-1 gp41 ELDKWA viruses and the unselected pools
from which they were derived.

| | | 2F5 IC₅₀^{b} |
|---|---|---|
| Chimeric Virus | ELISA Titer^{a} | (μg/ml) |
| POOL I^{c} | 16 | 1.45 |
| 14-B64-5 | 37 | 0.29 |
| 14-B128-12 | 22 | 0.49 |
| 14-B128-22 | 470 | 0.57 |
| 14-B128-23 | 5.3 | 5.90 |
| 14-C512-2 | 7.0 | 0.05 |
| 14-C512-3 | 460 | 3.00 |
| 14-C1000-1 | 220 | 1.50 |
| 14-C4000-1 | 86 | 0.98 |
| 14-C4000-14 | 650 | 0.64 |
| 44-C4000-4 | 270 | 0.66 |
| 64-B32-3 | 29 | 0.05 |
| 64-B32-4 | 9 | 0.49 |
| 64-B256-2 | 23 | 0.23 |
| 64-C16-2 | 20 | 0.07 |
| 64-C32-3 | 54 | 0.05 |
| 64-C32-5 | 14 | ND^{d} |
| 64-C128-2 | 82 | ND |
| POOL II^{e} | 13 | 0.68 |
| 44-B64-4 | 19 | 0.06 |
| 44-B64-12 | 10 | 0.21 |
| 44-B64-14 | 31 | 0.16 |
| 44-B256-2 | 9.3 | 0.31 |
| 44-B4000-13 | 20 | 0.24 |
| 44-BN7500-4 | 18 | 0.01 |
| 44-BN30000-2 | 57 | 0.01 |
| 44-BN30000-9 | 15 | 0.02 |
| POOL III^{f} | 3.2 | 3.90 |
| 22-AN15000-8 | 3.1 | 8.20 |
| 24-AN15000-3 | 5.8 | 1.50 |

^{a}ELISA titer, the reciprocal dilution of the chimeric viral pool/clone (from an initial stock of 1 × 10⁸ PFU/ml) corresponding to an OD₄₅₀ of 0.5 upon binding to immobilized mAb 2F5
^{b}IC₅₀, the inhibitory concentration of mAb 2F5 that led to 50% neutralization of chimeric virus
^{c}Consisting of members of libraries: 12, 14, 16, 42, 44, 46, and 64
^{d}Not determined. The antibody dilutions at which these chimeras were tested were not great enough to calculate the corresponding 2F5 IC₅₀s.
^{e}Consisting of members of libraries: 14 and 44
^{f}Consisting of members of libraries: 11, 12, 14, 16, 21, 22, 24, 26, 41, 44, 61, 62, and 64

In the case of Pool II (FIG. 15A), all but one clone, 44-B256-2 had an increased ability to bind 2F5 compared to that of the parent unselected pool. Among the clones that had the greatest 2F5-binding ability within this pool were those from the pools eluted with peptide (44-BN30000-2, 44-BN30000-9, and 44-BN7500-4), and clones 44-B4000-13, 44-B64-4, and 44-B64-14 isolated by competitive immunoselection. In the cases of the neutralization assay (FIG. 15C), all clones immunoselected from this pool were more sensitive to neutralization by 2F5 than the parent pool, with clones 44-BN30000-2, 44-BN7500-4, and 44-BN30000-9, being the most outstanding in their sensitivity to neutralization by 2F5.

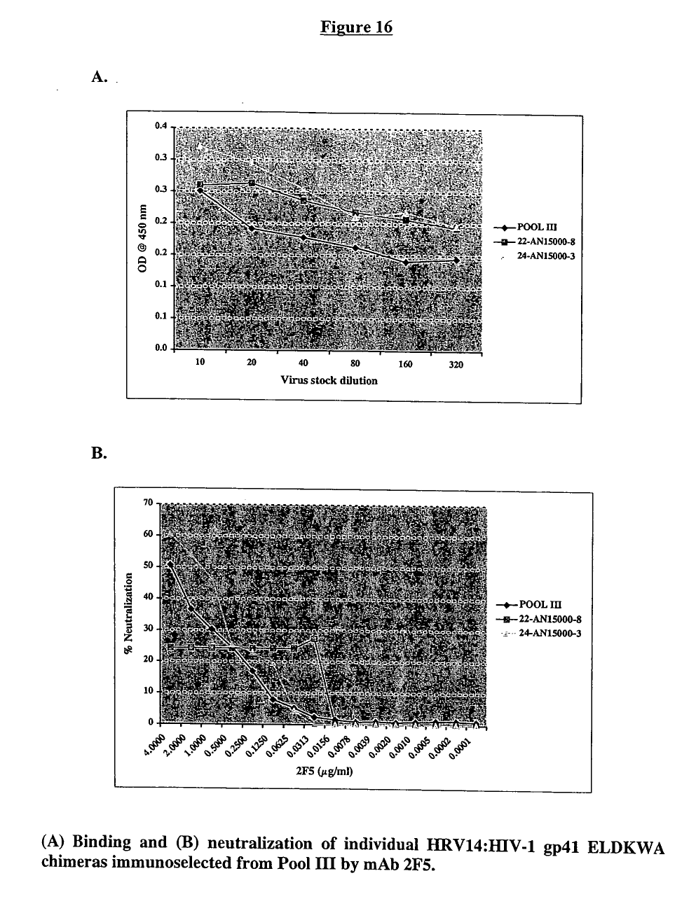

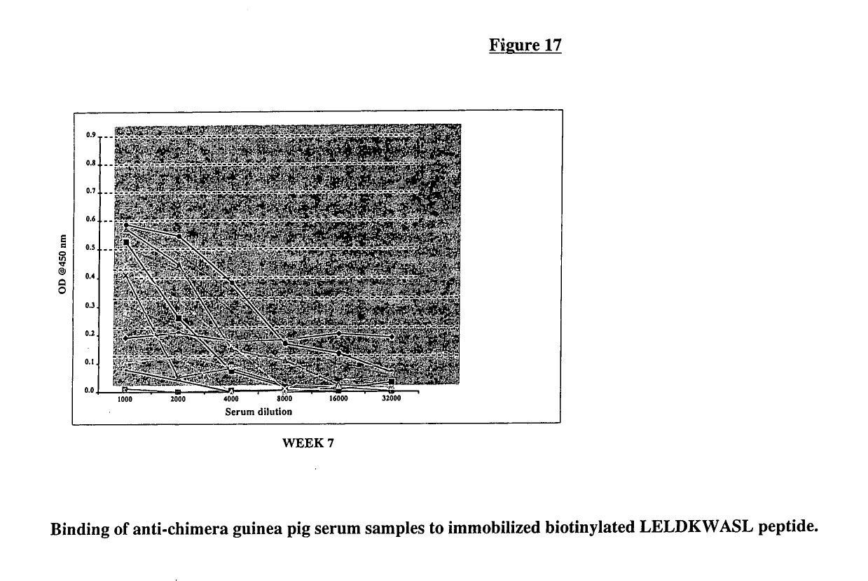

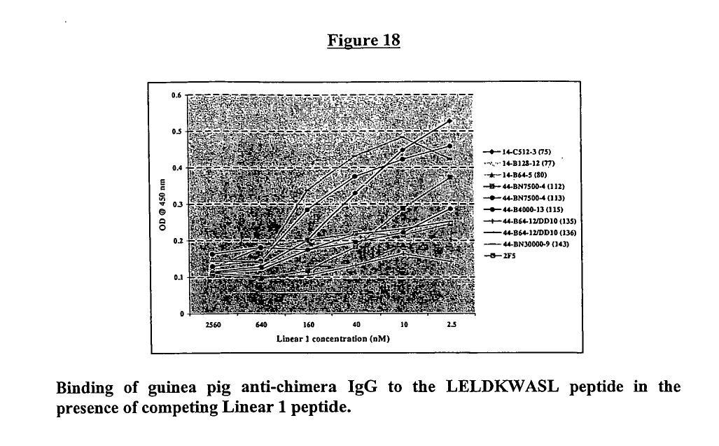

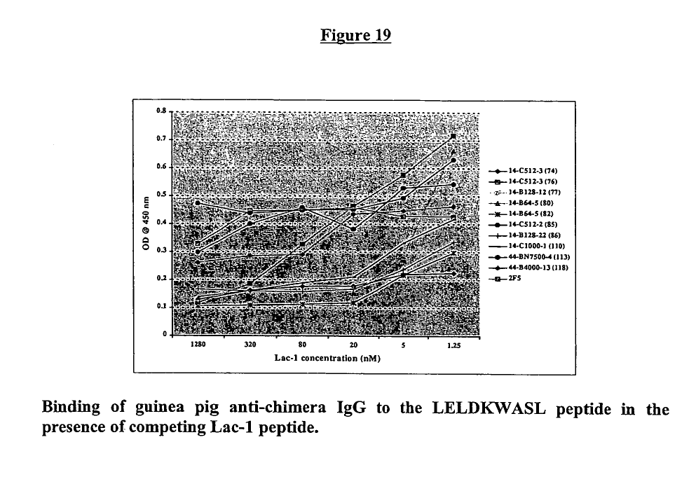

Two immunoselected clones from Pool III were analyzed for their antigenicity:viruses isolated from the AN15000 and AN30000 pools which exhibited improved antigenicity compared to the parent unselected pool (FIG. 13). FIG. 16 shows the binding (ELISA) and neutralization sensitivity by 2F5 of these two clones compared to their parent unselected pool. Chimeric virus 24-AN15000-3 showed slight improvement in binding ability to 2F5 (and neutralization sensitivity) than the parent unselected pool. Chimeric virus, 22-AN15000-8 showed improved 2F5 binding compared to the parent pool at all but the most concentrated virus dilutions.

The antigenicity assays indicated that 89% (24 of 27) of the clones isolated after immunoselection had better antigenic properties (as measured by ELISA and/or neutralization assay) than their unselected parent pools. Thus, immunoselection enriched for antigenic chimeras, and peptide elution had been able to rescue some of the most antigenic chimeras.

The ELISA and microtiter neutralization assay data (2F5 IC₅₀) values for each chimera and for the unselected parent pool were compared (Table II). The ELISA titer was determined by calculating (by interpolation) the virus dilution at which an OD₄₅₀ of 0.5 was observed. Therefore the viruses presenting the higher ELISA titers would be considered the most antigenic (with respect to this assay). The 2F5 IC₅₀s were designated as the concentration of mAb 2F5 (μg/ml) needed to achieve 50% inhibition of virus growth (neutralization). These were also calculated by interpolation of the data obtained in the microtiter neutralization assay. The viruses neutralized by the lowest concentrations of mAb 2F5 were classified as the most antigenic. Most (92%; 25 out of 27 chimeras) of the antigenicity assay results indicated increased chimera antigenicity with respect to their parent pool. Overall, the clones recovered by peptide elution from Pool II: 44-BN7500-4, 44-BN30000-2 and 44-BN30000-9 were the most sensitive to neutralization by 2F5.

Some members of pool I (14-B128-22, 14-C512-3, 14-C1000-1, 14-C4000-14, and 44-C4000-4) were the ones that had the highest increase in their ELISA titers compared to the clones isolated from the other pools.

These chimeras, with either their increased ability to bind 2F5, or increased sensitivity to mAb F25 neutralization were assessed for immunogenic characteristics.

### Example 5

### Immunogenic Characteristics of HRV14:HIV-1 GP41 ELDKWA Chimeric Viruses

From the 27 unique immunoselected clones that were analyzed for their antigenicity, 20 were selected for use in guinea pig immunizations. Clones 14-B128-23, and 22-AN15000-8 were not selected due to low antigenicity (according to their ELISA and 2F5 neutralization titers).

Three guinea pigs were immunized with each of the selected chimeras. The chimeras used for immunization were partially purified and quantified by PFU. It was determined that approximately 1×10⁹ PFU corresponded to 10 μg of purified virus. The average amount of virus used per immunization was 3×10⁹ PFU, equivalent to approximately 30 μg/guinea pig.

Additionally, guinea pigs immunized with chimeras 14-C4000-1, 44-B64-12, 44-B4000-13 and 44-C4000-4 were boosted with 40-80 μg KLH-conjugated ELDKWA-based peptides in order to see if the boost would have an additive effect on the immune response of the animals. The two peptides used were (1) a 9-mer containing the sequence LELDKWASL shown to tightly bind 2F5 (Tian et al., 2002) and (2) a 14-mer containing the sequence EQELLELDKWASLW (SEQ. ID. NO.: 114), shown to be the part of the peptide that remains bound to 2F5 when subjected to proteolysis (Parker et al., 2001).

Two sets of guinea pigs received immunizations with cocktails of chimeras mixed in equal ratios in order to explore the possibilities of eliciting synergistic immune responses among them. A chimeric rhinovirus containing a V3 loop-like epitope with the sequence SVHLGPGRAFYA (SEQ. ID. NO.: 115), DD-10, was used because it has elicited neutralizing antibodies against some HIV-1 primary isolates (Bayman et al., in preparation). One of the combinations was 44-B64-12/DD-10 and the other was 44-C4000-4/44-B256-2/DD10.

1. Ability of the Sera Elicited by HRV14:HIV-1 gp41 ELDKWA Chimeric Viruses to Bind ELDKWA-Based Peptides

The sera elicited by the chimeric virus immunizations were tested for their ability to bind an immobilized biotinylated LELDKWASL peptide in a direct ELISA. The first serum samples received were monitored for their increase in LELDKWASL peptide binding at weeks 0 (prebleed), 7 and 12 (as well as 16 when the samples came from an animal boosted with peptide at week 13; FIG. 17). The general pattern observed was that the sera drawn at week 12 were more reactive to LELDKWASL peptide than the sera drawn at week 7. By the time the second serum sample was obtained the animals would have received three immunizations (as opposed to only two by week 7). In only one case reactivity dropped at week 12, compared to week 7 (serum 70; FIG. 17). Most of the sera analyzed showed reactivity to peptide; the reactivity could vary in level among animals immunized with the same virus (animal to animal variation). However, there was only one case in which an immune serum did not exhibit reactivity to peptide (serum 62; FIG. 17), even though the sera corresponding to the other two animals immunized with the same virus showed moderate to high responses to the immunogen.

Sera was obtained from immunized animals at the terminal bleeds (week 12, or week 12 and 16 for animals receiving a peptide boost). The immunized animals were fully exanguinated at week 12 (or 16) in order to maximize serum collection.

The peptide-binding ability of the guinea pig sera gave a positive or negative indication of peptide reactivity as well as indication of the reactivity level when serum were compared with one another and with control sera (serum from prebleeds or from animals immunized with wild-type HRV 14).

In order to find out if there were differences in the amount of antibody produced by the immunized animals, and to perform quantitative peptide-binding assays, it was necessary to work with known antibody concentrations. All the sera were tested for direct binding to biotinylated LELDKWASL peptide, and the ones with the best peptide reactivity and serum availability were selected for purification of immunoglobulin G (IgG). The IgG fraction of each of the serum samples was isolated by affinity chromatography using protein A column purification. It was found that, on average, the sera contained from 3.5-5 mg/ml of total IgG.

Direct ELISAs were performed to compare the immune sera IgG fractions with 2F5 for their abilities to bind to biotinylated LELDKWASL peptide. Table III summarizes the results of this assay for the sera tested. Notably, the IgG fraction from sera 136 and 100 were able to bind the LELDKWASL peptide approximately 46% as well as the equivalent concentration of 2F5 IgG. This could mean that almost half of the IgG present on these samples was reactive to the LELDKWASL sequence. From the 40 purified IgG samples tested, 24 had a LELDKWASL peptide reactivity of at least 10% that of an equivalent amount of 2F5. Although it is known that HRV14 is a strong immunogen, and expected that the immunoselected chimeras would elicit strong immune responses in animals, these results are outstanding, as the IgG isolated is a polyclonal mixture which can present reactivity to other epitopes on the chimeras (e.g., HRV14 epitopes).

TABLE III
Direct and competitive peptide binding assays performed with
anti-HRV14:HIV-1 ELDKWA chimera IgG.

| | Serum | LELDKWASL |
|---|---|---|
| Immunogen(s) | code^{a} | binding^{b} |
| 14-B64-5 | 80 | 240 |
| 14-B64-5 | 82 | 140 |
| 14-B128-12 | 77 | 150 |
| 14-B128-22 | 86 | 380 |
| 14-B128-22 | 87 | 770 |
| 14-B128-22 | 88 | 290 |
| 14-C512-2 | 85 | 120 |
| 14-C512-3 | 74 | 210 |
| 14-C512-3 | 75 | 350 |
| 14-C512-3 | 76 | 250 |
| 14-C1000-1 | 110 | 280 |
| 14-C4000-1 | 63 | 530 |
| 14-C4000-1 + 14-mer^{d}13 | 63-P | 260 |
| 14-C4000-1 + 14-mer 9 + 13 | 65-P | 660 |
| 14-C4000-1 + 14-mer 9 + 13 | 65-PP | 330 |
| 14-C4000-1 + 14-mer 9 + 13 | 66-P | 140 |
| 14-C4000-1 + 14-mer 9 + 13 | 66-PP | 480 |
| 24-AN15000-3 | 154 | 480 |
| 24-AN15000-3 | 155 | 190 |
| 44-B64-12 + 14-mer 13 | 138-P | 50 |
| 44-B64-12 + 14-mer 13 | 139-P | 30 |
| 44-B256-2 | 147-P | 20 |
| 44-B256-2 | 148-P | 70 |
| 14-C4000-4 + 9-mer 13 wpi | 68-P | 260 |
| 14-C4000-4 | 69 | 530 |
| 14-C4000-4 + 9-mer 13 wpi | 69-P | 260 |
| 44-B4000-13 | 115 | 10 |
| 44-B4000-13 + 14-mer 13 | 118-P | 90 |
| 44-BN7500-4 | 112 | 510 |
| 44-BN7500-4 | 113 | 150 |
| 44-BN30000-2 | 140 | 120 |
| 44-BN30000-9 | 143 | 250 |
| 64-B32-3 | 98 | 600 |
| 64-C32-3 | 100 | 1100 |
| 64-C32-3 | 102 | 1000 |
| 64-C32-5 | 94 | 980 |
| 64-C128-2 | 93 | 890 |
| 44-B64-12/DD-10^{e} | 135 | 120 |
| 44-B64-12/DD-10 | 136 | 1100 |
| 44-C4000-4/44-B256-2/DD- | 144 | 90 |
| HIV-1 | 2F5^{f} | 2400 |

^{a}Serum samples were collected 12 weeks post-inoculation (wpi) unless indicated with a ‘P’ or ‘PP’. In these cases, one (P) peptide or two (PP) peptide boosts were given on weeks(s) indicated and collected at 16 wpi (or 12 wpi following a 9 wpi peptide boost, as for samples 65-P, 66-P, and 67-P)
^{b}Reciprocal dilution of the 1 mg/ml IgG fraction of the serum that resulted in an OD₄₅₀ of 0.5 when bound to LELDKWASL
^{c}IC₅₀, competing peptide concentration yielding a 50% inhibition of serum binding to LELDKWASL alone. Lac-1 = Ac-CNEQELLEKDKWADL-NH₂, a constrained peptide with a lactam bridge connecting the underlined K and D residues; linear-1 = Ac-CENEQELLELDKWASL-NH₂. Blank cell = not tested due to limitation of samples availability
^{d}An immunization boost was given at week 9 and/or 14 containing KLH-conjugated EQELLELDKWASLW peptide (14-mer), or KLH-conjugated LELDKWASL peptide (9-mer)
^{e}DD-10, a chimeric virus containing a V3 loop-like insert, SVHLGPGRAFYA, was immunized simultaneously with the HRV14:HIV-1 ELDKWA chimera(s)
^{f}2F5 IgG was tested for peptide binding ability using the same conditions as for the IgG fractions of the sera samples

Remarkably, some of the sera bound the competing peptides not only with greater affinity than LELDKWASL but also with greater affinity than 2F5 itself. Particular interest was put into IgG samples that strongly bound to Lac-1, as this peptide is well recognized by 2F5, and could represent a possible immunogenic conformation of the epitope (Tian, 2003). Therefore, it could be that antibodies present in some or all of the sera that strongly recognize the Lac-1 peptide would have HIV neutralization ability.

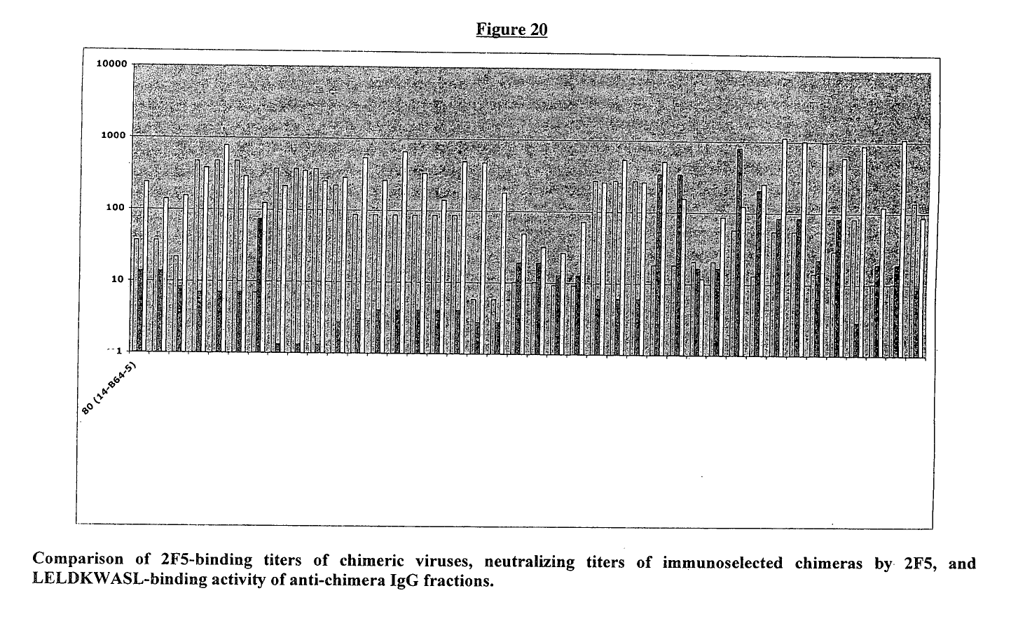

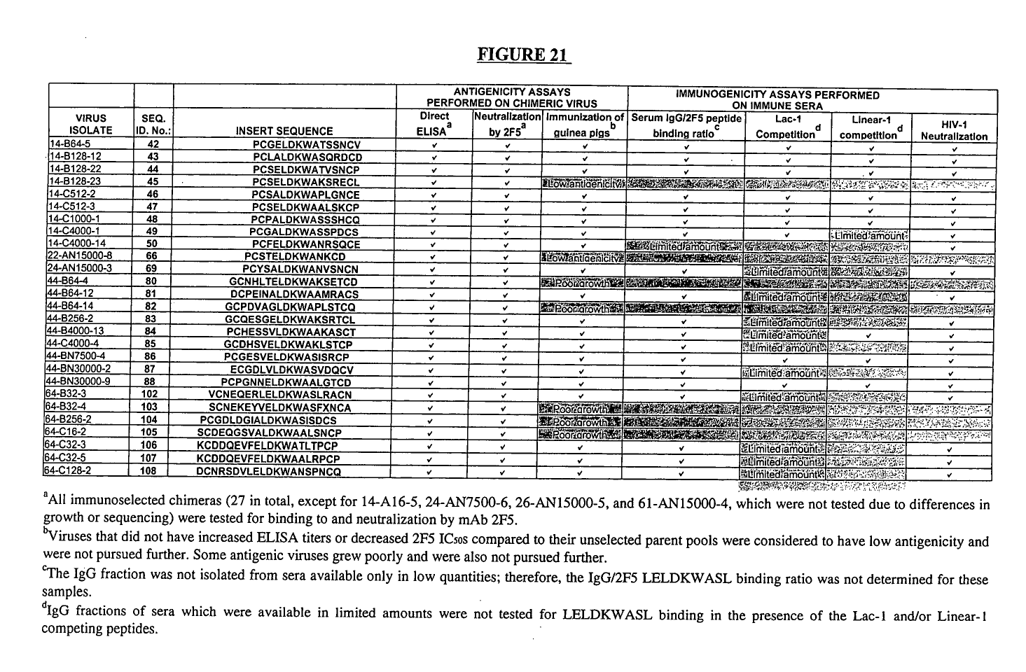

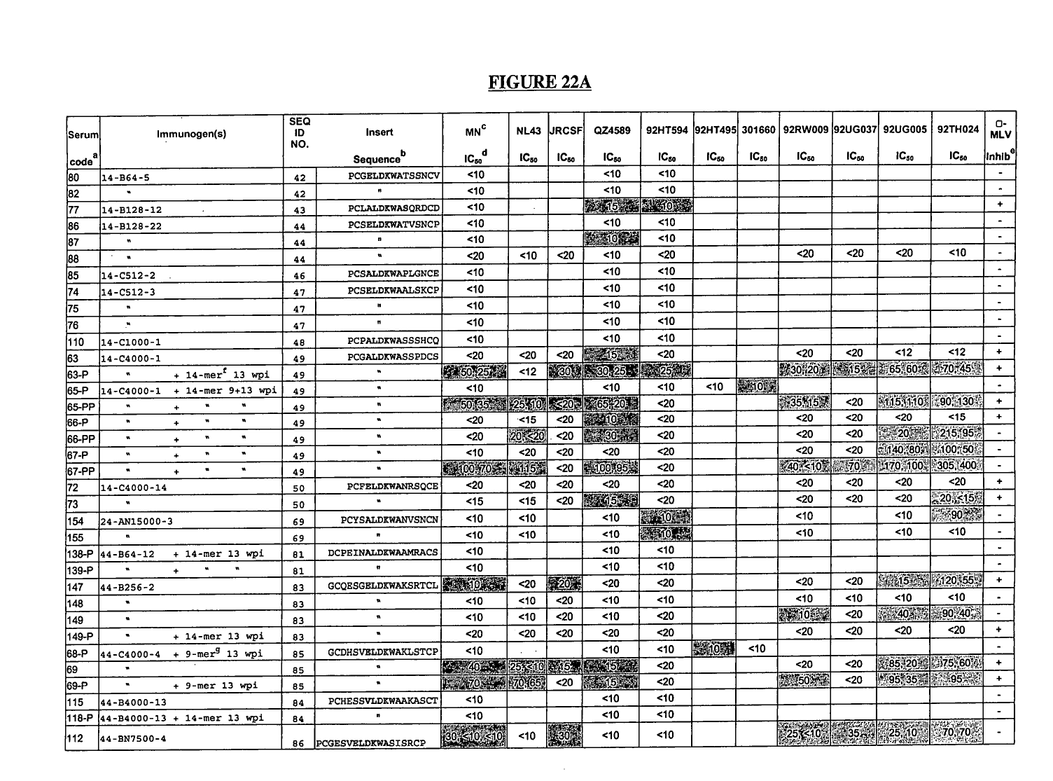

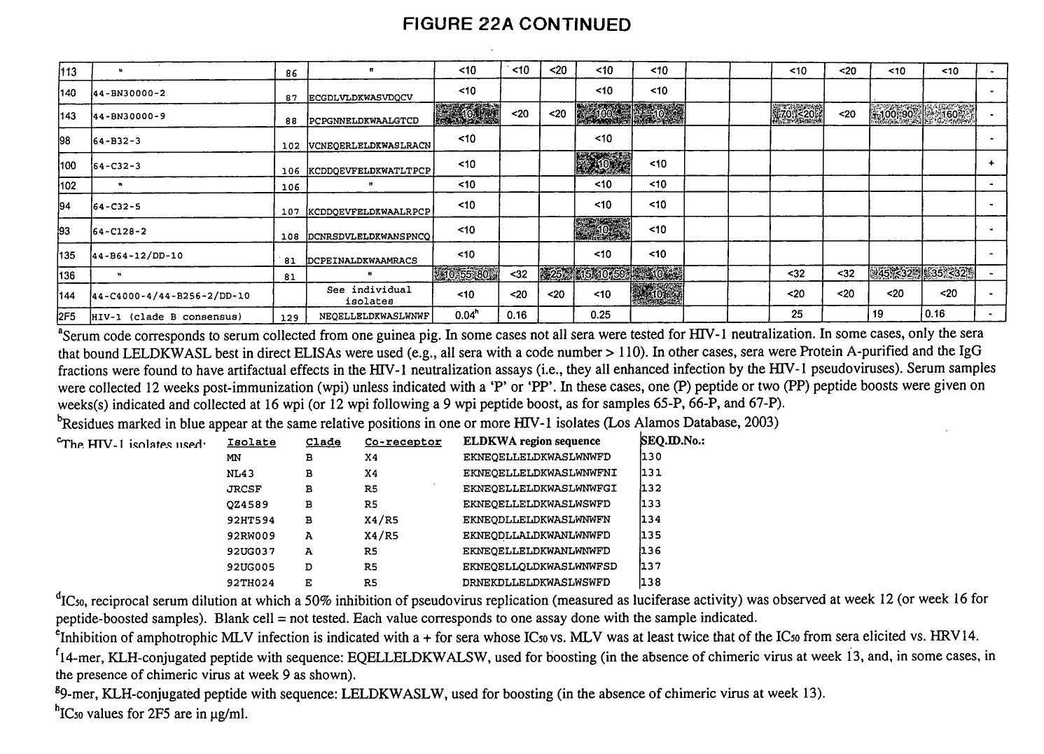

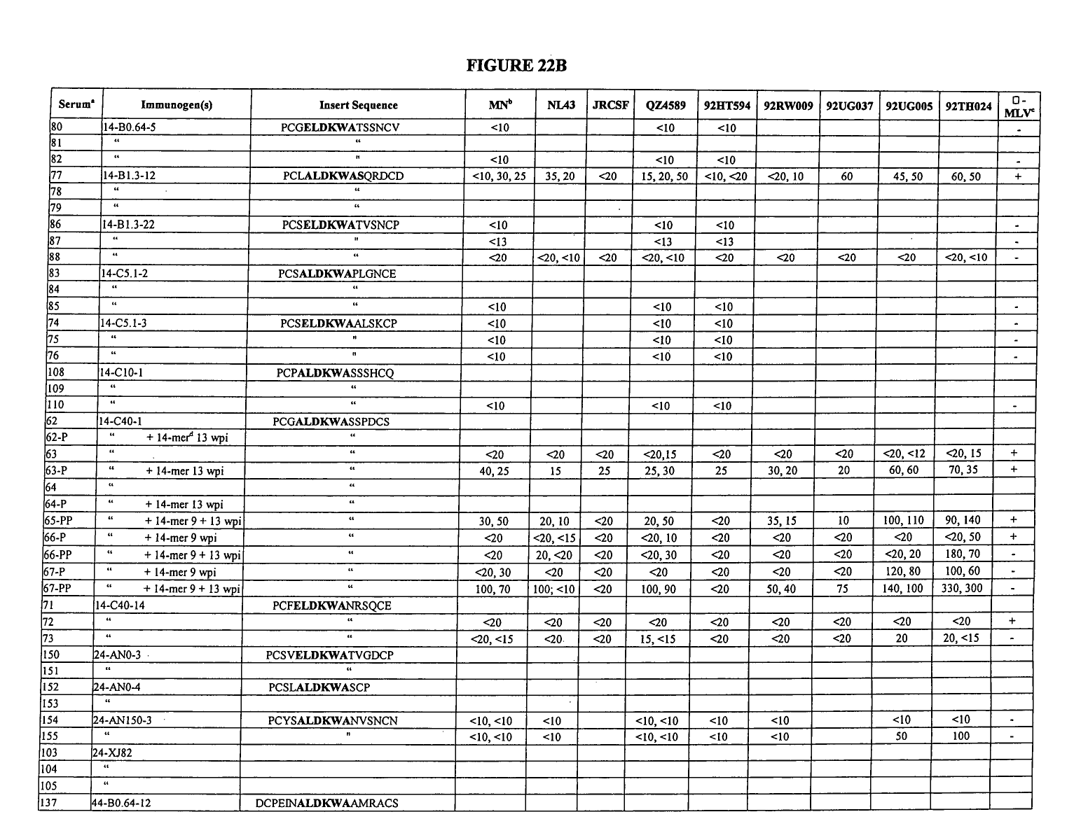

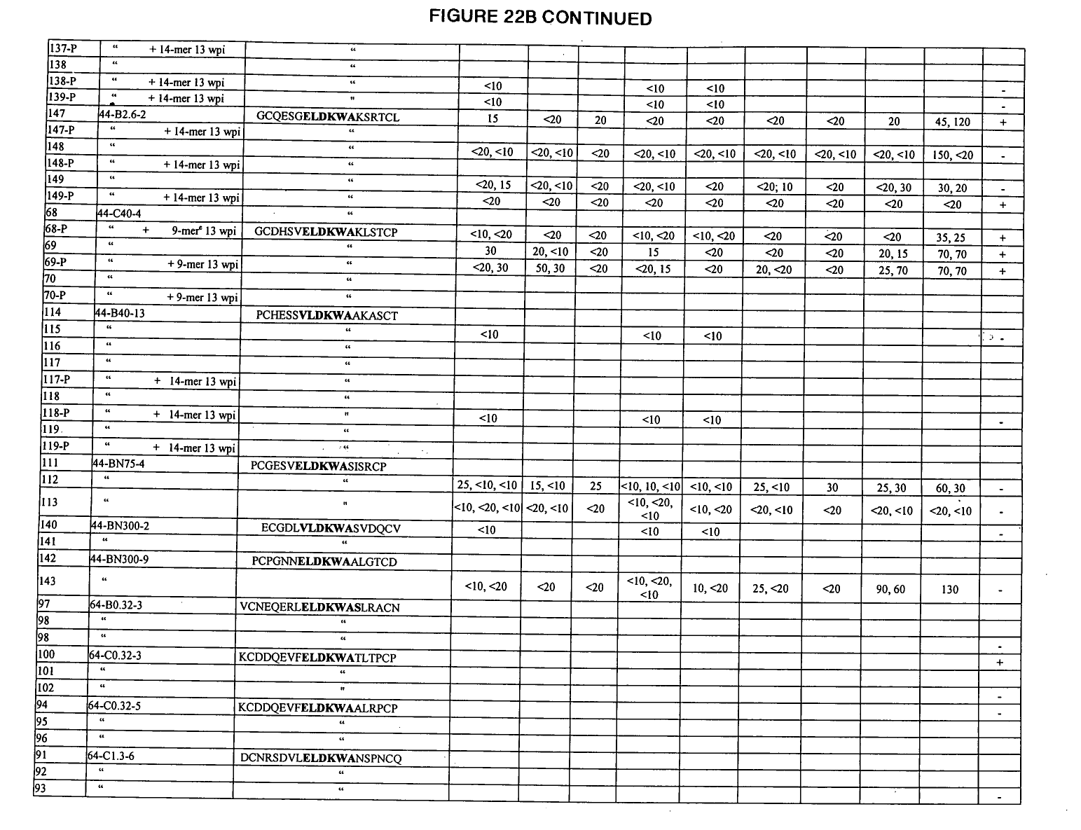

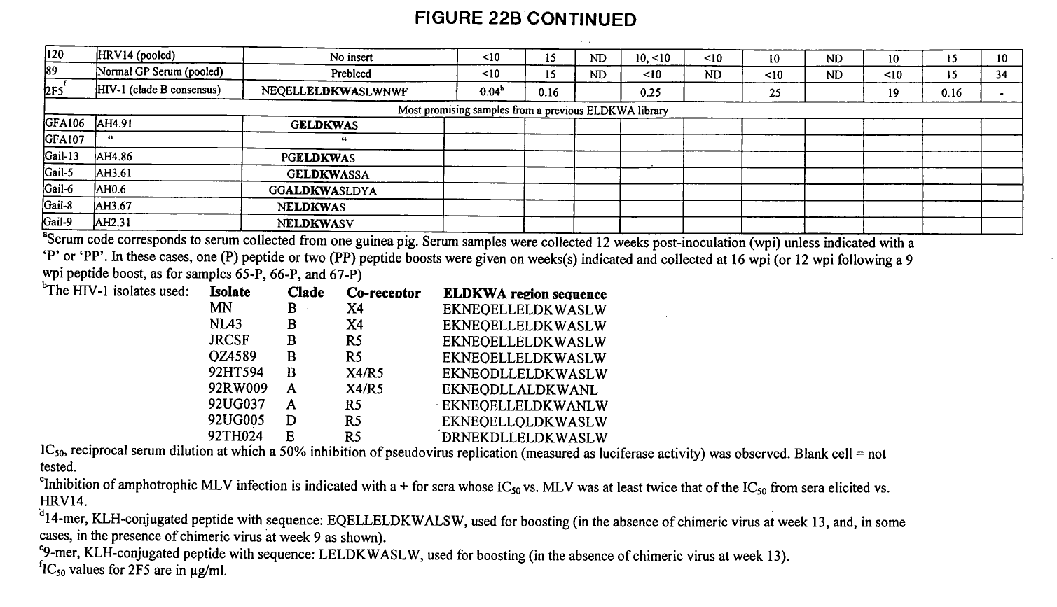

The LELDKWASL peptide-binding activity of the immune sera was compared to the ELISA and microtiter neutralization assays values obtained for the corresponding chimeras in hopes of establishing the existence of some relationship between antigenicity and immunogenicity (FIG. 20). The majority of the chimeric viruses (31/33) that elicited sera with IgG fractions capable of binding to the LELDKWASL peptide with serum 50% titers of ≧100 (FIG. 20) were also able to bind to immobilized 2F5 (ELISA titer, FIG. 20) or be neutralized by 2F5 (IC₅₀, FIG. 20) with titers of >10 for each assay. One case was observed in which the titers of both antigenicity assays for one chimera, 24-AN 15000-3, were low (<10) compared to the rest of the chimeras selected for immunization of guinea pigs, yet the sera elicited by it in one of the immunized animals had a good ability to bind LELDKWASL (titers>100). This chimera had been identified as having improved antigenicity compared to its parent unselected pool. For this reason, there were some expectations for positive immunogenicity results. The tendency to this point indicated that the antigenicity assays had the ability to help identify chimeric viruses that would elicit sera reactive to the ELDKWA epitope. It was expected that the sera with the highest reactivity to LELDKWASL peptide would also be capable of neutralization of HIV-1 isolates.

2. Ability of the Sera Elicited by HRV14:HIV-1 gp41 ELDKWA Chimeric Viruses to Neutralize HIV-1

The immune sera that showed reactivity to LELDKWASL peptide were tested for neutralizing activity against HIV-1 laboratory and primary isolates in a recombinant HIV-1 pseudovirus assay based on work reported by Richman et al. (Richman et al., 2003). The results shown here reflect the activity of non-purified immune sera only, as the IgG purified serum fractions that were tested for HIV-1 neutralization all showed enhancement of cell infection. Not all sera were tested against the same number of isolates due to limitations in sample availability; however, in all cases at least one TCLA and one primary isolate were tested. Twenty-four of the 47 sera tested were able to neutralize one or more presudoviruses of HIV-1, including at least one primary isolate at reciprocal serum dilutions ranging from 10 to 400. The set of HIV-1 neutralizing sera was elicited from 12 HRV14:HIV-1 gp41 ELDKWA chimeras, and from two cocktails of at least one ELDKWA chimera plus an HRV14:HIV-1 chimera that contains a V3-loop based epitope named DD1O (described in Smith et al., 1998).

The highest neutralization titers were obtained with sera elicited by chimeras 14-C4000-1 boosted with a 14-amino acid ELDKWA-based peptide, 44-B256-2, and 44-BN3000-9. The serum sample derived from immunization with 14-C4000-1 alone (serum 63) could only neutralize one isolate at a reciprocal neutralization titer of 15, while the sample elicited after one 14-mer peptide boost (63-P) had neutralizing ability against multiple isolates and at higher titers (up to 70). What is more, the sera elicited by 14-C4000-1 boosted at 9 and 13 weeks post-immunization (wpi) with the 14-mer peptide (sera 65-PP, 66-PP, and 67-PP) had the ability to neutralize several HIV-1 isolates at reciprocal dilution titers up to 400. In the case of chimera 14-C4000-1, the use of the peptide boost appeared to have a synergistic effect in the neutralizing ability of the sera obtained. It could also be observed that chimeras containing inserts with larger N-terminal linkers, such as 44-B256-2 (sera 147-149), and 44-C4000-4 (serum 69) were not associated with increased neutralizing capabilities as a function of the peptide boost (sera 149-P and 69-P). While this study did not include any peptide-only immunizations, it appears that peptide boosts did not, on their own, elicit an immune response as it was observed that there are cases in which a peptide boost was used with chimeras 44-B4000-13, and 44-B64-12, yet no neutralizing sera were elicited (sera 118-P, 138-P, and 139-P).

Finally, in order to find out if the screening methodologies used to asses the ability to bind and be neutralized by 2F5 in order to select the most antigenic chimeras had a good ability to predict immunogenicity, a relationship among the ability of the chimeras to bind 2F5 (ELISA titer), the ability of 2F5 neutralize the chimeras (2F5 titer), and the ability of the corresponding sera to neutralize HIV-1 was sought. It can be observed that 11/20 chimeras in the group were immunogenic in at least one guinea pig (designated as immunogenic if they are able to elicit HIV-1 neutralizing sera in at least one of the immunized guinea pigs), 9 of the 11 immunogenic chimeras presented ELISA titers≧10, and 4/9 of these had 2F5 neutralization titers>10. Only 3 of the 11 immunogenic chimeras had titers≧10 for both assays (44-BN7500-4, 44-BN-30000-9, and 64-C32-3). Samples with titers≧10, the ELISA titers provided more than twice as much prognostic value (81%; 9/11) as the 2F5 titers (36%; 4/11) for identifying possible immunogenic chimeras. In contrast, of the group of 9 non-immunogenic chimeras, 7 had ELISA titers≧10, and 6 had 2F5 titers≧10, which indicated that the assays or at least a cutoff value of 10 still had a high probability of identifying false negatives. Setting a higher value in the titers of these assays could lead to the identification of groups containing a higher percentage of immunogenic chimeras, and less false positives, although it could also lead to losing some immunogenic viruses that for some reason do not rank

### Example 6

### Crystallization of Chimeric Virus

Chimera 14-C4000-1 was purified and hanging drops were set up using three screening conditions based on varying concentrations of: (1) ammonium formate, and 50-150 mM Na HEPES, pH 7.4, (2) Na, K tartrate, 50-150 mM Na HEPES, pH, and (3) 10 mM Tris-HCl, pH 5 7.2, 20 mM CaCl₂, and 0.5% PEG 8000. The drops consistently showed precipitation in all conditions tested, except for 0.4 M Na,K Tartrate, 75 mM Na HEPES, pH 7.5. Two crystals of approximately 0.1×0.08×0.08 mm³ were obtained, and one with dimensions of 0.2×0.15×0.15 mm³. The latter crystal was taken to the Cornell High Energy Synchrotron Source (CHESS) F1 beamline and data were collected. The initial scaling and processing of the data indicated the crystal belonged to space group P22₁2; however, the mosaicity was consistently high (0.8°) and scaling was problematic. Previous analyses of a V3-loop based chimera that belonged to this space group had been difficult to solve.

As the space group seemed to relate to the crystallization conditions, efforts to grow crystals of chimera 44-C4000-4 were only done using screenings within which chimeric viruses crystals had been obtained and solved previously (Ding et al., 2002). This screening yielded showers of microcrystals using 2-2.5 M ammonium formate, 0.075-0.15 M Na HEPES, pH 7.5.

### Example 7

### Immunoselection of Chimeric Viruses

Immunoselection has previously been shown to be useful in enriching for chimeric rhinoviruses presenting V3 loop-like epitopes in ways capable of eliciting neutralization of HIV-1 (Smith et al., 1994; Resnick et al., 1995; Smith et al., 1998). This work was performed under the assumption that viruses that bound most tightly to the anti-HIV-1 neutralizing mAb 2F5 would be among those whose ELDKWA epitopes structurally best resembled those of HIV in the conformational state that promoted binding and neutralization by 2F5. It was further hypothesized that the structural similarity would favor the chances that the chimeric viruses would be able to elicit antibodies that had the potential to neutralize HIV like 2F5 (or conceivably even more effectively). Increasing the stringency of the selection was expected to increase the chance of enriching for and identifying the immunogens most conformationally similar to that which elicited 2F5. Thus, a competing peptide (with the sequence LELDKWASL) was added and/or the amount of immobilized 2F5 was decreased for immunoselection, and chimeric viruses were obtained that bound 2F5 with high affinity and with greater sensitivity to neutralization by 2F5 than unselected viruses (treated both as virus pools and as individual isolates; Table II and FIGS. 11-16).

As an additional means to optimize the imunoselection process, an alternative approach for eluting the pool of chimeric viruses enriched for binding to 2F5 was developed. In previous studies and in some of the pools generated in this study (FIG. 2), the chimeric viruses from the immobilized 2F5 were “eluted” by adding HeLa cells and allowing them to become infected with the viruses, then harvesting the viruses as lysates from the cells. Considering that the most tightly bound or otherwise neutralized viruses might not productively infect the cells, separate elutions were performed using high concentrations of LELDKWASL peptide instead of cells (for subsets of Pools II and III; FIG. 7). The chimeras that passed through a competitive immunoselection round and then to a round with no competition but with peptide elution (44-BN7500-4, 44-BN30000-2, BN-30000-9) turned out to be some of the most antigenic of the whole group of immunoselected chimeras (being the most sensitive to neutralization by 2F5; Table II and FIG. 15). These chimeras also were unique, as their sequences were not found in other groups. It is possible that cell elution would not have been able to result in the isolation of some of these viruses, as they may have been more effectively neutralized by the antibody, preventing their propagation in cells. This agrees with the observation that these chimeras were strongly neutralized with minimal concentrations of 2F5 (20 ng/ml or less; FIG. 15 and Table II).

### Example 8

### Sequence Characterization of Chimeric Viruses

The chimeras isolated had inserts of up to 22 foreign residues. There were some residues, like Trp, normally found adjacent to the ELDKWA epitope, that were not present in any of the live chimeras analyzed, even though Trp had been biased to be represented in 50% of chimeras at two positions C-terminal to ELDKWA (FIG. 5A-B; FIG. 6). Apparently, Trp has never been found among the live sequenced chimeric rhinoviruses that present other HIV epitopes in previous work. The sequencing data obtained at the plasmid level (Table 7) does not show any Trp at these positions either, however, the number of samples is too small to make any conclusions about it, and the residues at the corresponding positions are not defined for two of the eight sequenced samples (Table 7). The sequences of the inserts found in live viruses suggests that the presence of the bulky residue at positions close to the ones that link the foreign insert with HRV14's capsid may present a problem for virus viability. There is a Trp present in the middle of the epitope that was displayed in 100% of the sequenced isolates. The reason for this may be that Trp in this position is sufficiently far from the HRV14 natural sequence, giving an opportunity for the rest of the linker residues to confer proper stability and balance to allow virus viability. The most remarkable feature of the sequence analysis was the high prevalence of Pro (61%) at the N-terminal randomized linker position among all the sequenced chimeras. It was hypothesized that this unintended bias was probably needed for the proper incorporation of the foreign insert into the capsid of HRV14 in order to satisfy virus viability. Analysis using molecular modeling software supported this hypothesis. A crystal structure of a V3-loop epitope HRV14 chimeric virus previously determined revealed the presence of a β-turn at residue Ser158 (Ding et al., 2002). In the case of the ELDKWA chimeras created in this research, Ser158 of HRV14 was excluded from the design, leaving at the “turn” position the N-terminal randomized linker encoded by the library design. 61% of the sequenced live chimeras had a Pro at this position, and 16% had a Gly; therefore, the most prevalent residues at this position fully randomized by design are turn-forming residues. The turn at this position may be required to minimally perturb the rhinovirus capsid structure. It could be observed by modeling that a replacement with other residues such as Glu would still allow the turn to form, however the energy required for it to keep this conformation is larger. Therefore, it might be more economical for the virus to have a small, uncharged residue at this position to allow for turns for the foreign insert to be accommodated into VP2. The molecular modeling together with the MN-III-2 structural data indicate that the foreign insert adopts a series of turns to fold in and out of the rhinovirus VP2. This information suggests that the presence of Ser at several flanking positions in the chimeras described (in which it was not biased by design, particularly at positions −2, N-terminal to ELDKWA and +3, C-terminal to ELDKWA), might also favor turns at this site, as this is a non-bulky residue that can have both hydrophobic and hydrophilic nature.

An analysis of sequences of 31 immunoselected chimeras did not reveal any specific patterns for types of residues found to be directly flanking the epitope, both in terms of showing preferences for residues that naturally occur on HIV-1 at the corresponding positions or in terms of the frequencies of any other particular amino acids. Most of the immunoselected chimeras had a C-terminal linker containing 4 biased residues. This can be attributed to the fact that libraries with this linker size were largely represented (constituting 33% of the total clones sequenced) due to the contamination with C-terminal oligonucleotides encoding 4 biased residues.

A recent analysis of ELDKWA-based peptides with high affinity to 2F5 shows that, as has been mentioned before, the core epitope is DKW, and additional residues, though important, are there to provide the appropriate structural frame for 2F5 to recognize the epitope but do not provide additional contacts with the antibody (Barbato et al., 2003). The residues flanking the core epitope in immunoselected chimeras may serve to provide the most immunogenic presentation when the epitope is transplanted into a different structural environment, as in this case in the VP2 of HRV14. The specific combinations of residues flanking ELDKWA in the HRV 14 immunoselected chimeras may be a combination of the need for virus viability and epitope exposure. Therefore, even though some of the most immunogenic chimeras did not have many flanking residues similar to the native HIV-1 residues, the residues present might provide in this particular environment the appropriate structural background features for the core epitope to be presented in an immunogenic (and viable) conformation.

### Example 9

### Antigenicity and Immunogenicity of Chimeric Viruses HRV14:HIV-1 gp41 ELDKWA Libraries

Previous reports (Coeffier et al., 2000; Liao et al., 2000; Joyce et al., 2002; McGaughey et al., 2003) have suggested that antigenicity (recognition by pre-existing antibodies) is a necessary but insufficient criterion for obtaining immunogenicity (the ability to elicit de novo production of neutralizing antibodies). However, it has been shown that immunogens that are more antigenic will have better odds of eliciting an antibody response than immunogens that are not recognized at all by antibody (Coeffier, et al., 2000; Liao et al., 2000; Joyce et al., 2002; McGaughey et al., 2003).

For vaccine purposes, it is essential to identify chimeric viruses that are immunogenic.

However, there are no high-throughput assays for directly screening large numbers of potential immunogens, so indirect assessments must be used. A combination of assays has been chosen to assess antigenicity as a practical means to enrich for viruses that at least interact well with antibody (in this case using 2F5-binding and 2F5-neutralizing assays). One goal of these endeavors has been to develop an antigenicity profile that might have predictive potential for immunogenicity so the immunization of animals can be performed with the antigens most likely to result in the production of relevant neutralizing antibodies.

Two types of neutralization assays have been used to assess antigenicity: a microtiter neutralization assay, and a plaque reduction assay (Smith et al., 1994; Smith et al., 1998). There were no particular advantages found for using the two different neutralization assays, so this work involved only the 2F5 microtiter neutralization assay and combined it with a number of cell-free binding assays in which the ability of the viral particle to be recognized and bound by 2F5 would be assessed regardless of the ability of the virus to infect cells. The 2F5 microtiter neutralization assay and ELISAs did reveal the trend that the immunoselected clones were more strongly antigenic than their parent (unselected) pools. The most antigenic viruses were those used for the efforts to obtain appropriately immunogenic viruses. Since the 2F5 neutralization and ELISA titers did not always correlate, it was decided to study the isolates that scored well in one or both of the assays.

The sera obtained from guinea pigs were analyzed for their abilities to bind to the peptide of sequence LELDKWASL (which was found to bind to 2F5 nearly 100-fold more tightly in an ELISA than the shorter ELDKWAS peptide; Tian et al., 2002). The fact that some of the IgG fractions obtained could bind LELDKWASL nearly 50% as well as the same amount of 2F5 IgG (Table III) is significant in terms of how immunogenic the chimeras were. In addition, seven of 19 LELDKWASL-binding sera tested recognized a lactam-constrained ELDKWA peptide, designated Lac-1, designed to have a backbone conformation like that of the ELDKWAS peptide complexed with 2F5 Fab in the crystallographic structure determined by Pai et al (2000). The Lac-1 peptide binds 2F5 two times more tightly than the LELDKWASL peptide (Tian, 2003). The observation that some of the immune sera elicited by the chimeras bound more tightly to Lac-1 than to LELDKWASL (Table III) provides further indication that the conformation of the ELDKWA region of the corresponding chimeras may be immunologically relevant.

It is still possible to adjust stringency conditions in such an assay, perhaps adding competition, in order to rule out samples that are poorly immunogenic. These results indicate that antigenic samples have a better probability of being immunogenic than non-antigenic samples. Although there is a margin of error in the assays performed, they are still useful to identify the best vaccine candidates, and, perhaps when a large number of samples is being handled, the cut-off values to qualify samples as antigenic or non-antigenic should be raised. In this particular case, the focus was set on the antigenicity of the immunoselected clones compared to the ones of the parent pools they had been isolated from. The ones whose 2F5 binding and sensitivity to neutralization had increased with respect to the parent pool were classified as “more antigenic”.

### Example 10

### Epitope-Based Vaccine

An effective vaccine against HIV will have to be able to stimulate both arms of the immune system in order to be able to control or prevent infection. Throughout years of HIV-vaccine directed research, it has been difficult to find immunogens that would elicit the production of potent and cross-neutralizing antibodies (Muster et al., 1995; Joyce et al., 2002; McGaughey et al., 2003), and attention has been put into developing vaccines that will stimulate CTL responses instead. However, the isolation of potent and cross-neutralizing human monoclonal antibodies (Muster et al., 1993; Burton et al., 1994; Conley et al., 1994; Purtscher et al., 1994; Trkola et al., 1996; Zwick et al., 2001) and the use of these antibodies in studies of passive and therapeutic immunization has led to results that indicate that neutralizing antibodies play a crucial role in the prevention and control of HIV infection, viral load set point, and prognosis of infected individuals (Mascola et al., 1999; Zwick et al., 2001; Lewis et al., 2002; Ferrantelli et al., 2003; Ruprecht et al., 2003; Schmitz et al., 2003;).

The mAb 2F5 is one of the few human antibodies with cross-neutralizing activity known to date (Burton et al., 1994; Trkola et al., 1995; Li et al., 1997; Mascola et al., 1997; Zwick et al. 2001a). This antibody has also been tested in vivo, conferring partial or full protection from infection in animals either alone or combined with other neutralizing monoclonals (Mascola et al., 1997; Mascola et al., 1999; Zwick et al., 2001b; Ferrantelli et al., 2003; Ruprecht et al., 2003). The epitope recognized by mAb 2F5 is the sequence ELDKWA, located near the C-terminal end of the gp41 ectodomain, close to the transmembrane domain (Muster et al., 1993; Conley et al., 1994). This sequence is highly conserved in most HIV-1 isolates, which makes it a promising target for vaccine development. However, until now, no one has succeeded in displaying the ELDKWA epitope in immunogenic conformations that lead to the production of neutralizing antibodies (Joyce et al., 2002; McGaughey et al., 2003).

The chimeric viruses generated in this study were able to elicit neutralizing antibodies against HIV-1 (both primary and laboratory isolates; Velasco et al., 2004). While some of the neutralization titers observed are modest, all other published efforts have shown no neutralization of primary isolates at all (Muster et al., 1994; Xiao et al., 2000; Joyce et al., 2002; McGaughey et al., 2003), even though, in some cases, high titers of ELDKWA-reactive antibodies have been obtained. The titers obtained in this experiment might be sufficient to confer some degree of protection. For one, it has been observed that neutralizing antibody levels even below a protective threshold for preventing infection can substantially reduce plasma viremia (Mascola et al., 2003). Additionally, the observed titers could be significant for protection at mucosal surfaces, as it has been demonstrated that 2F5 IgA or IgM (that sometimes had to be concentrated 10 times as much as the corresponding IgG to achieve 50% neutralization of HIV primary isolates in tissue culture) were capable of interfering with HIV-1 entry across a mucosal epithelial layer in vitro (Wolbank et al., 2003). Additionally, even in serum, titers as low as 1:38 have been reported to confer protection to macaques from SHIV_{DH12} infection after they had been passively administered IgG from chimpanzees inoculated with HIV_{DH12} (Nishimura et al., 2002). Furthermore, the calculated serum concentrations of HIV-specific IgG in these protected animals ranged from 2.2-2.6 mg/ml. Based on the average IgG concentrations observed in the guinea pig sera elicited by the ELDKWA chimeras (3-3.5 mg/ml) and on the calculation that up to 46% of this IgG might be specific for ELDKWA (1.5 mg/ml), neutralizing sera at dilution titers as low as 1:25 (for the samples with the highest ELDKWA reactivity; comparing with the 1:38 protective titer reported above) might be protective. It should also be taken into account that a vaccine that is able to reduce viral load (even if it is not fully protective) will have an important impact on wellness and transmission rate (reviewed by Pope and Haase, 2003).

Although the guinea pig system is useful for screening of a large number of samples, there are some disadvantages that have to be overcome once a particular set of immunogenic viruses is identified as having potential of being developed into a vaccine. It is particularly important to note that guinea pigs are not permissive for replication of HRV14, making it likely that neutralizing titers would be higher in humans. In fact, a mouse model permissive for replication of HRV is currently being developed (Harris and Racaniello, 2003; Tuthill et al., 2003) that could prove extremely valuable for obtaining more relevant neutralizing antibody responses.

### Example 11

### Multiple Epitope Vaccine

One of the big challenges for the development of an effective anti-AIDS vaccine is the high propensity of HIV-1 to mutate. An effective vaccine will have to be somewhat heterogeneous in order to apply strong pressure on several targets at a time (Ho and Huang, 2002). One way to approach this challenge is to use immunization cocktails of two or more chimeras, working with the hypothesis that these would have an additive or synergistic effect in eliciting a neutralizing response. When this experiment was done with chimeric HRVs, two out of three sera elicited by cocktails of chimeras that were tested for HIV neutralization could neutralize one or more HIV-1 isolates. The LELDKWASL-binding proportion of antibodies contained in the neutralizing samples are on different extremes (serum 136=0.46, serum 144=0.04; Table III), suggesting that the neutralization ability of serum 136 is due to LELDKWASL-reactive antibodies, first because it has a high proportion of those (46%), and second, because serum 135 (which was elicited by the same cocktail mixture, 44-B64-12/DD-10) had low LELDKWASL reactivity (0.05; Table III), and was not capable of HIV neutralization. The extremely different response to the same immunogen by two different animals seems to indicate animal-to-animal variation. Serum 144 (elicited by a cocktail of 44-C4000-4/44-B256-2/DD-10) had a very low LELDKWASL-binding titer, yet was capable of neutralizing HIV-1. Although this would lead us to suggest immediately that the V3 loop reactive antibodies are responsible for this neutralizing ability, it is also important to observe that both ELDKWA chimeras included in this cocktail were able to elicit neutralizing sera on their own (samples 147, 149, and 69), and in combination with peptide boosts (68-P, 69-P).

### Example 12

### Peptide Boost

An additional alternative incorporated in this work to elicit a neutralizing response against HIV was the use of ELDKWA-based peptides to boost some of the animals immunized with ELDKWA chimeras. It was hypothesized that these peptides, a 14-mer containing the sequence EQELLELDKWASLW, which was suggested as an extended version of the 2F5 epitope that conferred better antibody binding (Parker et al., 2001), and a 9-mer with the sequence LELDKWASL that binds 2F5 with high affinity (Tian et al., 2002), would have an additive or synergistic effect on the neutralizing response elicited by the ELDKWA chimeras.

The peptide boosts had a remarkable additive effect in the ability to elicit neutralizing sera, most notably in chimera 14-C4000-1, in which an increase in neutralization titers (up to a 5-fold) and cross-reactivity could be observed when comparing the sera elicited by the chimera alone (serum 63) and the serum elicited after the peptide boost (63-P). It was also observed that sera 65-PP, 66-PP, and 67-PP that reflect responses from two consecutive peptide boosts showed increased neutralization titers (up to 5-fold) and cross-reactivity compared to the sera drawn after only one peptide boost (65-P, 66-P, and 67-P). In chimera 44-C4000-4, the peptide boost did not have a dramatic effect, but it showed a slight increase (up to almost 3-fold) in the neutralization titers against two isolates. It can then be concluded that, in general, the peptide boost had a positive effect on the ability to increase the neutralizing response to chimeric viruses. The one exception found in which the serum elicited after the peptide boost turned out to be non-neutralizing while the sample drawn before the peptide boost was neutralizing (149-P and 149) might be due to a particularly short-lived response in the animal.

This invention may be embodied in other forms or carried out in other ways without departing from the spirit or essential characteristics thereof. The present disclosure is therefore to be considered as in all respects illustrative and not restrictive, the scope of the invention being indicated by the appended Claims, and all changes which come within the meaning and range of equivalency are intended to be embraced therein.

The following is a list of documents related to the above disclosure and particularly to the experimental procedures and discussions. The documents should be considered as incorporated by reference in their entirety.

### REFERENCES

Arnold, E., Erickson, J. W., et al. (1984). Virion orientation in cubic crystals of the common cold virus HRV 14. Journal of Molecular Biology 177: 417-430.
Arnold, E. & Arnold, G. F. (1990). Virus structure and the AIDS problem: strategies for antiviral design based on structure. Use of X-Ray Crystallography in the Design of Antiviral Agents, Academic Press, Inc.: 283-295.
Arnold, G. F., et al. (1994). Design and construction of rhinovirus chimeras incorporating immunogens from polio, influenza, and human immunodeficiency viruses. Virology 198: 703-708.
Arnold, G. F. & Arnold, E. (1999). Using combinatorial libraries to develop vaccines. ASM News 65(9): 603-610.
Barbato, G., E. et al. (2003). Structural analysis of the epitope of the anti-HIV antibody 2F5 sheds light into its mechanism of neutralization and HIV fusion. Journal of Molecular Biology 330(5): 1101-15.
Burton, D. R., et al. (1994). Efficient neutralization of primary isolates of HIV-1 by a recombinant human monoclonal antibody. Science 266: 1024-1027.
Burton, D. R. & Montefiori, D. (1997). The antibody response in HIV-1 infection. AIDS 11(Suppl A): 587-598.
Coeffier, E., et al. (2000). Antigenicity and immunogenicity of the HIV-1 gp41 epitope ELDKWA inserted into permissive sites of the MalE protein. Vaccine 19(7-8): 684-93.
Colonno, R. J., et al. (1988). Evidence for the direct involvement of the rhinovirus canyon in receptor binding. Proceedings of the National Academy of Sciences, U.S.A. 85: 5449-5453.
Conant, R. M. & Hamparian V. V. (1968). Rhinoviruses: basis for a numbering system 1. HeLa cells for propagation and serologic procedures. Journal of Immunology 100(1): 107-113.
Conley, A. J., et al. (1994). Neutralization of divergent human immunodeficiency virus type 1 variants and primary isolates by IAM-41-2F5, an anti-gp41 human monoclonal antibody. Proceedings of the National Academy of Sciences, U.S.A. 91: 3348-3352.
Devlin, J. J., et al. (1990). Random peptide libraries: a source of specific protein binding molecules. Science 249: 404-406.
Ding, J., et al. (2002). Crystal structure of a human rhinovirus that displays part of HIV-1 V3 loop and induces neutralizing antibodies against HIV-1. Structure 10: 999-1011.
D'Souza, M. P., et al. (1995). Neutralization of primary HIV-1 isolates by anti-envelope monoclonal antibodies. AIDS 9: 867-874.
D'Souza, M. P., et al. (1997). Evaluation of monoclonal antibodies to human immunodeficiency virus type 1 primary isolates by neutralization assays: performance criteria for selecting candidate antibodies for clinical trials. Journal of Infectious Diseases 175: 1056-1062.
Ferrantelli, F., H et al. (2003). Post-exposure prophylaxis with human monoclonal antibodies prevented SHIV89.6P infection or disease in neonatal macaques. AIDS 17(3): 301-9.
Grant, S. G. N., et al. (1990). Differential plasmid rescue from transgenic mouse DNAs into Escherichia coli methylation-restriction mutants. Proceedings of the National Acadey of Sciences 87(June): 4645-4649.
Hendrickson, E. & Hatfield, T. (1990). Double stranded DNA miniprep protocol for the Genesis 2000 DNA Analysis System. Genesis™ 2000 DNA Analysis System Application Brief 16 (February 1990): 1-2.
Harris, J. R. & Racaniello, V. R. (2003). Changes in rhinovirus protein 2C allow efficient replication in mouse cells. Journal of Virology 77(8): 4773-80.
Ho, D. D. & Huang, Y. (2002). The HIV-1 vaccine race. Cell 110(2): 135-8.
Joyce, J. G., et al. (2002). Enhancement of alpha-helicity in the HIV-1 inhibitory peptide DP 178 leads to an increased affinity for human monoclonal antibody 2F5 but does not elicit neutralizing responses in vitro. Implications for vaccine design. Journal of Biological Chemistry 277(48): 45811-20.
Kessler, J. A., et al. (1997). Recombinant human monoclonal antibody IgG1b12 neutralizes diverse human immunodeficiency virus type 1 primary isolates. AIDS Research and Human Retroviruses 13(7): 575-581.
Korber, B., B. Hahn, B., Foley, B., Mellors, J. W., Leitner, T., Myers, G., McCutchan, F., Kuiken, C. L. Eds. (1998). Human Retroviruses and AIDS. Los Alamos, N.M., Los Alamos National Laboratories.
Korber, B., B. Hahn, B., Foley, B., Mellors, J. W., Leitner, T., Myers, G., McCutchan, F., Kuiken, C. L. Eds. (2002). Human Retroviruses and AIDS. Los Alamos, N.M., Los Alamos National Laboratories.
Lewis, A. D., et al. (2002). Generation of neutralizing activity against human immunodeficiency virus type 1 in serum by antibody gene transfer. Journal of Virology 76(17): 8769-75.
Li, A., Baba, et al. (1997). Synergistic neutralization of a chimeric SIV/HIV type 1 vitus with combinations of human anti-HIV type 1 envelope monoclonal antibodies or hyperimmune globulins. AIDS Research and Human Retroviruses 13(8): 647-656.
Liao, M., et al. (2000). Induction of high level of specific antibody response to the neutralizing epitope ELDKWA on HIV-1 gp41 by peptide-vaccine. Peptides 21(4): 463-8.
Mascola, J. R., et al. (1997). Potent and synergistic neutralization of human immunodeficiency virus (HIV) type 1 primary isolates by hyperimmune anti-HIV immunoglobulin combined with monoclonal antibodies 2F5 and 2G 12. Journal of Virology 71(October): 7198-7206.
Mascola, J. R., et al. (1999). Protection of macaques against pathogenic simian/human immunodeficiency virus 89.6D passive transfer of neutralizing antibodies. Journal of Virology 73: 4009-4018.
Mascola, J. R., et al. (2003). Cellular immunity elicited by human immunodeficiency virus type 1/simian immunodeficiency virus DNA vaccination does not augment the sterile protection afforded by passive infusion of neutralizing antibodies. Journal of Virology 77(19): 10348-56.
McGaughey, G. B., et al. (2003). HIV-1 vaccine development: constrained peptide immunogens show improved binding to the anti-HIV-1 gp41 MAb. Biochemistry 42(11): 3214-23.
McKeating, J. A., et al. (1996). Chimeric viruses expressing primary envelope glycoproteins of human immunodeficiency virus type 1 show increased sensitivity to neutralization by human sera. Virology 220: 460-480.
Miller, J. H. (1972). Experiments in Molecular Genetics. Cold Spring Harbor, N.Y., Cold Spring Harbor Laboratory.
Muster, T., et al. (1993). A conserved neutralizing epitope on gp41 of human immunodeficiency virus type 1. Journal of Virology 67(11): 6642-6647.
Muster, T., et al. (1994). Cross-neutralizing activity against divergent human immunodeficiency virus type I isolates induced by the gp41 sequence ELDKWAS. Journal of Virology 68(6): 4031-4034.
Muster, T., et al. (1 995). Mucosal model of immunization against human immunodeficiency virus type 1 with a chimeric influenza virus. Journal of Virology 69(11): 6678-6686.
Nishimura, Y., et al. (2002). Determination of a statistically valid neutralization titer in plasma that confers protection against simian-human immunodeficiency virus challenge following passive transfer of high-titered neutralizing antibodies. Journal of Virology 76(5): 2123-30.
Pai, E. F., et al. (2000). Fab-epitope complex from the HIV-1 cross-neutralizing monoclonal antibody 2F5. International Patent Publication Number: WO 00/61618.
Parker, C. E., et al. (2001). Fine Definition of the epitope on the gp41 glycoprotein of human immunodeficiency virus type 1 for the neutralizing monoclonal antibody 2F5. Journal of Virology 75(22): 10906-10911.
Pear W S, et al. (1993). Production of high-titer helper-free retroviruses by transient transfection. Procedings of the National Academy of Sciences USA. 90(18): 8392-6.
Ponten J., Macintyre, E. H. (1968). Long term culture of normal and neoplastic human glia. Acta Pathologica et Microbiologica Scandinavica. 74(4): 465-486.
Pope, M. & Haase, A. T. (2003). Transmission, acute HIV-1 infection and the quest for strategies to prevent infection. Nature Medicine 9(7): 847-52.
Purtscher, M., et al. (1994). A broadly neutralizing human monoclonal antibody against gp41 of human immunodeficiency virus type 1. AIDS Research and Human Retroviruses 10(12): 1651-1658.
Purtscher, M., et al. (1996). Restricted antigenic variability of the epitope recognized by the neutralizing gp41 antibody 2F5. AIDS 10: 587-593.
Resnick, D. A., et al. (1995). Chimeras from a human rhinovirus 14-human immunodeficiency virus type 1 (HIV-1) V3 loop seroprevalence library induce neutralizing responses against HIV-1. Journal of Virology 69: 2406-2411.
Richman, D. D., et al. (2003). Rapid evolution of the neutralizing antibody response to HIV type 1 infection. Procedings of the National Academy of Sciences USA 100(7): 4144-9.
Ruprecht, R. M., et al. (2003). Antibody protection: passive immunization of neonates against oral AIDS virus challenge. Vaccine 21(24): 3370-3.
Sambrook, J., et al. (1989). Molecular cloning: a laboratory manual, 2nd ed, Cold Spring Harbor Laboratory Press.
Schmitz, J. E., et al. (2003). Effect of humoral immune responses on controlling viremia during primary infection of rhesus monkeys with simian immunodeficiency virus. Journal of Virology 77(2): 2165-73.
Sherry, B. (1985). Identification of neutralization antigens on human rhinovirus 14, University of Wisconsin, Madison, Wis. Ph.D. Thesis.
Sherry, B., et al. (1986). Use of monoclonal antibodies to identify four neutralization immunogens on a common cold picomavirus, human rhinovirus 14. Journal of Virology 57: 246-257.
Sherry, B. & Rueckert, R. R. (1985). Evidence for at least two dominant neutralization antigens on human rhinovirus 14. Journal of Virology 53: 137-143.
Smith, A. D., et al. (1994). Use of random systematic mutagenesis to generate viable human rhinovirus 14 chimeras displaying HIV-1 V3 loop sequences. Journal of Virology 68: 575-579.
Smith, A. D., et al. (1998). Human rhinovirus type 14:human immunodeficiency virus type 1 (HIV-1) V3 loop chimeras from a combinatorial library induce potent neutralizing antibody responses against HIV-1. Journal of Virology 72(1): 651-659.
Tian, Y., et al. (2002). Structure-affinity relationships in the gp41 ELDKWA epitope for the HIV-1 neutralizing monoclonal antibody 2F5: Effects of side-chain and backbone modifications and conformational constraints. Peptide Research 59: 264-276.
Tian, Y. (2003). Structure-based development of peptide vaccines against HIV-1 derived from a broadly neutralizing epitope on hiv-1 gp41. Rutgers, The State University of New Jersey. Ph.D. Thesis.
Trkola, A., et al. (1995). Cross-clade neutralization of primary isolates of human immunodeficiency virus type 1 by human monoclonal antibodies and tetrameric CD4-IgG. Journal of Virology 69: 6609-6617.
Trkola, A., et al. (1996). Human monoclonal antibody 2G12 defines a distinctive neutralization epitope on the gp120 glycoprotein of human immunodeficiency virus type 1. Journal of Virology 70(9): 1100-8.
Trkola, A., et al. (2001). Potent, broad-spectrum inhibition of human immunodeficiency virus type 1 by the CCR5 monoclonal antibody PRO 140. Journal of Virology 75(2): 579-88.
Tuthill, T. J., et al. (2003). Mouse respiratory epithelial cells support efficient replication of human rhinovirus. Journal of General Virology 84(Pt 10): 2829-36.
Velasco, P. K., et al. (2004). Broad neutralization of HIV-1 primary isolates elicited by gp41 ELDKWA-displaying human rhinoviruses selected from combinatorial libraries. AIDS Research and Human Retroviruses. In preparation.
Wolbank, S., et al. (2003). Characterization of human class-switched polymeric (immunoglobulin M [IgM] and IgA) anti-human immunodeficiency virus type 1 antibodies 2F5 and 2G12. Journal of Virology 77(7): 4095-103.
Xiao, Y., et al. (2000). Epitope-vaccine induces high levels of ELDKWA-epitope-specific neutralizing antibody. Immunology Investigation 29(1): 41-50.
Zhang, A., et al. (1993). Structure determination of antiviral compound SCH 38057 complexed with human rhinovirus 14. Journal of Molecular Biology 230: 857-867.
Zhang, A., et al. (1999). A disulfide-bound HIV-1 V3 loop sequence on the surface of human rhinovirus 14 induces neutralizing responses against HIV-1. Biological Chemistry: 365-374.
Zwick, M. B., et al. (2001)a. Broadly neutralizing antibodies targeted to the membrane-proximal external region of human immunodeficiency virus type 1 glycoprotein gp41. Journal of Virology 75(22): 10892-10905.
Zwick, M. B., et al. (2001 )b. Neutralization synergy of human immunodeficiency virus type 1 primary isolates by cocktails of broadly neutralizing antibodies. Journal of Virology 75(24): 12198-12208.

We claim:

1. An immunogenic composition comprising:
a. an isolated recombinant chimeric human rhinovirus, wherein the recombinant chimeric human rhinovirus comprises
i. a nucleic acid having a nucleotide sequence of a human rhinovirus encoding at least a portion of a human rhinovirus capsid;
ii. a heterologous nucleic acid having a nucleotide sequence encoding a chimeric region, wherein the chimeric region is at least in part expressed on the surface of the chimeric rhinovirus and is capable of participating in an immune reaction; and
b. a pharmaceutically acceptable carrier.

2. The composition according to claim 1, wherein the recombinant chimeric rhinovirus is comprising a nucleic acid or portion thereof having the following characteristics:
a. it encodes a polypeptide capable of forming at least a portion of a human rhinovirus capsid; and
b. it has the ability to hybridize under standard hybridization conditions to a nucleic acid sequence or complement thereof, capable of encoding the sequence shown in SEQ ID NO.: 1.

3. The composition according to claim 1, wherein the nucleotide sequence encoding the rhinovirus capsid encodes at least part of a rhinovirus neutralizing immunogenic site.

4. The composition according to claim 1, wherein the recombinant chimeric rhinovirus is viable.

5. The composition according to claim 1, wherein the recombinant chimeric rhinovirus is non-viable.

6. The composition according to claim 1, wherein the recombinant chimeric rhinovirus is biologically pure.

7. A plasmid capable of generating an infectious recombinant rhinovirus cDNA having the following characteristics:
a. it encodes a polypeptide capable of forming at least a portion of a rhinovirus capsid; and
b. it has the ability to hybridize under standard hybridization conditions to a nucleic acid sequence or complement thereof, capable of encoding the sequence shown in SEQ ID NO.: 1.

8. An isolated, infectious cDNA of the recombinant rhinovirus of claim 1.

9. The infectious cDNA of claim 8, incorporated within a vector.

10. The vector of claim 9, wherein said vector is a plasmid.

11. A prokaryotic host cell transformed with the vector of claim 9.

12. A eukaryotic host cell transformed with the vector of claim 9.

13. An isolated infectious RNA transcribed from the vector of claim 9.

14. An isolated cell line comprising the cDNA of claim 8.

15. An isolated recombinant human rhinovirus generated from the RNA of claim 13.

16. An isolated recombinant human rhinovirus produced from the cell line of claim 14.

17. A unit dose of an immunogenic composition comprising the virus of claim 15.

18. A unit dose of an immunogenic composition comprising the virus of claim 16.

19. A method of inducing an immune response comprising the step of administering a unit dose of the composition of claim 1 to a vaccinee.

20. The composition according to claim 1, wherein the recombinant chimeric rhinovirus is comprising a nucleic acid or portion thereof having the following characteristics:
a. it encodes a polypeptide capable of forming at least a portion of a human rhinovirus capsid; and
b. it has the ability to hybridize under standard hybridization conditions to a nucleic acid sequence or complement thereof, capable of encoding the sequence shown in SEQ ID NO.: 2.

21. The composition according to claim 1, wherein the recombinant chimeric rhinovirus is comprising a nucleic acid or portion thereof having the following characteristics:
a. it encodes a polypeptide capable of forming at least a portion of a human rhinovirus capsid; and
b. it has the ability to hybridize under standard hybridization conditions to a nucleic acid sequence or complement thereof, capable of encoding the sequence selected from the sequential group consisting of SEQ. ID NO.: 3 through SEQ. ID. NO.:118.

22. The composition according to claim 1, wherein the recombinant rhinovirus is constructed by inserting into the nucleotide sequence of a human rhinovirus encoding part of a neutralizing immunogenic site, a heterologous nucleotide sequence encoding a chimeric region, wherein the chimeric region is expressed on the surface of the chimeric rhinovirus and is capable of participating in an immune reaction.

23. The composition of claim 1, wherein the chimeric region is presented in the NIm-II portion of viral protein VP2.

24. The chimeric rhinovirus of claim 1, wherein the chimeric region is presented in the NIm-IA portion of viral protein VP1.

25. The chimeric rhinovirus of claim 1, wherein the chimeric region is of viral origin.

26. The chimeric rhinovirus of claim 11, wherein the viral origin of the chimeric region is a retrovirus.

27. The chimeric rhinovirus of claim 26, wherein the retrovirus is a human immunodeficiency virus.

28. The chimeric rhinovirus of claim 27, wherein the human immunodeficiency virus is selected from the group consisting of HIV-1 and HIV-2.

29. The chimeric rhinovirus of claim 28, wherein the chimeric region is presented at loop 2 of viral protein VP2 of NIm-II immunogenic site of HRV14.

30. The chimeric rhinovirus of claim 28, wherein the chimeric region is presented between from about amino acid 159 to about amino acid 161 of VP2.

31. A kit for producing the recombinant rhinovirus of claim 1 comprising
a. the infectious cDNA of claim 8; and
b. a coupled transcription and translation system.

32. The kit of claim 31, further comprising a cellular expression system.

33. The kit of claim 32, wherein the coupled transcription and translation system further comprises:
a. the nucleic acid of claim 8;
b. a eukaryotic cell free cell extract, wherein the extract is from either an animal or a plant cell
c. ribonucleotide triphosphates; and
d. RNA polymerase.

34. A method for generating the composition of claim 1 comprising
a. generating nucleic acid mixture comprising:
i. a nucleotide sequence of a human rhinovirus encoding at least a portion of a human rhinovirus capsid;
ii. a nucleotide flanking sequence 3′ to a chimeric sequence insertion site;
iii. a nucleotide flanking sequence site 5′ to the chimeric sequence insertion site, wherein the nucleotide sequences flanking the chimeric site comprise a pseudo random selection of nucleotides, capable of encoding a selection of amino acids;
iv. a heterologous nucleic acid chimeric insertion sequence comprising a nucleotide sequence, inserted into the chimeric site, wherein the heterologous nucleic acid chimeric insertion sequence comprises a pseudo random selection of nucleotides, capable of encoding a selection of amino acids;
b. Isolating viable chimeric virus expressing the chimeric region; and
c. Selecting virus, wherein the chimeric rhinovirus is capable of participating in an immune reaction.

35. An isolated monoclonal antibody which recognizes the chimeric rhinovirus of claim 1.

36. The isolated monoclonal antibody of claim 35, wherein the epitope that binds or is recognized by the antibody is within at least a portion of SEQ IS NO: 1.

37. The isolated monoclonal antibody of claim 35, wherein the epitope that binds or is recognized by the antibody is within at least a portion of SEQ IS NO: 2.

38. The antibody according to claim 35, wherein the antibody binds HIV.

39. An antibody which competes with the antibody of claim 35 for binding to HIV.

40. A monoclonal antibody producing cell line that produces a monoclonal antibody according to claim 35.

41. The method of claim 19, further comprising a step of boosting immunization with at least one peptide encoded by a nucleic acid having the ability to hybridize under standard hybridization conditions to a nucleic acid sequence or complement thereof, capable of encoding the sequence shown in SEQ ID NO.: 1.

42. The method of claim 41, wherein the peptide is KLH-conjugated.

43. The method of claim 19, further comprising a step of boosting immunization with at least one peptide encoded by a nucleic acid having the ability to hybridize under standard hybridization conditions to a nucleic acid sequence or complement thereof, capable of encoding the sequence shown in SEQ ID NO.: 2.

44. The method of claim 43, wherein the peptide is KLH-conjugated.
